(19) United States
(12) Patent Application Publication
(10) Pub. No.: US 2018/0153124 A1
(43) Pub. Date: Jun. 7, 2018

(54) FUSARIUM HEAD BLIGHT RESISTANCE IN PLANTS

(71) Applicant: Board of Trustees of Michigan State University, East Lansing, MI (US)

(72) Inventors: Masomeh Sticklen, East Lansing, MI (US); Hussien Alameldin, East Lansing, MI (US)

(21) Appl. No.: 15/827,390

(22) Filed: Nov. 30, 2017

Related U.S. Application Data
(60) Provisional application No. 62/428,841, filed on Dec. 1, 2016.

(51) Int. Cl. A01H 5/10; C12N 15/82; A01G 1/00; A01H 1/02

(52) U.S. Cl. CPC A01H 5/10; C12N 15/8282; A01H 1/02; C12N 15/8274; A01G 1/001; C12N 15/821

(57) ABSTRACT

Plants, plant cells, and plant seeds are described herein that are resistant to Fusarium head blight (FHB). The plants, plant cells, and plant seeds can be wheat plants, plant cells, and plant seeds.

This application claims benefit of priority to the filing date of U.S. Provisional Application Ser. No. 62/428,841, filed Dec. 1, 2016, the contents of which are specifically incorporated herein by reference in their entity.

### BACKGROUND OF THE INVENTION

The wheat (Triticum aestivum) Fusarium head blight (FHB), is mostly caused by Fusarium graminearum and has resulted in losses of $3 billion/year in North America. This pathogen not only reduces the crop yield, but also contains deoxynivalenol (DON), a mycotoxin that is harmful to the human and animal health. Several reports confirm the resistance of Chinese wheat lines to FHB: Suami 3 and Wangshuibai. However, strategies to transfer the FHB resistance genes into economically important wheat via conventional breeding have not been successful, due in part because resistance to the FHB pathogen is a complex trait and breeding of these two genotypes with agronomically important wheat lines is very difficult as. It has been reported that a few genes including the Thaumatin-Like Protein1 (tlp1) and the gene coding for the involvement of the coronatine insensitive 1-like protein (coi1) receptor are important in response to infection by the FHB pathogen.

### SUMMARY

Described herein are expression systems that provide resistance to Fusarium head blight (FHB). Wheat plants that include such expression systems are at least 2-fold to 5-fold more resistant to FHB than wild type or parent plant lines that do not have the expression systems.

For example, the expression systems described herein can have an expression cassette comprising at least one promoter operably linked to a nucleic acid that encodes a COI1 protein, a Tlp1 protein, or a combination thereof. In some cases, the expression system can hare two expression cassettes, a first expression cassette comprising a first promoter operably linked to a nucleic acid that encodes a COI1 protein, and a second promoter operably linked to a nucleic acid that encodes a Tlp1 protein. The expression systems provide enhanced levels of COI1 and/or Tlp1 proteins, which provides plants with improved resistance to Fusarium head blight (FHB).

Also described herein are plants, plant cells, and plant seeds that have the expression systems, as well as methods of making FHB-resistant plant cells, plants, and plant seeds.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1 illustrates structures of expression cassette constructs pjBarTlp and pjBarCoi.

FIG. 2 graphically illustrates levels of expression of tlp1 and coi1 genes as detected by quantitative polymerase chain reaction (qPRC) of mRNA obtained from in six independently transformed wheat lines.

FIG. 3 illustrates symptoms of the Fusarium head blight (FHB) pathogen Fusarium graminearum cell-free mycotoxin after single spot microinjection into wheat (site of injection is shown as single black spot on each spike) where the head of a wild type control plant is shown on the left and the head of a first generation (T0) plant that over-expresses TLP and COI1 is shown on the right 21 days after inculcation. Note that the T0 plants spikes that over-express TLP and COI1 are expected to be smaller than their control plant spikes, but T1 plants will have the normal size spikes.

FIG. 4 graphically illustrates the area under the disease progress curve (AUDPC) rates for the non-transgenic (wild type) plants versus the six different independent transgenic lines that over-express TLP and COI1.

FIG. 5 graphically illustrates the mass of 100 seeds from wild type wheat plants (rightmost bar) compared to the mass of 100 seeds from transgenic plants that overexpress COI1 and TLP (leftmost bar), the mass of 100 seeds from transgenic plants that overexpress COI1 (second from the left bar), and the mass of 100 seeds from transgenic plants that overexpress TLP (third from the left bar).

FIG. 6 illustrates the estimated mass per plant of transgenic plants that overexpress COI1 and TLP (leftmost bar), transgenic plants that overexpress COI1 (second from the left bar), and transgenic plants that overexpress TLP (third from the left bar), compared to the mass per plant of wild type plants.

FIG. 7 graphically illustrates the seed numbers per head of transgenic plants that overexpress COI1 and TLP (leftmost bar), transgenic plants that overexpress CO (second from the left bar), and transgenic plants that overexpress TLP (third from the left bar), compared to the seed numbers per head per plant of wild type plants.

### DETAILED DESCRIPTION

Described herein are expression cassettes, plant cells, plants, and plant seeds that include heterologous nucleic acids that encode polypeptides that confer resistance to Fusarium head blight (FHB). As shown herein, enhanced expression of CORONATINE INSENSITIVE 1 (COI1) protein and Thaumatin-Like Protein (tlp1) reduces the rate of FHB disease by at least two-fold to five-fold.

### Coronatine Insensitive 1 (COI1)

COI1 is an F-box protein that can mediate jasmonate signaling by promoting hormone-dependent ubiquitylation and degradation of transcriptional repressor JASMONATE ZIM DOMAIN (JAZ) proteins. JAZ proteins are repressors of the jasmonic acid signaling pathway. COI1 proteins can form a co-receptor with one or more JAZ transcriptional repressor protein that can bind jasmonate. Formation, or lack of formation, of jasmonate/COI1/JAZ complexes can regulate the sophisticated, multilayered immune signaling network present in plants.

The stress hormone jasmonate (JA) plays a central role in regulating plant defenses against a variety of chewing insects and necrotrophic pathogens. Salicylic acid (SA) is another plant hormone that can be employed for plant defense against biotrophic or hemibiotrophic pathogens. During host-pathogen coevolution, however, many successful plant pathogens developed mechanisms to attack or hijack components of the plant immune signaling network as part of their pathogenesis strategies. As a result, the plant immune system, although powerful, is often fallible in the face of highly evolved pathogens.

The COI1 protein expressed by the expression cassette, plant cells, plants, and plant seeds can have a variety of sequences. An example of a COI1 protein from Triticum aestivum (wheat) with NCBI accession number ADK66973.1 has the following sequence (SE ID NO:1).

| | |
|---|---|
| 1 | MGGEAPEPRR LSRALSLDGG GVPEEALHLV LGYVDDPRDR |
| 41 | EAASLACRRW HHIDALTRKH VTVPFCYAVS PARLLARFPR |
| 61 | LESLGVKGKP RAAMYGLIPD DWGAYARPWV AELAAPLECL |
| 121 | KALHLRRMVV TDDDLAALVR ARGHMLQELK LDKCSGFSTD |
| 161 | ALRLVARSCR SLRTLFLEEC TITDNGTEWL HDLAANNPVL |
| 201 | VTLNFYLTYL RVEPADLELL AKNCKSLISL KISDCDLSDL |
| 241 | IGFFQIATSL QEFAGAEISE QKYGNVKLPS KLCSFGLTFM |
| 281 | GTNEMHIIFP FSAVLKKLDL QYSFLTTEDH CQLIAKCPNL |
| 321 | LVLAVRNVIG DRGLGVVGDT CKKLQRLRVE RGEDDPGMQE |
| 361 | EEGGVSQVGL TAIAVGCREL ENIAAYVSDI TNGALESIGT |
| 401 | FCKNLHDFRL VLLDKQETIT DLPLDNGARA LLRGCTKLRR |
| 441 | FALYLRPGGL SDVGLGYIGQ HSGTIQYMLL GNVGQTDGGL |
| 481 | ISFAAGCRNL RKLELRSCCF SERALALAIR QMPSLRYVWV |
| 521 | QGYRASQTGR DLMLMARPFW NIEFTRPSTE TAGRLMEDGE |
| 561 | PCVDRQAQVL AYYSLSGKRS DYPQSVVPLY PA |

An example of a nucleotide (cDNA) sequence that encodes the SEQ ID NO:1 COI1 protein (NCBI accession number HM447645.1) is shown below as SEQ ID NO:2.

| | |
|---|---|
| 1 | ACGAGCACCA CCATCGGAGA AGGGCCAGCG GGAAGGGGGG |
| 41 | AAATCAATCC CCATGCCCCC ACCCCTCGCC GGACCAGATC |
| 81 | CCCGGCGGGC CGGCGCGGAG CCTTAGGCGG GGATGGGCGG |
| 121 | GGAGGCCCCG GAGCCGCGGC GGCTGAGCCG CGCGCTCAGC |
| 161 | CTGGACGGCG GCGGCGTCCC GGAGGAGGCG CTGCACCTGG |
| 201 | TGCTCGGCTA CGTGGACGAC CCGCGCGACC GCGAGGCGGC |
| 241 | CTCGCTGGCG TGCCGCCGCT GGCACCACAT CGACGCGCTC |
| 281 | ACGCGGAAGC ACGTCACCGT GCCCTTCTGC TACGCCGTGT |
| 321 | CCCCGGCGCG CCTGCTCGCG CGCTTCCCGC GCCTCGAGTC |
| 361 | GCTCGGGGTC AAGGGCAAGC CCCGCGCCGC CATGTACGGC |
| 401 | CTCATCCCCG ACGACTGGGG CGCCTACGCC CGGCCCTGGG |
| 441 | TCGCCGAGCT CGCCGCCCCG CTCGAGTGCC TCAAGGCGCT |
| 481 | CCACCTGCGC CGCATGGTCG TCACCGACGA CGACCTCGCC |
| 521 | GCCCTCGTCC GCGCCCGCGG CCACATGCTG CAGGAGCTCA |
| 561 | AGCTCGACAA GTGCTCCGGC TTCTCCACCG ACGCCCTCCG |
| 601 | CCTCGTCGCC CGCTCCTGCA GATCACTGAG AACTTTGTTT |
| 641 | CTGGAAGAAT GTACAATTAC TGATAATGGC ACTGAATGGC |
| 681 | TCCATGACCT TGCTGCCAAC AATCCTGTTC TGGTGACCTT |
| 721 | GAACTTCTAC TTGACTTACC TCAGAGTGGA GCCAGCTGAC |
| 761 | CTCGAGCTTC TCGCCAAGAA TTGCAAGTCA CTAATTTCGT |
| 801 | TGAAGATTAG CGACTGCGAC CTTTCAGATT TGATTGGATT |
| 841 | TTTCCAAATA GCTACATCTT TGCAAGAATT TGCTGGAGCG |
| 881 | GAAATCAGTG AGCAAAAGTA TGGAAATGTT AAGCTTCCTT |
| 921 | CAAAGCTTTG CTCCTTCGGA CTTACCTTCA TGGGGACAAA |
| 961 | TGAGATGCAC ATAATCTTTC CTTTTTCTGC TGTACTCAAG |
| 1001 | AAGCTGGATT TGCAGTACAG TTTTCTCACC ACTGAAGATC |
| 1041 | ATTGCCAGCT CATTGCAAAA TGTCCAAACT TACTAGTCCT |
| 1081 | TGCGGTGAGG AATGTGATTG GGGATAGAGG ACTGGGGGTT |
| 1121 | GTCGGAGACA CATGCAAGAA GCTACAAAGG CTCAGAGTTG |
| 1161 | AGCGAGGGGA AGATGACCCT GGCATGCAAG AAGAGGAAGG |
| 1201 | CGGAGTTTCT CAAGTAGGCC TAACAGCCAT AGCCGTAGGT |
| 1241 | TGCCGTGAAC TGGAAAACAT AGCTGCCTAT GTGTCTGATA |
| 1281 | TCACAAATGG GGCCCTGGAA TCCATCGGAA CGTTCTGCAA |
| 1321 | AAATCTCCAT GACTTTCGCC TTGTCCTGCT TGACAAACAA |
| 1361 | GAGACGATAA CAGATTTGCC GCTGGACAAC GGTGCCCGCG |
| 1401 | CGCTGCTCAG GGGCTGCACC AAGCTTCGGA GGTTCGCTCT |
| 1441 | ATACCTGAGA CCAGGGGGGC TTTCAGATGT AGGCCTCGGC |
| 1481 | TACATCGGGC AGCACAGTGG AACCATCCAG TACA7GCTTC |
| 1521 | TGGGTAACGT CGGGCAGACG GATGGTGGAT TGATCAGTTT |
| 1561 | CGCAGCCGGG TGCCGGAACC TGCGGAAGCT TGAACTGAGG |
| 1601 | AGCTGTTGCT TCAGCGAGCG GGCTCTGGCC CTCGCCATAC |
| 1641 | GGCAAATGCC TTCCCTGAGG TATGTGTGGG TGCAGGGCTA |
| 1681 | CAGGGCCTCT CAGACCGGCC GCGACCTCAT GCTCATGGCG |
| 1721 | CGGCCCTTCT GGAACATCGA GTTTACGCCT CCCAGCACGG |
| 1761 | AGACCGCGGG CCGGCTGATG GAAGATGGGG AGCCCTGCGT |
| 1801 | TGATAGGCAA GCTCAGGTGC TGGCGTACTA CTCCCTTTCT |
| 1841 | GGGAAGAGGT CCGACTACCC GCAGTCTGTT GTTCCTCTGT |
| 1881 | ATCCTGCGTG ACTGTAAATA CATTAAGCCG GTATGGTGTC |
| 1921 | TCTCTGGGAC GGCCCCTGGC TGGCCCTCTG CGCTTCTCGG |
| 1961 | GCAATAAGGA TGTTTGTATG TGGGTATTGT ATGGATCTGG |
| 2001 | TAGATTTTCT AGCTGCTGTG TACTGGAATA AGCGCATTGG |
| 2041 | TATTTTTGCC TGGTACTCCT ATCTAATCTT AGGAAGATGT |
| 2081 | ATACTAAAGT AACATTGTGC GAGTGAACTG TGACACTATT |
| 2121 | GCGCTTGCTT CGCAGGCATA AGCTTGTCTG GTTTCCGCGG |
| 2161 | CCTGCCC |

Another example of a COI1 protein from Triticum aestivum (wheat) has NCBI accession number ADK66974.1 (GI:301318118), with the following sequence (SEQ ID NO:3).

| | |
|---|---|
| 1 | MGGEVPEPRR LSRALSFGVP DEALHLVMGY VDAPRDREAA |
| 41 | SLVCRRWHRI DALTRKHVTV AFCYAADPSR LLARFPRLES |
| 81 | LALKGRPRAA MYGLISDDWG AYAAPWVARL AAPLECLKAL |
| 121 | HLRRMTVTDD DVATLIRSRG HMLQELKLDK CSGFSTDALR |
| 161 | LVARSCRSLR TLFLEECVIT DEGGEWLHEL AVNNSVLVTL |
| 201 | NFYMTELKVV PADLELLAKN CKSLLSLKIS ECDLSDLIGF |
| 241 | FEAANALQDF AGGSFNEVGE LTKYEKVKFP PRVCFLGLTF |
| 281 | MGKNEMPVIF PFSASLKKLD LQYTFLTTED HCQLISKCPN |
| 321 | LFVLEVRNVI GDRGLEVVGD TCKKLRRLRI ERGDDDPGLQ |
| 361 | EEQGGVSQLG LTAVAVGCRD LEYIAAYVSD ITNGALESIG |
| 401 | TFCKNLYDFR LVLLDRQKQV TDLPLDNGVR ALLRSCTKLR |
| 441 | RFALYLRPGG LSDIGLDYIG QYSGNIQYML LGNVGESDHG |
| 481 | LIRFAIGCTN LRKLELRSCC FSEQALSLAV LHMPSLRYIW |
| 521 | VQGYKASPAG LELLLMARRF WNIEFTPRSP EGLFRMTLEG |
| 561 | EPCVDKQAQV LAYYSLAGQR QDCPDWVTPL HPAA |

An example of a nucleotide (cDNA) sequence that encodes the second Triticum aestivum (wheat) SEQ ID NO:3 wheat COI1 protein (NCBI accession number HM447646.1) is shown below as SEQ ID NO:4.

| | |
|---|---|
| 1 | ACGAGGCCCG CAAAGCCCAC CCCCGTAGCA GAAAGGGAGG |
| 41 | GAGGGAGGAG GAATCTCCGT CTCCACCTCC ACCTCCATGC |
| 81 | CCCCGCCCCC CGCCGGGCCC GGCCCAGATC TCCCGCGCGG |
| 121 | CGGCCGCTAG CCGATCCGAT CCGGCCCGAT GGGCGGGGAG |
| 161 | GTGCCGGAGC CGCGGCGGCT CAGCCGCGCG CTCAGCTTCG |
| 201 | GCGTGCCCGA CGAGGCGCTG CACCTCGTCA TGGGCTACGT |
| 241 | CGACGCCCCG CGCGACCGGG AGGCCGCCTC GCTCGTCTGC |
| 281 | CGCCGCTGGC ACCGCATCGA CGCGCTCACC CGCAAGCACG |
| 321 | TCACCGTCGC CTTCTGCTAC GCCGCCGACC CCTCGCGCCT |
| 361 | CCTCGCCCGC TTCCCGCGCC TCGAGTCGCT GGCCCTCAAG |
| 401 | GGCAGGCCGC GCGCCGCCAT GTACGGCCTC ATCTCCGACG |
| 441 | ACTGGGGCGC CTACGCCGCG CCCTGGGTCG CACGGCTCGC |
| 481 | CGCGCCGCTC GAGTGCCTAA AGGCGCTCCA CCTGCGACGC |
| 521 | ATGACCGTAA CCGACGACGA CGTCGCCACG CTCATCCGCT |
| 561 | CCCGCGGCCA CATGCTGCAG GAGCTCAAGC TCGACAAGTG |
| 601 | CTCCGGCTTC TCCACCGACG CGCTCCGCCT CGTCGCCCGC |
| 641 | TCCTGCAGAT CCTTAAGAAC ATTATTTCTT GAAGAATGCG |
| 681 | TGATTACTGA CGAAGGTGGT GAATGGCTTC ATGAACTTGC |
| 721 | TGTCAACAAT TCTGTTCTTG TGACACTGAA CTTCTACATG |
| 761 | ACTGAGCTCA AAGTGGTGCC GGCTGATCTG GAGCTTCTAG |
| 801 | CAAAGAACTG CAAATCATTA CTTTCTTTAA AGATCAGTGA |
| 841 | GTGTGACCTT TCAGACCTGA TTGGTTTTTT CGAAGCAGCC |
| 881 | AATGCATTGC AAGATTTTGC TGGAGGATCG TTCAATGAGG |
| 921 | TAGGAGAGCT AACAAAGTAT GAAAAAGTCA AGTTTCCACC |
| 961 | AAGAGTATGC TTCTTGGGGC TTACGTTCAT GGGGAAAAAT |
| 1001 | GAGATGCCTG TTATCTTCCC CTTTTCTGCT TCATTAAAGA |
| 1041 | AGCTGGACTT GCAGTACACT TTCCTCACCA CTGAGGATCA |
| 1081 | TTGCCAGCTT ATCTCAAAAT GCCCGAACCT ATTTGTTCTT |
| 1121 | GAGGTGAGGA ATGTGATAGG AGACAGAGGG CTGGAGGTTG |
| 1161 | TCGGCGATAC ATGCAAGAAG CTACGAAGAC TTCGAATTGA |
| 1201 | GCGAGGGGAT GATGATCCAG GTCTACAAGA AGAGCAAGGA |
| 1241 | GGAGTTTCTC AGTTAGGCCT GACAGCGGTA GCTGTTGGTT |
| 1281 | GCCGAGACCT GGAGTACATA GCTGCCTATG TATCTGATAT |
| 1321 | CACCAACGGT GCTCTCGAAT CCATCGGGAC CTTCTGCAAA |
| 1361 | AATCTCTACG ACTTCCGGCT TGTCCTGCTC GACAGACAAA |
| 1401 | AGCAGGTAAC TGATCTGCCA CTCGACAACG GTGTTCGTGC |
| 1441 | TCTGTTAAGG AGTTGCACCA AGCTCCGGAG ATTTGCTCTC |
| 1481 | TACCTGAGAC CTGGAGGGCT CTCAGACATA GGCCTCGACT |
| 1521 | ACATCGGGCA GTACAGCGGC AACATTCAGT ACATGCTGCT |
| 1561 | GGGCAACGTC GGTGAATCTG ACCACGGGTT GATCCGCTTT |
| 1601 | GCGATAGGAT GCACCAACCT GCGGAAGCTT GAGCTTCGGA |
| 1641 | GCTGCTGCTT CAGCGAGCAA GCCCTGTCCC TCGCGGTGCT |
| 1681 | CCACATGCCC TCGCTCAGGT ACATATGGGT GCAAGGCTAC |
| 1721 | AAAGCCTCTC CAGCAGGCCT CGAGCTCCTG CTCATGGCGA |
| 1761 | GGCGATTCTG GAACATCGAG TTCACGCCCC CCAGCCCCGA |
| 1801 | GGGCTTGTTC CGCATGACGC TTGAAGGAGA ACCCTGCGTG |
| 1841 | GATAAGCAGG CCCAGGTTCT TGCCTACTAC TCCCTTGCTG |
| 1881 | GGCAGAGGCA GGACTGCCCT GACTGGGTGA CCCCGTTGCA |
| 1921 | TCCAGCTGCA TGATTGATTG TAAATACAGT GTACTACATC |
| 1961 | AAGTTGTGTG TACGTAGGTA CTC7ACCTTA TTGCCCCTCG |
| 2001 | TCCCTTGGGC AACGATCGTG TCCGAATATG GTAGTAATTT |
| 2041 | GTATGGATGT AGATCATTAG CTAGCTGCTT TGGTGCCCTA |
| 2081 | ATAAGCTAGT GCTACTGTAG TGCTGTAGCT GAGGTGTAGT |
| 2121 | GCAATAAGTT GCTGTTGTCG CTTGTACTAC TATGTATGTA |
| 2161 | ATCCTGGGAA GTTGTATGCT AAAGTTGCTC CGTGCTCGT |

A comparison of the Triticum aestivum (wheat) COI1 SEQ ID NO:1 sequence and the Triticum aestivum (wheat) COI1 SEQ ID NO:3 sequence is shown below, illustrating that the two proteins have at least 79% sequence identity.

| 79.2% identity in 596 residues overlap; Score: 2468.0; Gap frequency: 1.2% | | |
|---|---|---|
| UserSeq1 | 1 | MGGEAPEPRRLSRALSLDGGGVPEEALHLVLGYVDDPRDREAASLACRRWHHIDALTRKH |
| UserSeq3 | 1 | MGGEVPEPRRLSRALSF---GVPDEALHLVMGYVDAPRDREAASLVCRRWHRIDALTRKH |
| | | **** *********** *** ****** **** ********* ***** ******** |
| UserSeq1 | 61 | VTVPFCYAVSPARLLARFPRLESLGVKGKPRAAMYGLIPDDWGAYARPWVAELAAPLECL |
| UserSeq3 | 58 | VTVAFCYAADPSRLLARFPRLESLALKGRPRAAMYGLISDDWGAYAAPWVARLAAPLECL |
| | | *** **** * ************ ** ********* ******* **** ******** |
| UserSeq1 | 121 | KALHLRRMVVTDDDLAALVRARGHMLQELKLDKCSGFSTDALRLVARSGRSLRTLFLEEC |
| UserSeq3 | 118 | KALHLRRMTVTDDDVATLIRSRGHMLQELKLDKCSGFSTDALRLVARSGRSLRTLFLEEC |
| | | ******** ***** * * * *************************************** |
| UserSeq1 | 181 | TITDNGTEWLHDLAANNPVLVTLNFYLTYLRVEPADLELLAKNCKSLISLKISDGDLSDL |
| UserSeq3 | 178 | VITDEGGEWLHELAVNNSVLVTLNFYMTELKVVPADLELLAKNCKSLLSLKISEGDLSDL |
| | | *** * **** ** ** ******** * * * ************** ***** ****** |
| UserSeq1 | 241 | IGFFQIATSLQEFAGAEISE----QKYGNVKLPSKLCSFGLTFMGTNEMHIIFPFSAVLK |
| UserSeq3 | 238 | IGFFEAANALQDFAGGSFNEVGELTKYEKVKFPPRVCFLGLTFMGKNEMPVIFPFSASLK |
| | | **** * ** *** * ** ** * * ****** *** ****** ** |
| UserSeq1 | 297 | KLDLQYSFLTTEDHCQLIAKCPNLLVLAVRNVIGDRGLGVVGDTCKKLQRLRVERGEDDP |
| UserSeq3 | 298 | KLDLQYTFLTTEDHCQLISKCPNLFVLEVRNVIGDRGLEVVGDTCKKLRRLRIERGDDDP |
| | | ****** *********** ***** ** ********** ********* *** *** *** |
| UserSeq1 | 357 | GMQEEEGGVSQVGLTAIAVGCRELENIAAYVSDITNGALESIGTFCKNLHDFRLVLLDKQ |
| UserSeq3 | 358 | GLQEEQGGVSQLGLTAVAVGCRDLEYIAAYVSDITNGALESIGTFCKNLYDFRLVLLDRQ |
| | | * *** ***** **** ***** ** *********************** ******** * |
| UserSeq1 | 417 | ETITDLPLDNGARALLRGCTKLRRFALYLRPGGLSDVGLGYTGQHSGTIQYMLLGNVGQT |
| UserSeq3 | 418 | KQVTDLPLDNGVRALLRSCTKLRRFALYLRPGGLSDIGLDYTGQYSGNIQYMLLGNVGES |
| | | ******** ***** ****************** ** **** ** ********** |
| UserSeq1 | 471 | DGGLISFAAGCRNLRKLELRSCCFSERALALAIRQMPSLRYVWVQGYRASQTGRDLMLMA |
| UserSeq3 | 478 | DHGLIRFAIGCTNLRKLELRSCCFSEQALSLAVLHMPSLRYIWVQGYKASPAGLELLLMA |
| | | * *** ** ** ************** ** ** ****** ***** ** * * *** |
| UserSeq1 | 537 | RPFWNIEFTPPSTETAGRLMEDGEPCVDRQAQVLAYYSLSGKRSDYPQSVVPLYPA |
| UserSeq3 | 538 | RRFWNIEFTPPSPEGLFRMTLEGEPCVDKQAQVLAYYSLAGQRQDCPDWVTPLHPA |
| | | * ********** * * ****** ********** * * * * * ** ** |

In another example, an Oryza sativa Indica Group COI1 protein with a sequence provided by the NCBI database as accession number EAY98249.1, is shown below as SEQ ID NO:5.

| | |
|---|---|
| 1 | MSFGGAGSIP EEALHLVLGY VDDPRDREAV SLVCRRWHRI |
| 41 | DALTRKHVTV PFCYAASPAH LLARFPRLES LAVKGKPRAA |
| 81 | MYGLIPEDWG AYARPWVAEL AAPLECLKAL HLRRMVVTDD |
| 121 | DLAALVRARG HMLQELKLDK CSGFSTDALR LVALSCRSLR |
| 161 | TLFLEECSIA DNGTEWLHDL AVNNPVLETL NFHMTELIVV |
| 201 | PADLELLAKK CKSLISLKIS DCDFSDLIGF FRMAASLQEF |
| 241 | AGGAFIEQGE LTKYGNVKFP SRLCSLGLTY MGTNEMPIIF |
| 281 | PFSALLKKLD LQYTELTTED HCQLIAKCPN LLVLAVRNVI |
| 321 | GDRGLGVVAD TCKKLQRLRV ERGDDDPGLQ EEQGGVSQVG |
| 361 | LTTVAVGCRE LEYIAAYVSD ITNGALESIG TECKNLCDFR |
| 401 | LVLLDREERI TDLPLDNGVR ALLRGCMKLR RFALYLRPGG |
| 441 | LSDTGLGYIG QYSGIIQYML LGNVGETDDG LIRFALGCEN |
| 481 | LRKLELRSCC FSEQALACAI RSMPSLRYVW VQGYKASKTG |
| 521 | HDLMLMARPF WNIEFTPPSS ENANRMREDG EPCVDSQAQI |
| 561 | LAYYSLAGKR SDCPRSVVPL YPA |

A comparison of the Triticum aestivum (wheat) COI1 SEQ ID NO:1 sequence and the Oryza sativa Indica Group COI1 protein COI1 SEQ ID NO:5 sequence is shown below, illustrating that the two proteins have at least 86% sequence identity.

| 86.8% identity in 577 residues overlap; Score: 2614.0; Gap frequency: 0.7% | | |
|---|---|---|
| UserSeq1 | 20 | GGVPEEALHLVLGYVDDPRDREAASLACRRWHHIDALTRKHVTVPFCYAVSPARLLARFP |
| UserSeq5 | 7 | GSIPEEALHLVLGYVDDPRDREAVSLVCRRWHRIDALTRKHVTVPFCYAASPAHLLARFP |
| | | * ******************** ** ***** **************** *** ****** |
| UserSeq1 | 80 | RLESLGVKGKPRAAMYGLIPDDWGAYARPWVAELAAPLECLKALHLRRMVVTDDDLAALV |
| UserSeq5 | 67 | RLESLAVKGKPRAAMYGLIPEDWGAYARPWVAELAAPLECLKALHLRRMVVTDDDLAALV |
| | | ***** ************** *************************************** |
| UserSeq1 | 140 | RARGHMLQELKLDKCSGFSTDAIRLVARSCRSLRTLFLEECTITDNGTEWLHDLAANNPV |
| UserSeq5 | 127 | RARGHMLQELKLDKCSGFSTDAIRLVALSCRSLRTLFLEECSIADNGTEWLHDLAVNNPV |
| | | *************************** ************* * *********** **** |
| UserSeq1 | 200 | LVTLNFYLTYLRVEPADLELLAKNCKSLISLKISDCDLSDLIGFFQIATSLQEFAGAEIS |
| UserSeq5 | 187 | LETLNFHMTELTVVPADLELLAKKCKSLISLKISDCDFSDLIGFFRMAASLQEFAGGAFI |
| | | * **** * * * ********* ************* ******* * ******* |
| UserSeq1 | 260 | EQ----KYGNVKLPSKLCSTGLTFMGTNEMHIIFPFSAVLKKLDLQYSFLTTEDHCQLIA |
| UserSeq5 | 247 | EQGELTKYGNVKEPSRLCSLGLTYMGTNEMPIIFPFSALLKKLDLQYTFLTTEDHCQLIA |
| | | ** ****** ** *** *** ****** ******* ******** ************ |
| UserSeq1 | 316 | KCPNLLVLAVRNVIGDRGLGVVGDTCKKLQRLRVERGEDDPGMQEEEGGVSQVGLTAIAV |
| UserSeq5 | 307 | KCPNLLVLAVRNVIGDRGLGVVADTCKKLQRLRVERGDDDPGLQEEQGGVSQVGLTTVAV |
| | | ********************** ************** **** *** ********* ** |
| UserSeq1 | 376 | GCRELENIAAYVSDITNGALESIGTFCKNLHDFRLVLLDKQETITDLPLDNGARALLRGC |
| UserSeq5 | 367 | GCRELEYIAAYVSDITNGALESIGTFCKNLCDFRLVLLDREERITDLPLDNGVRALLRGC |
| | | ****** *********************** ******** * ********* ******* |
| UserSeq1 | 436 | TKLRRFALYLRPGGLSDVGLGYIGQHSGTIQYMLLGNVGQTDGGLISFAAGCRNLRKLEL |
| UserSeq5 | 427 | MKLRRFALYLRPGGLSDTGLGYIGQYSGIIQYMLLGNVGETDDGLIRFALGCENLRKLEL |
| | | **************** ******* ** ********** ** *** ** ** ******* |
| UserSeq1 | 496 | RSCCFSERALALAIRQMPSLRYVWVQGYRASQTGRDLMLMARPFWNIEFTPPSTETAGRL |
| UserSeq5 | 481 | RSCCFSEQALACAIRSMPSLRYVWVQGYKASKTGHDLMLMARPFWNIEFTPPSSENANRM |
| | | ******* *** *** ************ ** ** ****************** * * * |
| UserSeq1 | 336 | MEDGEPCVDRQAQVLAYYSLSGKRSDYPQSVVPLYPA |
| UserSeq5 | 347 | REDGEPCVDSQAQILAYYSLAGKRSDCPRSVVPLYPA |
| | | ******** *** ****** ***** * ******** |

In another example, a Hordeum vulgare subsp. vulgare (domesticated barley) COI1 protein with a sequence provided by the NCBI database as accession number BAJ94334.1, is shown below as SEQ ID NO:6.

| | |
|---|---|
| 1 | MGGEAPEPRR LTRALSVDGS GVPEEALHLV FGYVDDPRDR |
| 41 | EAASLACRRW HHIDALTRKH VTVPFCYAVS PARLLARFPR |
| 81 | LESLGVKGKP RAAMYGLISD DWGAYARPWI AELAAPLECL |
| 121 | KALHLRRMVV TDDDLAALVL ARGHMLQELK LDKCSGESTD |
| 161 | ALRLVARSCR SLRILFLEEC TITDNGTEWL HDLAANNPVL |
| 201 | VNLNFYLTYL RAVPADLELL ARNCKSLISL KISDCDLSDL |
| 241 | VGFFQIATSL QEFAGAEISE QMYGNVKFPS KICSFGLTFM |
| 281 | GINEMHIIFP FSAVLKKLDL QYSFLTTEDH CQLIAKCPNL |
| 321 | LVLAVRNVIG DRGLAVVGDT CKKLQRLRVE RGEDDPGMQE |
| 361 | EGGVSQVGLT AVAVGCRELE YIAAYVSDIT NGALESIGTF |
| 401 | CKKLYDFRLV LLDRQERITD LPLDNGARAL LRGCTKLRRF |
| 441 | ALYLRPGGLS DVGLNYIGQH SGTIHYMLLG NVGQTDDGLI |
| 481 | SFAAGCRNLL KLELRSCCFS ERALALAVLK MPSLRYVWVQ |
| 521 | GYRASQTGRD LMLMARPFWN IEFTPPGTES AGRLMEDGEP |
| 561 | CVDRQAQVLA YYSLSGRRSD CPQSVVPLYP A |

An example of a nucleotide (cDNA) sequence that encodes the SEQ ID NO:6 Hordeum vulgare subsp. vulgare COI1 protein (NCBI accession number AK363130.1) is shown below as SEQ ID NO:7.

| | |
|---|---|
| 1 | GGTGGCAAAA TCCCCATGCC TCAGCCCCTC GCCGGACCAG |
| 41 | ATCCCCGGCG AGCCAGCGCG GGGGATTAGG CGGGGAGAGG |
| 81 | CCCGATCGAT GGGCGGGGAG GCCCCGGAGC CGCGGCGGCT |
| 121 | GACCCGCGCG CTCAGCGTGG ACGGCAGCGG TGTCCCGGAG |
| 161 | GAGGCGCTGC ACCTGGTGTT CGGGTACGTC GACGACCCGC |
| 201 | GCGACCGGGA GGCGGCGTCG CTGGCCTGCC GCCGGTGGCA |
| 241 | CCACATCGAC GCGCTCACGC GGAAGCATGT CACCGTGCCC |
| 281 | TTCTGCTACG CGGTTTCCCC GGCACGCCTG CTCGCGCGCT |
| 321 | TCCCGCGCCT CGAGTCGCTC GGGGTCAAGG GCAAGCCCCG |
| 361 | CGCCGCCATG TACGGCCTCA TCTCCGACGA CTGGGGCGCC |
| 401 | TACGCTCGCC CCTGGATAGC CGAGCTCGCT GCCCCGCTCG |
| 441 | AGTGCCTCAA GGCGCTCCAC CTGCGCCGCA TGGTCGTCAC |
| 481 | CGACGACGAC CTCGCCGCCC TTGTCCTCGC CCGCGGCCAC |
| 521 | ATGCTGCAGG AGCTCAAGCT CGACAAGTGC TCTGGCTTCT |
| 561 | CCACCGACGC CCTCCGCCTC GTCGCCCGAT CCTGCAGATC |
| 601 | ACTGAGAACT TTGTTTCTGG AAGAATGCAC AATTACTGAT |
| 641 | AATGGCACTG AATGGCTCCA TGATCTTGCT GCCAACAATC |
| 681 | CTGTTCTGGT GAACTTGAAC TTCTACTTGA CTTACCTCAG |
| 721 | AGCGGTGCCA GCTGACCTCG AGCTTCTTGC CAGGAATTGC |
| 761 | AAGTCACTAA TTTCATTGAA GATCAGTGAT TGTGACCTTT |
| 801 | CAGATTTAGT TGGATTTTTC CAAATAGCTA CGTCATTGCA |
| 841 | AGAATTTGCT GGAGCGGAAA TTAGTGAGCA AATGTATGGA |
| 881 | AATGTTAAGT TTCCTTCAAA GATTTGCTCA TTCGGACTTA |
| 921 | CCTTCATGGG GATAAATGAG ATGCACATAA TCTTTCCTTT |
| 961 | TTCCGCTGTA CTCAAGAAGC TGGATTTGCA GTACAGTTTC |
| 1001 | CTCACCACTG AAGATCATTG CCAGCTCATT GCAAAATGTC |
| 1041 | CAAACTTACT AGTTCTTGCG GTGAGGAATG TGATTGGGGA |
| 1081 | TAGAGGATTA GCGGTTGTCG GAGACACATG CAAGAAGCTA |
| 1121 | CAAAGGCTCA GAGTTGAGAG AGGGGAAGAT GATCCTGGTA |
| 1161 | TGCAAGAAGA AGGAGGAGTT TCTCAAGTAG GCCTAACAGC |
| 1201 | CGTAGCCGTA GGCTGCCGTG AACTGGAATA CATAGCCGCC |
| 1241 | TATGTGTCTG ATATCACGAA CGGGGCCCTA GAATCTATCG |
| 1281 | GAACATTCTG CAAAAAGCTT TATGACTTTC GCCTTGTCCT |
| 1321 | GCTTGACAGA CAAGAGAGGA TAACAGATTT GCCACTGGAC |
| 1361 | AATGGTGCCC GTGCGCTGCT GAGGGGCTGC ACTAAACTTC |
| 1401 | GGAGGTTCGC TCTATACCTG AGACCAGGGG GCCTTTCAGA |
| 1441 | TGTGGGCCTT AACTATATTG GACAGCACAG TGGAACTATC |
| 1481 | CACTACATGC TTCTGGGTAA CGTTGGGCAA ACGGATGACG |
| 1521 | GATTAATCAG TTTTGCAGCT GGGTGCCGGA ACCTGCTGAA |
| 1561 | GCTTGAATTA AGGAGCTGCT GCTTCAGCGA GCGGGCTTTG |
| 1601 | GCCCTCGCCG TACTGAAAAT GCCTTCTCTG AGGTACGTAT |
| 1641 | GGGTGCAGGG CTACAGAGCC TCTCAAACTG GCCGCGACCT |
| 1681 | CATGCTCATG GCAAGGCCCT TCTGGAACAT TGAGTTTACG |
| 1721 | CCTCCCGGCA CGGAGAGCGC GGGTCGGCTG ATGGAAGATG |
| 1761 | GGGAGCCCTG TGTTGATAGG CAAGCTCAGG TACTTGCATA |
| 1801 | CTACTCCCTT AGTGGGAGGA GGTCGGACTG CCCGCAGTCT |
| 1841 | GTTGTTCCTC TGTATCCTGC GTGACTGTAC ATACACAAAG |
| 1881 | CTGGCGCATG TTTGCGATGG TGTAGCCCCC GGGCCCTTCT |
| 1921 | TGGGCAATAA GGATATGTTT GTATGTGGGT ATTGTATGGA |
| 1961 | TCTAGTAGAT GTCTAGCTGC TGTGTACTGG AATAAGCGCA |
| 2001 | TGCTATTTTT GCCTGGTACT CCTATCTAAT CCTAGGAAGA |
| 2041 | TGTATACTAA AGTAACAATG TGTGGGTGCA ACTGTGACAC |
| 2081 | TATTGTGCTT GCTCCCAGGT ATAAGCATGC CCGGTTTTTG |
| 2121 | CAACATGTTC TCTGTTGTAC ATAATTGCTC TCTGAAAAAA |
| 2161 | AAAATCTCCT GGTG |

A comparison of the Triticum aestivum (wheat) COI11 SEQ ID NO:1 sequence and the Hordeum vulgare subsp. vulgare COI1 protein with SEQ ID NO:6 sequence is shown below, illustrating that the two proteins have at least 94% sequence identity.

| 94.1% identity in 592 residues overlap; Score: 2899.0; Gap frequency: 0.2% | | |
|---|---|---|
| UserSeq1 | 1 | MGGEAPEPRRLSRALSLDGGGVPEEALHLVLGYVDDPRDREAASLACRRWHHIDALTRKH |
| UserSeq6 | 1 | MGGEAPEPRRLTRALSVDGSGVPEEALHLVFGYVDDPRDREAASLACRRWHHIDALTRKH |
| | | *********** **** ** ********** ***************************** |
| UserSeq1 | 61 | VTVPFCYAVSPARLLARFPRLESLGVKGKPRAANYGLIPDDWGAYARDWVAELAAPLECL |
| UserSeq6 | 61 | VTVPFCYAVSPARLLARFPRLESLGVKGKPRAANYGLISDDWGAYARDWIAELAAPLECL |
| | | ************************************** ********** ********** |
| UserSeq1 | 121 | KALHLRRMVVTDDDLAALVRARGHMLQELKLDKCSGFSTDALRLVARSCRSLRTLFLEEC |
| UserSeq6 | 121 | KALHLRRMVVTDDDLAALVLARGHMLQELKLDKCSGFSTDALRLVARSCRSLRTLFLEEC |
| | | ******************* **************************************** |
| UserSeq1 | 181 | TITDNGTEWLHDLAANNPVLVTLNFYLTYLRVEPADLELLAKNCKSLISLKISDCDLSDL |
| UserSeq6 | 181 | TITDNGTEWLHDLAANNPVLVNLNFYLTYLRAVPADLELLARNCKSLISLKISDCDLSDL |
| | | ********************* ********* ******** ****************** |
| UserSeq1 | 241 | IGFFQTATSLQEFAGAEISEQKYGNVKLPSKLCSEGLTFMGTNEMHIIFPFSAVLKKLDL |
| UserSeq6 | 241 | VGFFQTATSLQEFAGAEISEQMYGNVKIPSKICSEGLTFMGINEMHIIFPFSAVLKKLDL |
| | | ******************** ***** *** ********* ******************* |
| UserSeq1 | 301 | QYSFLTTEDHCQLIAKCPNLLVLAVRNVIGDRGLGVVGDTCKKLQRLRVERGEDDPGMQE |
| UserSeq6 | 301 | QYSFLTTEDHCQLIAKCPNLLVLAVRNVIGDRGLAVVGDTCKKLQRLRVERGEDDPGMQE |
| | | ********************************** ************************* |
| UserSeq1 | 361 | EEGGVSQVGLTAIAVGCRELENIAAYVSDITNGALESIGTFCKNLHDFRLVLLDKQETIT |
| UserSeq6 | 361 | E-GGVSQVGLTAVAVGCRELEYIAAYVSDITNGALESIGTFCKKLYDFRLVLLDRQERIT |
| | | * ********** ******** ********************* * ******** ** ** |
| UserSeq1 | 421 | DLPLDNGARALLRGCTKLRRFALYLRPGGLSDVGLGYIGQHSGTIQYMLLGNVGQTDGGL |
| UserSeq6 | 420 | DLPLDNGARALLRGCTKLRRFALYLRPGGLSDVGLNYIGQHSGTIHYMLLGNVGQTDDGL |
| | | *********************************** ********* *********** ** |
| UserSeq1 | 481 | ISFAAGCRNLRKLELRSCCFSERALALAIRQMPSLRYVWVQGYRASQTGRDLMLMARPFW |
| UserSeq6 | 480 | ISFAAGCRNLLKLELRSCCFSERALALAVIKMPSLRYVWVQGYRASQTGRDLMLMARPFW |
| | | ********** ***************** ***************************** |
| UserSeq1 | 141 | NIEFTPPSTETAGRLMEDGEPCVDRQAQVLAYYSLSGKRSDYPQSVVPLYPA |
| UserSeq6 | 540 | NIEFTPPGTESAGRLMEDGEPCVDRQAQVLAYYSLSGRRSDCPQSVVPLYPA |
| | | ******* ** ************************** *** ********** |

In another example, a second Hordeum vulgare subsp. vulgare (domesticated barley) COI1 protein with a sequence provided by the NCBI database as accession number BAJ90363.1, is shown below as SEQ ID NO:8.

| | |
|---|---|
| 1 | MGGEAPEPRR LTRALSVDGS GVPEEALHLV FGYVDDPRDR |
| 41 | EAASLACRRW HHIDALTRKH VTVPFCYAVS PARLLARFPR |
| 81 | LESLGVKGKP RAAMYGLISD DWGAYARPWI AELAAPLECL |
| 121 | KALHLRRMVV TDDDLAALVL ARGHMLQELK LDKCSGFSTD |
| 161 | ALRLVARSCR SLRTLFLEEC TITDNGTEWL HDLAANNPVL |
| 201 | VNLNFYLTYL RAVPADLELL ARNCKSLISL KISDCDLSDL |
| 241 | VGFFQIATSL QEFAGAEISE QMYGNVKFPS KICSFGLTFM |
| 281 | GINEMHIIFP FSAVLKKLDL QYSFLTTEDH CQLIAKCPNL |
| 321 | LVLAVRNVIG DRGLAVVGDT CKKLQRLRVE RGEDDPGMQK |
| 361 | EGGVSQVGLT AVAVGCRELE YIAAYVSDIT NGALESIGTF |
| 401 | CKKLYDFRLV LLDRQERITD LPLDNGARAL LRGCTKLRRF |
| 441 | ALYLRPGGLS DVGLNYIGQH SGTIHYMLLG NVGQTDDGLI |
| 481 | SFAAGCRNLL KLELRSCCFS ERALALAVLK MPSLRYVWVQ |
| 521 | GYRASQTGRD LMLMARPFWN IEFTPPGTES AGRLMEDGEP |
| 561 | CVDRQAQVLA YYSLSGRRSD CPQSVVPLYP A |

An example of a nucleotide (cDNA) sequence that encodes the SEQ ID NO:8 Hordeum vulgare subsp. vulgare COI1 protein (NCBI accession number AK359152.1) is shown below as SEQ ID NO:9.

| | |
|---|---|
| 1 | GGAGGCAAAA TCCCCATGCC TCAGCCCCTC GGCGGACCAG |
| 41 | ATCCCCGGCG AGCCAGCGCG GGGGATTAGG CGGGGAGAGG |
| 81 | CCCGATCGAT GGGCGGGGAG GCCCCGGAGC CGCGGCGGCT |
| 121 | GACCCGCGCG CTCAGCGTGG ACGGCAGCGG CGTCCCGGAG |
| 161 | GAGGCGCTGC ACCTGGTGTT CGGGTACGTC GACGATCCGC |
| 201 | GCGACCGGGA GGCGGCGTCG CTGGCCTGCC GCCGGTGGCA |
| 241 | CCACATCGAC GCGTTCACGC GGAAGCATGT CACCGTGCCC |
| 281 | TTCTGCTACG CGGTTTCCCC GGCACGCCTG CTCGCGCGCT |
| 321 | TCCCGCGCCT CGAGTCGCTC GGGGTCAAGG GCAAGCCCCG |
| 361 | CGCCGCCATG TACGGCCTCA TCTCCGACGA CTGGGGCGCC |
| 401 | TACGTTCGTC CCTCGATAGT CGAGCTCGCT GCCCCGCTCG |
| 441 | AGTGCCTCAA GGCGCTCCAC CTGCGCCGCA TGGTCGTCAC |
| 481 | CGACGATGAC CTCGCCGCCC TTGTCCTCGC CCGCGGCCAT |
| 521 | ATGCTGTAGG AGCTCAAGCT CGACAAGTGC TCTGGTTTCT |
| 561 | CCACCGACGC CCTCCGCCTC GTCGCCCGAT CCTGCAGATC |
| 601 | ACTGAGAATT TTGTTTCTGG AAGAATGCAC AATTACTGAT |
| 641 | AATGGCACTG AATGGCTCCA TGATCTTGCT GCCAACAATC |
| 681 | CTGTTCTGGT GAACTTGAAC TTCTACTTGA CTTACCTCAG |
| 721 | AGCGGTGCCA GCTGACCTCG AGCTTCTTGC CAGGAATTGC |
| 761 | AAGTCATTAA TTTCATTGAA GATCAGTGAT TGTGATCTTT |
| 801 | CAGATTTAGT TGGATTTTTC CAAATAGCTA CGTCATTGCA |
| 841 | AGAATTTGCT GGAGCGGAAA TTAGTGAGTA AATGTATGGA |
| 881 | AATGTTAAGT TTCCTTCAAA GATTTGCTCA TTCGGACTTA |
| 921 | CCTTCATGGG GATAAATGAG ATGCACATAA TCTTTCCTTT |
| 961 | TTCCGCTGTA CTCAAGAAGC TGGATTTGCA GTATAGTTTC |
| 1001 | CTCACCACTG AAGATCATTG CCAGCTCATT GCAAAATGTC |
| 1041 | CAAACTTACT AGTTCTTGCG GTGAGGAATG TGATTGGGGA |
| 1081 | TAGAGGATTA GCGGTTGTCG GAGATACATG CAAGAAGCTA |
| 1121 | CAAAGGCTCA GAGTTGAGAG AGGGGAAGAT GATCCTGGTA |
| 1161 | TCCAAAAAGA AGGAGGAGTT TCTCAAGTAG GCCTAACAGC |
| 1201 | CGTAGCCGTA GGCTGCCGTG AATTGGAATA CATAGCCGCC |
| 1241 | TATGTGTCTG ATATCACGAA CGGGGCCCTA GAATCTATCG |
| 1281 | GAACATTCTG CAAAAAGCTT TATGACTTTC GCCTTGTCCT |
| 1321 | GCTTGACAGA CAAGAGAGGA TAACAGATTT GCCACTGGAC |
| 1361 | AATGGTGCCC GTGCGCTGCT GAGGGGCTGC ATTAAACTTC |
| 1401 | GGAGGTTCGC TCTATACCTG AGACCAGGGG GCCTTTCAGA |
| 1441 | TGTGGGTCTT AACTATATTG GACAGCACAG TGGAACTATC |
| 1481 | CACTACATGC TTCTGGGTAA CGTTGGGCAA ACGGATGACG |
| 1521 | GATTAATCAG TTTTGCAGCT GGGTGCCGGA ACCTGCTGAA |
| 1561 | GCTTGAATTA AGGAGCTGCT GCTTCAGCGA GCGGGCTTTG |
| 1601 | GCCCTCGCCG TACTGAAAAT GCCTTCTCTG AGGTACGTAT |
| 1641 | GGGTGCAGGG CTACAGAGCC TCTCAAACTG GCCGCGACCT |
| 1681 | CATGCTCATG GCAAGGCCCT TCTGGAACAT TGAGTTTACG |
| 1721 | CCTCCCGGCA CGGAGAGCGC GGGTCGGCTG ATGGAAGATG |
| 1761 | GGGAGCCCTG TGTTGATAGG CAAGTTCAGG TACTTGCATA |
| 1801 | CTACTCCCTT AGTGGGAGGA GGTCGGACTG CCCGCAGTCT |
| 1841 | GTTGTTCCTC TGTATCCTGC GTGACTGTAC ATATACAAAG |
| 1881 | CTGGTGCATG TTTGCGATGG TGTAGCCCCC GGGTCCTTCT |
| 1921 | TGGGCAATAA GGATATGTTT GTATGTGGGT ATTGTATGGA |
| 1961 | TCTAGTAGAT GTCTAGCTGC TGTGTACTGG AATAAGCGCA |
| 2001 | TGCTATTTTT GCCTCGT |

A comparison of the Triticum aestivum (wheat) COI1 SEQ ID NO:1 sequence and the second Hordeum vulgare subsp. vulgare COI1 protein with SEQ ID NO:8 sequence is shown below, illustrating that the two proteins have at least 93% sequence identity.

| 93.9% identity in 592 residues overlap; Score: 2895.0; Gap frequency: 0.2% | | |
|---|---|---|
| UserSeq1 | 1 | MGGEAPEPRRLSRALSLDGGGVPEEALHLVLGYVDDPRDREAASLACRRWHHIDALTRKH |
| UserSeq8 | 1 | MGGEAPEPRRLTRALSVDGSGVPEEALHLVFGYVDDPRDREAASLACRRWHHIDALTRKH |
| | | *********** **** ** ********** ***************************** |
| UserSeq1 | 61 | VTVPFCYAVSPARLLARFPRLESLGVKGKPRAAMYGLIPDDWGAYARPWVAELAAPLECL |
| UserSeq8 | 61 | VTVPFCYAVSPARLLARFPRLESLGVKGKPRAAMYGLISDDWGAYARPWIALLAAPLECL |
| | | ************************************** ********** ********** |
| UserSeq1 | 121 | KALHLPRMVVTDDDLAALVRARGHMLQELKLDKCSGFSTDALRLVARSCRSLRTLFLEEC |
| UserSeq8 | 121 | KALHLPRMVVTDDDLAALVLARGEMLQELKLDKCSGFSTDALRLVARSCRSLRTLFLEEC |
| | | ******************* **************************************** |
| UserSeq1 | 181 | TITDNGTEWLHDLAANNPVLVTLNFYLTYLRVEPADLELLAKNCKSLISLKISDCDLSDL |
| UserSeq8 | 181 | TITDNGTEWLHDLAANNPVLVNLNFYLTYLRAVPADLELLARNCKSLISLKISDCDLSDL |
| | | ********************* ********* ******** ****************** |
| UserSeq1 | 241 | IGFFQIATSLQEFAGAEISEQKYGNVKLPSKLCSFGLTFMGTNEMHIIFPFSAVLKKLDL |
| UserSeq8 | 241 | VGFFQIATSLQEFAGAEISEQMYGNVKFPSKICSFGLTFMGINEMHIIFPFSAVLKKLDL |
| | | ******************** ***** *** ********* ****************** |
| UserSeq1 | 301 | QYSFLTTEDHCQLIAKCPNLLVLAVRNVIGDRGLGVVGDTCKKLQRLRVERGEDDPGMQE |
| UserSeq8 | 301 | QYSFLTTEDHCQLIAKCPNLLVLAVRNVIGDRGLAVVGDTCKKLQRLRVERGEDDPGMQK |
| | | ********************************** ************************ |
| UserSeq1 | 361 | EEGGVSQVGLTAIAVGCRELENIAAYVSDITNGALESIGTFCKNLHDFRLVLLDKQETIT |
| UserSeq8 | 361 | E-GGVSQVGLTAVAVGCRELEYIAAYVSDITNGALESIGTFCKKLYDFRLVLLDRQERIT |
| | | * ********** ******** ********************* * ******** ** ** |
| UserSeq1 | 421 | DLPLDNGARALLRGCTKLRRFALYLRPGGLSDVGLGYIGQHSGTIQYMLLGNVGQTDGGL |
| UserSeq8 | 420 | DLPLDNGARALLRGCTKLRRFALYLRPGGLSDVGLNYIGQHSGTIHYMLLGNVGQTDDGL |
| | | *********************************** ********* *********** ** |
| UserSeq1 | 481 | ISFAAGCRNLRKLELRSCCFSERALALAIRQMPSLRYVWVQGYRASQTGRDLMLMARPFW |
| UserSeq8 | 480 | ISFAAGCRNLLKLELRSCCFSERALALAVLKMPSLRYVWVQGYRASQTGRDLMLMARPFW |
| | | ********** ***************** ***************************** |
| UserSeq1 | 541 | NIEFTPPSTETAGRLMEDGEPCVDRQAQVIAYYSLSGKRSDYPQSVVPLYPA |
| UserSeq8 | 540 | NIEFTPPGTESAGRLMEDGEPCVDRQAQVLAYYSLSGRRSDCPQSVVPLYPA |
| | | ******* ** ************************** *** ********** |

In another example, a Sorghum bicolor (sorghum) COI1 protein with a sequence provided by the NCBI database as accession number XP_002439888.1, is shown below as SEQ ID NO:10.

| | |
|---|---|
| 1 | MGGEAPEPRR LTRALSIGGG DGGWVPEEML HLVMGFVEDP |
| 41 | RDREAASLVC RRWHRVDALS RKHVTVPFCY AVSPARLLAR |
| 81 | FPRLESLAIK GKPRAAMYGL IPDDWGAYAR PWVAELAAPL |
| 121 | ECLKALHLRR MVVTDDDLAE LVRARGHMLQ ELKLDKCTGF |
| 161 | STDGLRLVAR SCRSLRTLFL EECQINDKGS EWIHDLADGC |
| 201 | PVLTTLNFHM TELQVMPADL EFLARSCKSL ISLKISDCDV |
| 241 | SDLIGFFQFA TALEEFAGGT FNEQGELTMY GNVRFPSRLC |
| 281 | SLGLTFMGTN EMPIIFPFSA ILKKLDLQYT VLTTEDHCQL |
| 321 | IAKCPNLLVL AVRNVIGDRG LGVVADTCKK LQRLRIERGD |
| 361 | DEGGVQEEQG GVSQVGLTAI AVGCRELEYI AAYVSDITNG |
| 401 | ALESIGTFCK KLYDFRLVLL DREERITELP LDNGVRALLR |
| 441 | GCTKLRRFAL YLRPGGLSDA GLGYIGQCSG NIQYMLLGNV |
| 481 | GETDDGLFSF ALGCVNLRKL ELRSCCFSER ALALAILRMP |
| 521 | SLRYVWVQGY KASQTGRDLM LMARPFWNIE FTPPSSENAG |
| 561 | RLMEDGEPCV DSHAQILAYH SLAGKRLDCP QSVVPLYPA |

An example of a nucleotide (cDNA) sequence that encodes the SEQ ID NO:10 Sorghum bicolor COI1 protein (NCBI accession number XM_002439843.1) is shown below as SEQ ID NO:11.

| | |
|---|---|
| 1 | CTCGTCCGTC CTCCTCTCCA CTCTCTCTTC TCCCTCCAAT |
| 41 | AATTCTCTCC TCTCTCTCTG CACTCTGCTT GCTCCACCTC |
| 81 | CAAGCACCAC CGAATCAGGG CCAGTGGGAG CAGCAGCAGC |
| 121 | AGCGAGTGGG AGCAGAGGAG GGCAGAGAAT CCCATGTCTC |
| 161 | CGCCCCTCGC TAGAGCAGAT CCTCGGCGAG CCGGGCGTGG |
| 201 | AGCTGCTTCG GTAGAAAAGC GAGCCAACTG AGCCTGCGAG |
| 241 | CGCCTGATCC GCCCGCGGCC CGATCGGGAT CGATGGGCGG |
| 281 | TGAGGCGCCG GAGCCCCGGC GGCTGACCCG CGCGCTGAGC |
| 321 | ATCGGCGGCG GCGACGGCGG CTGGGTCCCC GAGGAGATGC |
| 361 | TGCACCTGGT GATGGGGTTC GTCGAGGACC CGCGCGACCG |
| 401 | GGAGGCCGCG TCGCTGGTGT GCCGCCGGTG GCACCGCGTC |
| 441 | GACGCGCTGT CGCGGAAGCA CGTCACGGTG CCCTTCTGCT |
| 481 | ACGCCGTGTC CCCGGCGCGC CTGCTCGCGC GGTTCCCGCG |
| 521 | GCTCGAGTCG CTGGCCATCA AGGGGAAGCC CCGCGCGGCC |
| 561 | ATGTACGGCC TCATACCGGA CGACTGGGGC GCCTACGCCC |
| 601 | GCCCCTGGGT CGCCGAGCTC GCCGCGCCGC TCGAGTGCCT |
| 641 | CAAGGCGCTC CACCTCCGAT GCATGGTCGT CACGGACGAC |
| 681 | GACCTCGCCG AGCTCGTCCG TGCCAGGGGA CACATGCTGC |
| 721 | AGCAGCTCAA GCTCGACAAG TGTACCGGCT TCTCCACGGA |
| 761 | TGGACTCCGC CTCGTTGCGC GCTCCTGCAG ATCACTGAGA |
| 801 | ACTTTGTTTC TGGAAGAATG TCAAATTAAT GATAAAGGCA |
| 841 | GTGAATGGAT CCACGATCTT GCAGACGGTT GTCCTGTTCT |
| 881 | GATAACATTG AATTTCCACA TGACTGAGCT TCAAGTGATG |
| 921 | CCAGCTGACC TAGAGTTTCT TGCAAGGAGC TGCAAGTCAT |
| 961 | TGATTTCCTT GAAGATTAGC GACTGTGATG TTTCAGATTT |
| 1001 | GATAGGGTTC TTCCAATTTG CCACAGCACT GGAAGAATTT |
| 1041 | GCTGGAGGGA CATTCAATGA GCAAGGGGAA CTCACCATGT |
| 1081 | ATGGGAATGT CAGATTTCCA TCAAGATTAT GCTCCTTGGG |
| 1121 | ACTTACTTTC ATGGGAACAA ATGAAATGCC TATTATATTT |
| 1161 | CCTTTTTCTG CAATACTGAA GAAGCTGGAT TTGCAGTACA |
| 1201 | CTGTCCTCAC CACTGAAGAC CATTGCCAGC TTATTGCAAA |
| 1241 | ATGTCCGAAC TTACTAGTTC TCGCGGTGAG GAATGTGATT |
| 1281 | GGAGATAGAG GATTAGGAGT TGTTGCAGAT ACATGCAAGA |
| 1321 | AGCTCCAAAG GCTCAGAATT GAGCGAGGAG ACGATGAAGG |
| 1361 | AGGTGTGCAA GAAGAGCAGG GAGGGGTCTC TCAAGTGGGC |
| 1401 | TTGACGGCTA TAGCCGTCGG TTGCCGTGAA CTGGAATACA |
| 1441 | TAGCTGCCTA TGTGTCTGAT ATAACCAATG GGGCCCTGGA |
| 1481 | ATCTATCGGG ACATTCTGCA AAAAACTCTA TGACTTCCGG |
| 1521 | CTTGTTCTGC TTGATAGAGA AGAGAGGATA ATAGAATTGC |
| 1561 | CACTGGACAA TGGTGTCCGA GCTTTGTTGA GGGGCTGCAC |
| 1601 | CAAACTTCGG AGGTTTGCTC TGTACTTGAG ACCAGGAGGG |
| 1641 | CTCTCAGATG CAGGTCTCGG CTACATTGGA CAGTGCAGTG |
| 1681 | GAAATATCCA ATACATGCTT CTCGGTAATG TTGGGGAAAC |
| 1721 | TGATGATGGA TTGTTCAGTT TCGCATTGGG ATGCGTAAAC |
| 1761 | CTGCGGAAGC TTGAACTCAG GAGTTGTTGC TTCAGCGAGC |
| 1801 | GAGCTCTGGC CCTCGCCATA CTACGCATGC CTTCCCTGAG |
| 1841 | GTACGTATGG GTTCAGGGCT ACAAAGCGTC TCAAATCGGC |
| 1881 | CGAGACCTCA TGCTCATGGC GAGGCCCTTC TGGAACATAG |
| 1921 | AGTTTACATC TCCCAGTTCC GAGAACGCAG GTCGGTTGAT |
| 1961 | GGAAGATGGG GAACCTTGTG TAGATAGTCA TGCTCAGATA |
| 2001 | CTCGCATACC ACTCCCTCGC CGGTAAGAGG TTGGACTGCC |
| 2041 | CACAATCCGT GGTCCCTTTG TATCCTGCCT GAGTGTAAAT |
| 2081 | AGACTAAGCT GGTGTTTTTC TCCCTCATCC CTGCTTCCTT |
| 2121 | AGCCTCCTGG TCAACAAGAA CGATGTTGAT GACTTGATAT |
| 2161 | GTGGTTATTG TATGGATCTA GATGGCTAGC TGCTACGTAC |
| 2201 | TGTAATAAGC TACTAGTAGC TGAGATGTCC TGGAATAAGC |
| 2241 | CCTTGCTATT TTCGCCTGTA CTGCTATCTA ATCCTAGGAA |
| 2281 | GATGTATATT ATTAAGTAAT GGTGGAAGAT GTGAGTCTTG |
| 2321 | CTTGCTCGCC CTGATTTGTA CTATTGGAGG TATAAGAATA |
| 2361 | CCTGGGTTTT TGCCGCCTAC TTTGAGCATT GAGATGTGTC |
| 2401 | T |

A comparison of the Triticum aestivum (wheat) COI1 SEQ ID NO:1 sequence and the Sorghum bicolor COI1 protein with SEQ ID NO:10 sequence is shown below, illustrating that the two proteins have at least 84% sequence identity.

| 84.6% identity in 599 residues overlap; Score: 2632.0; Gap frequency: 1.2% | | |
|---|---|---|
| Seq1 | 1 | MGGEAPEPRRLSRALSL---DGGGVPEEALHLVLGYVDDPRDREAASLACRRWHHIDALT |
| Seq10 | 1 | MGGEAPEPRRLTRALSIGGGDGGWVPEEMLHLVMGFVEDPRDREAASLVCRRWHRVDALS |
| | | *********** **** ** **** **** * * ********** ***** *** |
| Seq1 | 58 | RKHVTVPFCYAVSPARLLARFPRLESLGVKGKPRAAMYGLIPDDWGAYAPPWVAELAAPL |
| Seq10 | 61 | RKHVTVPFCYAVSPARLLARFPRLESLAIKGKPRAAMYGLIPDDWGAYARPWVAELAAPL |
| | | *************************** ******************************* |
| Seq1 | 118 | ECLKALHLRRMVVTDDDLAALVRARGHMLQELKLDKCSGFSTDALRLVARSCRSLRTLFL |
| Seq10 | 121 | ECLKALHLRRMVVTDDDLAELVRARGHMLQELKLDKCTGFSTDGLRLVARSCRSLRTLFL |
| | | ******************* ***************** ***** **************** |
| Seq1 | 178 | EECTITDNGTEWLHDLAANNPVLVTLNFYLTYLRVEPADLELLAKNCKSLISLKISDCDL |
| Seq10 | 181 | EECQINDKGSEWIHDLADGCPVLTTLNFHMTELQVMPADLEFLARSCKSLISLKISDCDV |
| | | *** * * * ** **** *** **** * * * ***** ** ************* |
| Seq1 | 238 | SDLIGFFQIATSLQEFAGAEISEQ----KYGNVKLPSKLCSFGLTFMGTNEMHIIFPFSA |
| Seq10 | 241 | SDLIGFFQFATALEEFAGGTFNEQGELTMYGNVRFPSRLCSLGLTFMGTNEMPIIFPFSA |
| | | ******** ** * **** ** **** ** *** ********** ******* |
| Seq1 | 294 | VLKKLDLQYSFLTTEDHCQLIAKCPNLLVLAVRNVIGDRGLGVVGDTCKKLQRLRVERGE |
| Seq10 | 301 | ILKKLDLQYTVLTTEDHCQLIAKCPNLLVLAVRNVIGDRGLGVVADTCKKLQRLRIERGD |
| | | ********* ******************************** ********** ***** |
| Seq1 | 354 | DDPGMQEEEGGVSQVGLTAIAVGCRELENIAAYVSDITNGAIESIGTFCKNLHDFRIVLL |
| Seq10 | 361 | DEGGVQEEQGGVSQVGLTAIAVGCRELEYIAAYVSDITNGAIESIGTFCKKLYDFRLVLL |
| | | * * *** ******************* ********************* * ******* |
| Seq1 | 414 | DKQETITDLPLDNGARALLMGCTKLRRFALYLRPGGLSDVGLGYIGQHSGTIQYMLLGNV |
| Seq10 | 421 | DREERITELPLDNGVRALLMGCTKLRRFALYLRPGGLSDAGLGYIGQCSGNIQYMLLGNV |
| | | * * ** ****** ************************ ******* ** ********* |
| Seq1 | 474 | GQTDGGLISFAAGCRNLRKLELRSCCFSERALALAIRQMPSLRYVWVQGYRASQTGRDLM |
| Seq10 | 481 | GETDDGLFSFALGCVNLRKLELRSCCFSERALALAILRMPSLRYVWVQGYKASQTGRDLM |
| | | * ** ** *** ** ********************* ************ ********* |
| Seq1 | 534 | LMARPFWNIEFTPPSTETAGRIMEDGEPCVDRQAQVLAYYSLSGKRSDYPQSVVPLYPA |
| Seq10 | 541 | LMARPFWNIEFTPPSSENAGRIMEDGEPCVDSHAQILAYHSLAGKRLDCPQSVVPLYPA |
| | | *************** * ************* ** *** ** *** * ********** |

In another example, an Arabidopsis thaliana COI1 protein with a sequence provided by the NCBI database as accession number 004197.1 (GI:59797640) is shown below as SEQ ID NO:12.

| | |
|---|---|
| 1 | MEDPDIKRCK LSCVATVDDV IEQVMTYITD PKDRDSASLV |
| 41 | CPRWFKIDSE TREHVTMALC YTATPDRLSR RFPNLRSLKL |
| 81 | KGKPRAAMFN LIPENWGGYV TPWVTEISNN LRQLKSVHFR |
| 121 | RMIVSDLDLD RLAKARADDL ETLKLDKCSG FTTDGLLSIV |
| 161 | THCRKIKTLL MEESSFSEKD GKWLHELAQH NTSLEVLNFY |
| 201 | MTEFAKISPK DLETIARNCR SLVSVKVGDF EILELVGFFK |
| 241 | AAANLEEFCG GSLNEDIGMP EKYMNLVFPR KLCRLGLSYM |
| 281 | GPNEMPILFP FAAQIRKLDL LYALLETEDH CTLIQKCPNL |
| 321 | EVLETRNVIG DRGLEVLAQY CKQLKRLRIE RGADEQGMED |
| 361 | EEGLVSQRGL IALAQGCQEL EYMAVYVSDI TNESLESIGT |
| 401 | YLKNLCDFRL VLLDREERIT DLPLDNGVRS LLIGCKKLRR |
| 441 | FAFYLRQGGL TDLGLSYIGQ YSPNVRWMLL GYVGESDEGL |
| 481 | MEFSRGCPNL QKLEMRGCCF SERAIAAAVT KLPSLRYLWV |
| 521 | QGYRASMTGQ DLMQMARPYW NIELIPSRRV PEVNQQGEIR |
| 561 | EMEHPAHILA YYSLAGQRTD CPTTVRVLKE PI |

An example of a nucleotide (cDNA) sequence that encodes the SEQ ID NO:12 COI1 protein (NCBI accession number NM_129552.4 (GI: 1063702813)) is shown below as SEQ ID NO:13.

| | |
|---|---|
| 1 | GCAAAAATGA AAAGAAAAAC ATAGAAGTAG AGAGAAGATC |
| 41 | GCATCTCGAC CGTCAACTTC AGTGTATGAA ATAATGATCG |
| 81 | TCCCACTTGA TCCTCAAAAA TATTATTAAC CAAACAAAAT |
| 121 | TTGATTCCAT CGTCCCACTT TCTTCTTCTT CCTCCCAATC |
| 161 | CGCCTCTTCT TCCTACGCGT GTCTTCTTCT CCCTCACTCT |
| 201 | CTCAATCTCT AGTCTTCTCC GATTCACCGG ATCTTTCCTT |
| 241 | TCTTACTTCT TTCTTCTCAC TCTGGTGGTT ATGTGTGGAT |
| 281 | CTGCGACCTC GATTTCAATT CGAAGTCGTC GGTTTCTTCT |
| 321 | CTAAATCGAA TCTTTCCAGG ATTCGTTTGT TTTTTTCTTT |
| 361 | TGTTTTTTTT TCGATCCGAT GGAGGATCCT GATATCAAGA |
| 401 | GGTGTAAATT GAGCTGCGTC GCGACGGTTG ATGATGTCAT |
| 441 | CGAGCAAGTC ATGACCTATA TAACTGACCC GAAAGATCGC |
| 481 | GATTCGGCTT CTTTGGTGTG TCGGAGATGG TTCAAGATTG |
| 521 | ATTCCGAGAC GAGAGAGCAT GTGACTATGG CGCTTTGCTA |
| 561 | CACTGCGACG CCTGATCGTC TTAGCCGTCG ATTCCCGAAC |
| 601 | TTGAGGTCGC TCAAGCTTAA AGGCAAGCCT AGAGCAGCTA |
| 641 | TGTTTAATCT GATCCCTGAG AACTGGGGAG GTTATGTTAC |
| 681 | TCCTTGGGTT ACTGAGATTT CTAACAACCT TAGGCAGCTC |
| 721 | AAATCGGTGC ACTTCCGACG GATGATTGTC AGTGACTTAG |
| 761 | ATCTAGATCG TTTAGCTAAA GCTAGACCAG ATGATCTTGA |
| 801 | GACTTTGAAG CTAGACAAGT GTTCTGGTTT TACTACTGAT |
| 841 | GGACTTTTGA GCATCGTTAC ACACTGCAGG AAAATAAAAA |
| 881 | CTTTGTTAAT GGAAGAGAGT TCTTTTAGTG AAAAGGATGG |
| 921 | TAAGTGGCTT CATGAGCTTG CTCAGCACAA CACATCTCTT |
| 961 | GAGGTTTTAA ACTTCTACAT GACGCAGTTT GCCAAAATCA |
| 1001 | GTCCCAAAGA CTTGGAAACC ATAGCTAGAA ATTGCCGCTC |
| 1041 | TCTGGTATCT GTGAAGGTCG GTGACTTTGA GATTTTGGAA |
| 1081 | CTAGTTGGGT TCTTTAAGGC TCCAGCTAAT CTTGAAGAAT |
| 1121 | TTTGTGGTGG CTCCTTGAAT GAGGATATTG GAATGCCTGA |
| 1161 | GAAGTATATG AATCTGGTTT TTCCCCGAAA ATTATGTCGG |
| 1201 | CTTGGTCTCT CTTACATGGG ACCTAATGAA ATGCCAATAC |
| 1241 | TATTTCCATT CGCGGCCCAA ATCCGAAAGC TGGATTTGCT |
| 1281 | TTATGCATTG CTAGAAACTG AAGACCATTG TACGCTTATC |
| 1321 | CAAAAGTGTC CTAATTTGGA AGTTCTCGAG ACAAGGAATG |
| 1361 | TAATCGGAGA TAGGGGTCTA GAGGTCCTTG CACAGTACTG |
| 1401 | TAAGCAGTTG AAGCGGCTGA GGATTGAACG CGGTGCAGAT |
| 1441 | GAACAAGGAA TGGAGGACGA AGAAGGCTTA GTCTCACAAA |
| 1481 | GAGGATTAAT CGCTTTGGCT CAGGGCTGCC AGGAGCTAGA |
| 1521 | ATACATGGCG GTGTATGTCT CAGATATAAC TAACGAATCT |
| 1561 | CTTGAAAGCA TAGGCACATA TCTGAAAAAC CTCTGTGACT |
| 1601 | TCCGCCTTGT CTTACTCGAC CGGGAAGAAA GGATTACAGA |
| 1641 | TCTGCCACTG GACAACGGAG TCCGATCTCT TTTGATTGGA |
| 1681 | TGCAAGAAAC TCAGACGATT TGCATTCTAT CTGAGACAAG |
| 1721 | GCGGCTTAAC CGACTTGGGC TTAAGCTACA TCGGACAGTA |
| 1761 | CAGTCCAAAC GTGAGATGGA TGCTGCTGGG TTACGTAGGT |
| 1801 | GAATCAGATG AAGGTTTAAT GGAATTCTCA AGAGGCTGTC |
| 1841 | CAAATCTACA GAAGCTAGAG ATGAGAGGTT GTTGCTTCAG |
| 1881 | TGAGCGAGCA ATCGCTGCAG CGGTTACAAA ATTGCCTTCA |
| 1921 | CTGAGATACT TGTGGGTACA AGGTTACAGA GCATCGATGA |
| 1961 | CGGGACAAGA TCTAATGCAG ATGGCTAGAC CGTACTGCAA |
| 2001 | CATCGAGGTG ATTCCATCAA GAAGAGTCCC GGAAGTGAAT |
| 2041 | CAACAAGGAG AGATAAGAGA GATGGAGCAT CCGGCTCATA |
| 2081 | TATTGGCTTA CTACTCTCTG GGTGGCCAGA GAACAGATTG |
| 2121 | TCCAACAATT GTTAGAGTCC TGAAGGAGCC AATATGATAT |
| 2161 | GACCCAAAAA ATAGGTTTGT ATATAAAGAT TTTTAGTCTC |
| 2201 | GAGTTTTGGG GTTTCCACAA ACTGTGTACT ATACTACTTT |
| 2241 | GGTTCTTTTT TTGTTTCATG TTGTGTCGTC GATGTTTTTG |
| 2281 | GGAGATTACA TAGAGTCAGT CTTGTTTGTT GTATGGTCAT |
| 2321 | TACTTCTTTA TTTTTCCTCA GCGGTCTGTT TACTTTAATT |
| 2361 | TCTTTAATAA AACCCCGAAG ATTTTGAGAG ATTTCTTTAT |
| 2401 | CGTCCATGGT GTTGACTTCT GAGAGCTATA TTTGTTTGGA |
| 2441 | TTGGCATCTG AAATTTTATT TGTGGTTGTG ATTGTTTTGA |
| 2481 | TAACATTAGT AAAAAGGCAA ATAATAGAGT AC |

A comparison of the Triticum aestivum (wheat) COI11 SEQ ID NO:1 sequence and the Arabidopsis thaliana COI1 protein COI1 SEQ ID NO:12 sequence is shown below, illustrating that the two proteins have at least 56% sequence identity.

| 56.1% identity in 570 residues overlap; Score: 1679.0; Gap frequency: 1.6% | | |
|---|---|---|
| Seq1 | 24 | EEALHLVLGYVDDPRDREAASLACRRWHHIDALTRKHVTVPFCYAVSPARLLARFPRLES |
| Seq12 | 18 | DDVIEQVMTYITDPKDRDSASLVCRRWFKIDSETREHVTMALCYTATPDRLSRRFPNLRS |
| | | * * ** ** *** **** ** ** *** ** * ** *** * * |
| Seq1 | 84 | LGVKGKPRAAMYGLIPDDWGAYARPWVAELAAPLECLKALHLRRMVVTDDDLAALVRARG |
| Seq12 | 78 | LKLKGKPRAAMFNLIPENWGGYVTPWVTEISNNLRQLKSVHFRRMIVSDLDLDRLAKARA |
| | | * ******** *** ** * *** * * ** * *** * * ** * ** |
| Seq1 | 144 | HMLQELKLDKCSGFSTDALRLVARSCRSLRTLFLEECTITDNGTEWLHDLAANNPVLVTL |
| Seq12 | 138 | DDLETLKLDKCSGFTTDGLLSIVTHCRKIKTLLMEESSFSEKDGKWLHELAQHNTSLEVL |
| | | * ********* ** * ** ** ** *** ** * * * |
| Seq1 | 204 | NFYLT-YLRVEPADLELLAKNCKSLISLKISDCDLSDLIGFFQIATSLQEFAGAEISE-- |
| Seq12 | 198 | NFYMTEFAKISPKDLETIARNCRSLVSVKVGDFEILELVGFFKAAANLEEFCGGSLNEDI |
| | | *** * * *** * ** ** * * * * *** * * ** * * |
| Seq1 | 261 | ---QKYGNVKLPSKLCSFGLTFMGTNEMHIIFPFSAVLKKLDLQYSFLTTEDHCQLIAKC |
| Seq12 | 258 | GMPEKYMNLVFPRKLCRIGLSYMGPNEMPILFPFAAQIRKLDLLYALLETEDHCTLIQKC |
| | | ** * * *** ** ** *** * *** * **** * * ***** ** ** |
| Seq1 | 318 | PNLLVLAVRNVIGDRGLGVVGDTCKKLQRLRVERGEDDPGMQEEEGGVSQVGLTAIAVGC |
| Seq12 | 318 | PNLEVLETRNVIGDRGLEVLAQYCKQLKRLRIERGADEQGMEDEEGLVSQRGLIALAQGC |
| | | *** ** ********* * ** * *** *** * ** *** *** ** * * ** |
| Seq1 | 378 | RELENIAAYVSDITNGALESIGTFCKNLHDFRLVLLDKQETITDLPLDNGARALLRGCTK |
| Seq12 | 378 | QELEYMAVYVSDITNESLESIGTYLKNLCDFRLVLLDREERITDLPLDNGVRSLLIGCKK |
| | | *** * ******* ****** *** ******** * ********* * ** ** * |
| Seq1 | 438 | LRRFAIYLRPGGLSDVGLGYIGQHSGTIQYMLLGNVGQTDGGLISFAAGCRNLRKLELRS |
| Seq12 | 438 | LRRFAFYLRQGGLTDLGLSYIGQYSPNVRWMLLGYVGESDEGLMEFSRGCPNLQKLEMRG |
| | | ***** *** *** * ** **** * **** ** * ** * ** ** *** * |
| Seq1 | 498 | CCFSERALALAIRQMPSLRYVWVQGYRASQTGRDLMLMARPFWNIEFTPPSTETAGRLME |
| Seq12 | 498 | CCFSERAIAAAVTKLPSLRYLWVQGYRASMTGQDLMQMARPYWNIELIP--SRRVPEVNQ |
| | | ******* * * ***** ******** ** *** **** **** * |
| Seq1 | 558 | DGEPC-VDRQAQVLAYYSLSGKRSDYPQSV |
| Seq12 | 556 | QGEIREMEHPAHILAYYSLAGQRTDCPTTV |
| | | ** * ****** * * * * * |

An example of a COT 1 protein from Brassica rapa (turnip) with NCBI accession number XP_009133392.1 (GI:685284974) has the following sequence (SEQ ID NO:14).

| | |
|---|---|
| 1 | MEDPDIKKCR LSSVTVDDVI EQVMPYITDP KDRDSASLVC |
| 41 | RRWFEIDSET REHVTMALCY TSTPDRLSRR FPNLRSIKLK |
| 81 | GKPRAAMFNL IPENWGGFVT PWVNEIASSL RRLKSVHFRR |
| 121 | MIVSDLDLDV LAKARLDELE ALKLDKCSGF STDGLFSIVK |
| 161 | HCRKMKTLLM EESSFVEKDG NWLHELALHN TSLEVLNFYM |
| 201 | TEFAKINAKD LESIARNCRS LVSVKIGDFE MLELVGFFKA |
| 241 | ATNLEEFCGG SLNEEIGRPE KYMNLTFPPK LCCLGLSYMG |
| 281 | PNEMPILFPF AAQIRKLDLI YALLATEDHC TLIQKCPNLE |
| 321 | VLETRNVIGD RGLEVLGQCC KKLKRLRIER GEDEQGMEDE |
| 361 | EGLVSQRGLV ALAQGCQELE YMAVYVSDIT NESLESIGTY |
| 401 | LKNLCDFRLV LLDQEERITD LPLDNGVRSL LIGCKKLRRF |
| 441 | AFYLRQGGLT DVGLSYIGQY SPNVRWMLLG YVGESDEGLM |
| 481 | EFSPGCPKLQ KLEMRGCCFS ERAIAAAVLK IPSLRYLWVQ |
| 521 | GYRASTTGQD LRLMSRPYWN IELIPARKVP EVNQLGEVRE |
| 561 | MEHPAHILAY YSLAGERTDC PPTVKVLREA |

An example of a nucleotide (cDNA) sequence that encodes the SEQ ID NO:14 Brassica rapa (turnip) COI1 protein (NCBI cDNA accession number XM_009135144.1 (GI:685284973)) is shown below as SEQ ID NO:15.

| | |
|---|---|
| 1 | GCCACTTCTT CCTCCTCTCC TCACGCTCCA CGTCCCCTGC |
| 41 | TAGCATCCCT CCCGCTTCCT CCTCCGATCT CTGCTCGTCT |
| 81 | TATCTTCACT CTCTACTGTA TTACTTTGGA TCTGCGAGAG |
| 121 | ATTCGTGTAA TTGAAATCGA TCTCGTCCCT CAGCTGGTAT |
| 161 | TCGAATTTGT TGATTGTTTT GGTTTGTTTT AGATTCGATT |
| 201 | TCGATTTGTT ACATGGAGGA TCCGGATATC AAGAAGTGCA |
| 241 | GATTGAGCTC CGTGACGGTC GA7GACGTCA TCGAGCAGGT |
| 281 | CATGCCTTAC ATAACCGATC CGAAAGATCG AGACTCCGCT |
| 321 | TCCCTCGTGT GCCGGAGGTG GTTCGAGATC GACTCCGAGA |
| 361 | CGAGGGAGCA CGTGACCATG GCCTTGTGCT ACACCTCGAC |
| 401 | GCCCGATCGT CTCAGCCGTA GGTTTCCCAA TCTGAGGTCG |
| 441 | ATCAAGCTCA AAGGGAAGCC GAGAGCAGCT ATGTTCAATC |
| 481 | TCATCCCCGA GAACTGGGGA GGGTTTGTTA CCCCTTGGGT |
| 521 | CAACGAGATA GCTTCGTCGC TGCGAAGGCT CAAGTCTGTG |
| 561 | CATTTTAGGC GCATGATTGT GAGCGATTTG GATCTGGATG |
| 601 | TTTTGGCTAA GGCGAGGTTG GATGAGCTCG AGGCGTTGAA |
| 641 | GCTTGATAAG TGCTCGGGTT TCTCTAfGGA TGGACTTTTC |
| 681 | AGCATCGTTA AGCACTGCAG GAAAATGAAA ACATTGTTAA |
| 721 | TGGAAGAGAG TTCTTTTGTT GAAAAGGATG GTAACTGGCT |
| 761 | TCATGAACTT GCTCTGCACA ACACTTCTCT CGAGGTTCTA |
| 801 | AATTTCTACA TGACTGAGTT TGCAAAAATC AATGCCAAAG |
| 841 | ACTTGGAAAG CATAGCTAGA AATTGCCGCT CTCTGGTTTC |
| 881 | TGTGAAGATC GGTGACTTTG AGATGTTGGA ACTAGTCGGG |
| 921 | TTCTTTAAAG CTGCAACTAA TCTTGAAGAA TTTTGTGGTG |
| 961 | GCTCCTTAAA TGAAGAAATT GGAAGACCGG AGAAGTATAT |
| 1001 | GAATCTGACT TTCCCTCCAA AACTATGTTG TCTGGGCCTT |
| 1041 | TCTTACATGG GACCTAATGA AATGCCAATA CTGTTTCCAT |
| 1081 | TCGCTCCCCA AATCCGGAAG CTGGATCTGA TCTATGCATT |
| 1121 | GCTCGCAACT GAGGATCATT GTACACTTAT TCAAAAGTGT |
| 1161 | CCTAATTTGG AAGTTCTCGA GAfAAGGAAT GTAATTGGAG |
| 1201 | ATAGGGGTCT AGAGGTTCTT GGACAGTGCT GTAAGAAGTT |
| 1241 | GAAGCGGCTG AGGATTGAAf GGGGTGAAGA TGAACAAGGA |
| 1281 | ATGGAGCATG AAGAAGGCTT AGTCTCACAA AGAGGATTAG |
| 1321 | TCGCTTTGGC TCAGGGCTGC CAGGAGCTAG AATACATGGC |
| 1361 | GGTGTATGTC TCAGATATAA CCAACGAGTC TCTCGAAAGC |
| 1401 | ATAGGCACAT ATCTGAAAAA CCTCTGTGAC TTCCGCCTCG |
| 1441 | TCTTACTCGA CCAAGAAGAG AGAATAACAG ATCTGCCACT |
| 1481 | GGACAATGGA GTCAGATCCC TCTTGATCGG ATGCAAAAAA |
| 1521 | CTCAGACGGT TTGCATTCTA TCTCAGACAA GGCCGCTTAA |
| 1561 | CAGACGTGGG GTTAAGCTAC ATCGGACAGT ACAGTCCAAA |
| 1601 | CGTGAGGTGG ATGCTTCTCG GTTACGTTGG TGAATCAGAC |
| 1641 | GAAGGCCTAA TGGAATTCTC AAGAGGATGT CCGAAACTAC |
| 1681 | AGAAGCTGGA GATGAGAGGT TGTTGCTTCA GCGAGCGAGC |
| 1721 | AATAGCTGCA GCGGTACTGA AAATCCCTTC GCTGAGATAC |
| 1761 | CTGTGGGTAC AAGGCTATAG AGCATCGACG ACGGGACAAG |
| 1801 | ACCTGAGGCT AATGTCTAGA CCGTACTGGA ACATCGAGCT |
| 1841 | GATTCCGGCA AGAAAAGTCC CGGAAGTGAA TCAGCTTGGA |
| 1881 | GAGGTGAGAG AGATGGAGCA TCCTGCTCAT ATACTGGCTT |
| 1921 | GGTTAAAGTC CTGAGGGAGG CATGATGATG ATGATGAAAA |
| 2001 | GCAGGTTTGT ACATAAAGAT TTGGTTTTGA GGTTTCCACG |
| 2041 | AACTGTCGAA TGGATTCTAT TTTTTCTTTA TTGGTGTATT |
| 2081 | GTCTGTAGTT TTGAGAGATT CCATAAAGAC TTTTGAGAGA |
| 2121 | TTGAAATAAG AAGAGAGAAA ACTAGTCTTT CAGAAGA |

A comparison of the Triticum aestivum (wheat) COI1 SEQ ID NO:1 sequence and the Brassica rapa (turnip) COI1 protein SEQ ID NO:14 sequence is shown below, illustrating that the two proteins have at least 56% sequence identity.

| 56.2% identity in 575 residues overlap; Score: 1687.0; Gap frequency: 1.2% | | |
|---|---|---|
| UserSeq1 | 24 | EEALHLVLGYVDDPRDREAASLACRRWHHIDALTRKHVTVPFCYAVSPARLLARFPRLES |
| Seq14 | 17 | DDVIEQVMPYITDPKDRDSASLVCRRWFEIDSETREHVTMALCYTSTPDRLSRRFPNLRS |
| | | * * ***** *** **** ** ** *** ** * ** *** * * |
| UserSeq1 | 84 | LGVKGKPRAAMYGLIPDDWGAYARPWVAELAAPLECLKALHLRRMVVTDDDLAALVRARG |
| Seq14 | 71 | IKLKGKPRAAMFNLIPENWGGFVTPWVNEIASSLRRLKSVHFRRMIVSDLDLDVLAKARL |
| | | ******** *** ** *** * * * ** * *** * * ** * ** |
| UserSeq1 | 144 | HMLQELKLDKCSGFSTDALRLVARSCRSLRTLFLEECTITDNGTEWLHDLAANNPVLVTL |
| Seq14 | 137 | DELEALKLDKCSGFSTDGLFSIVKHCRKMKTLLMEESSFVEKDGNWLHELALHNTSLEVL |
| | | * ************ * ** ** ** *** ** * * * |
| UserSeq1 | 204 | NFYLT-YLRVEPADLELLAKNCKSLISLKISDCDLSDLIGFFQIATSLQEFAGAEISEQ- |
| Seq14 | 197 | NFYMTEFAKINAKDLESIARNCRSLVSVKIGDFEMLELVGFFKAATNLEEFCGGSLNEEI |
| | | *** * *** * ** ** * ** * * *** ** * ** * * |
| UserSeq1 | 262 | ----KYGNVKLPSKLCSFGLTFMGTNEMHIIFPFSAVLKKLDLQYSFLTTEDHCQLIAKC |
| Seq14 | 257 | GRPEKYMNLTFPPKLCCLGLSYMGPNEMPILFPFAAQIRKLDLIYALLATEDHCTLIQKC |
| | | ** * * *** ** ** *** * *** * **** * * ***** ** ** |
| UserSeq1 | 318 | PNLLVLAVRNVIGDRGLGVVGDTCKKLQRLRVERGEDDPGMQEEEGGVSQVGLTAIAVGC |
| Seq14 | 317 | PNLEVLETRNVIGDRGLEVLGQCCKKLKRLRIERGEDEQGMEDEEGLVSQRGLVALAQGC |
| | | *** ** ********* * * **** *** ***** ** *** *** ** * * ** |
| UserSeq1 | 378 | RELENIAAYVSDITNGALESIGTFCKNLHDFRLVLLDKQETITDLPLDNGARALLRGCTK |
| Seq14 | 371 | QELEYMAVYVSDITNESLESIGTYLKNLCDFRLVLLDQEERITDLPLDNGVRSLLIGCKK |
| | | *** * ******* ****** *** ******** * ********* * ** ** * |
| UserSeq1 | 438 | LRRFALYLRPGGLSDVGLGYIGQHSGTIQYMLLGNVGQTDGGLISFAAGCRNLRKLELRS |
| Seq14 | 437 | LRRFAFYLRQGGLTDVGLSYIGQYSPNVRWMLLGYVGESDEGLMEFSRGCPKLQKLEMRG |
| | | ***** *** *** **** **** * **** ** * ** * ** * *** * |
| UserSeq1 | 498 | CCFSERALALAIRQMPSLRYVWVQGYRASQTGRDLMLMARPFWNIEFTPPSTETAGRLME |
| Seq14 | 497 | CCFSERAIAAAVLKIPSLRYLWVQGYRASTTGQDLRLMSRPYWNIELIPARKVPEVNQLG |
| | | ******* * * ***** ******** ** ** ** ** **** * |
| UserSeq1 | 558 | DGEPCVDRQAQVLAYYSLSGKRSDYPQSVVPLYPA |
| Seq14 | 557 | EVRE-MEHPAHILAYYSLAGERTDCPPTVKVLREA |
| | | * ****** * * * * * * * |

An example of a COI1 protein from Brassica napus (rapeseed) with NCBI accession number CDY60996.1 (GI:674872982) has the following sequence (SEQ ID NO:16).

| | |
|---|---|
| 1 | MLQRIFWMFF FSFNMLTRYF IKTPPGYFCR LARCAAYATR |
| 41 | LTKQTDSIAS SPPSIYIKNN NYPLCPLDPK LLLLLSTLLI |
| 81 | PSFTHTYATS SSSPHAPQIR VIEIDLIRFR FVTMEDPDIK |
| 121 | KCRLSSVTVD DVIEQVMPYI TDPKDRDSAS LVCRRWFEID |
| 161 | SETREHVTMA LCYTSTPDRL SRRFPNLRSI KLKGKPRAAM |
| 201 | FNLIPENWGG FVTPWVNEIA SSLRRLKSVH FRRMIVSDLD |
| 241 | LDVLAKARLD ELEALKLDKC SGFSTDGLFS IVKHCRKMKT |
| 281 | LLMEESSFVE KDGNWLHELA LHNTSLEVLN FYMTEFAKIN |
| 321 | AKDLESIARN CRSLVSVKIG DFEMLELVGF FKAATNLEEF |
| 361 | CGGSLNEEIG RPEKYMNLTF PPKLCCLGLS YMGPNEMPIL |
| 401 | FPFAAQIRKL DLIYALLATE DHCTLIQKCP NLEVLETRNV |
| 441 | IGDRGLEVLG QCCKKLKRLR IERGEDEQGM EDEEGLVSQR |
| 481 | GLVALAQGCQ ELEYMAVYVS DITNESLESI GTYLKNLCDF |
| 521 | RLVLLDQEER ITDLPLDNGV RSLLIGCKKL RRFAFYLRQS |
| 561 | GLTDVGLSYI GQYSPNVRWM LLGYVGESDE GLMEFSRGCP |
| 601 | KLQKLEMRGC CFSERAIAAA VLKIPSLRYL WVQGYRASTT |
| 641 | GQDLRLMSRP YWNIELIPAR KVPEVNQLGE VREMEHPAHI |
| 681 | LAYYSLAGER TDCPPTVKVL REA |

A comparison of the Triticum aestivum (wheat) COI1 SEQ ID NO:1 sequence and the Brassica napus (rapeseed) COI1 protein SEQ ID NO:16 sequence is shown below, illustrating that the two proteins have at least 56% sequence identity.

| 56.0% identity in 575 residues overlap; Score: 1681.0; Gap frequency: 1.2% | | |
|---|---|---|
| Seq1 | 24 | EEALHLVLGYVDDPRDREAASLACRRWHHIDALTRKHVTVPFCYAVSPARLLARFPRLES |
| Seql6 | 130 | DDVIEQVMPYITDPKDRDSASLVCRRWFEIDSETREHVTMALCYTSTPDRLSRRFPNLRS |
| | | * * ** ** *** **** ** ** *** ** * ** *** * * |
| Seq1 | 84 | LGVKGKPRAAMYGLIPDDWGAYARPWVAELAAPLECLKALHLRRMVVTDDDLAALVRARG |
| Seql6 | 190 | IKLKGKPRAAMFNLIPENWGGFVTPWVNEIASSLRRLKSVHFRRMIVSDLDLDVLAKARL |
| | | ******** *** ** *** * * * ** * *** * * ** * ** |
| Seq1 | 144 | HMLQELKLDKCSGFSTDALRLVARSCRSLRTLFLEECTITDNGTEWLHDLAANNPVLVTL |
| Seql6 | 250 | DELEALKLDKCSGFSTDGLFSIVKHCRKMKTLLKEESSFVEKDGNWLHELALHNTSLEVL |
| | | * ************ * ** ** ** *** ** * * * |
| Seq1 | 204 | NEYLT-YLRVEPADLELLAKNCKSLISLKISDCDLSDLIGFFQIATSLQEFAGAEISEQ- |
| Seq16 | 310 | NEYMTEFAKINAKDLESIARNCRSLVSVKIGDFFMLELVGFFKAATNLEEFCGGSLNEEI |
| | | *** * *** * ** ** * ** * * *** ** * ** * * |
| Seq1 | 262 | ----KYGNVKLPSKLCSFGLTFMGTNEMHIIFPFSAVLKKLDLQYSFLTTEDHCQLIAYC |
| Seq16 | 370 | GRPEKYMNLTFPPKLCCLGLSYMGPNEMPILFPFAAQIRKLDLTYALLATEDHCTLIQKC |
| | | ** * * *** ** ** *** * *** * **** * * ***** ** ** |
| Seq1 | 318 | PNLLVLAVRNVIGDRGLGVVGDTCKKLQRLRVERGEDDPGMQEEEGGVSQVGLTAIAVGC |
| Seq16 | 430 | PNLEVLETRNVIGDRGLEVLGQCCKKLKRLRIERGEDEQGMEDEEGLVSQRGLVALAQGC |
| | | *** ** ********* * * **** *** ***** ** *** *** ** * * ** |
| Seq1 | 378 | RELENIAAYVSDITNGALESIGTFCKNLHDFRLVLLDKQETITDLPLDNGARALLRGCTK |
| Seq16 | 490 | QELEYMAVYVSDITNESLESIGTYLKNLCDFRLVLLDQEERITDLPLDNGVRSLLIGCKK |
| | | *** * ******* ****** *** ******** * ********* * ** ** * |
| Seq1 | 438 | LRRFALYLRPGGLSDVGLGYIGQHSGTIQYMLLGNVGQTDGGLISFAAGCRNLRKLELRS |
| Seq16 | 550 | LRRFAFYLRQSGLTDVGLSYIGQYSPNVRWMLLGYVGESDEGLMEFSRGCPKLQKLEMRG |
| | | ***** *** ** **** **** * **** ** * ** * ** * *** * |
| Seq1 | 498 | CCFSERALALAIRQMPSLRYVWVQGYRASQTGRDLMLMARPFWNIEFTPPSTETAGRLME |
| Seq16 | 610 | CCFSERAIAAAVIKIPSLRYLWVQGYRASTTGQDLRLMSRPYWNIELIPARKVPEVNQLG |
| | | ******* * * ***** ******** ** ** ** ** **** * |
| Seq1 | 558 | DGEPCVDRQAQVLAYYSLSGKRSDYPQSVVPLYPA |
| Seq16 | 670 | EVRE-MEHPAHTLAYYSLAGERTDCPPTVKVLREA |
| | | * ****** * * * * * * * |

An example of a COI1 protein from Brassica oleracea (cabbage, Brussel sprouts, kale, cauliflower, etc.) with NCBI accession number XP_013628733.1 (GI:922451771) has the following sequence (SEQ ID NO:17).

| | |
|---|---|
| 1 | MTMEDPDIKK CRLSSVTVDD VIEQVMPYIT DPKDRDSASL |
| 41 | VCRRWFEIDS ETREHVTMAL CYTSTPDRLS RRFPNLRSIK |
| 81 | LKGKPRAAMF NLIPENWGGF VTPWVNEVAS SLPRLKSVHF |
| 121 | RRMIVSDLDL DVLAKARLDE LEALKLDKCS GESTDGLFSI |
| 161 | VKHCRKMKTL LMEESSFVEK DGNWLHELAL HNTSLEVLNF |
| 201 | YMTEFAKINA KDLESIARNC RSLVSVKIGD FEMLELVGFF |
| 241 | KAATNLEFFC GGSFNEEIGR PEKYMNLTFP PKLCCLGLSY |
| 281 | MGPNEMPILF PFAAQIRKLD LIYALLATED HCTLIQKCPN |
| 321 | LEVLETRNVI GDRGLEVLGQ CCKKLKRLRI ERGEDEQGME |
| 361 | DEEGLVSQRG LVALAQGCQE LEYMAVYVSD ITNESLESIG |
| 401 | TYLKNLCDFR LVLLDQEERI TDLPLDNGVR SLLIGCKKLR |
| 441 | RFAFYLRQGG LTDVGLSYIG QYSPNVRWML LGYVGESDEG |
| 481 | LMEFSRGCPK LQKLEMRGCC FSERAIAAAV LKIPSLRYLW |
| 521 | VQGYRASTTG QDLRLMSRPY WNIELIPARK VPEVNQLGEV |
| 561 | REMEHPAHIL AYYSLAGERT DCPPTVKVLR EA |

An example of a nucleotide (cDNA) sequence that encodes the Brassica oleracea SEQ ID NO:17 COI1 protein (NCBI cDNA accession number XM_013773279.1 (GI:922451770)) is shown below as SEQ ID NO:18.

| | |
|---|---|
| 1 | ATTATTATTA TCAACACTTT TGATTCCTTC CTCCACACAC |
| 41 | ACTCACGCCA CTTCTTCCTC CTCTCCTCAC GCTCCACCTA |
| 81 | TCGTGATTCC TATACTCGAT TTCGATTTGT TATCCGTTTG |
| 121 | TTTGATGACG ATGGAGGATC CGGATATCAA GAAGTGCAGA |
| 161 | TTGAGGTCCG TGACGGTCGA TGAGGTCATC GAGCAGGTCA |
| 201 | TGCCTTACAT AACCGATCCG AAAGATCGAG ACTCCGCTTC |
| 241 | CCTCGTGTGC CGGAGGTGGT TCGAGATCGA CTCCGAGACG |
| 281 | AGCGAGCACG TGACCATGGG ACTATGGTAC ACCTCGACTC |
| 321 | CTGACCGTCT CAGCCGTAGG TTTCCGAATC TGAGGTCGAT |
| 361 | TAAGCTCAAA GGGAAGCCGA GAGCAGCTAT GTTCAATCTC |
| 401 | ATCCCCGAGA ACTGGGGAGG GTTTGTTATC CCTTGGGTCA |
| 441 | ACGAGGTAGC TTCATCTCTG CCAAGGCTCA AGTCTGTGCA |
| 481 | TTTTAGGCGG ATGATTGTCA GCGATTTGGA TCTTGATGTT |
| 521 | TTGGCTAAGG CGAGGTTGGA TGAGGTCGAG GCGTTGAAGG |
| 561 | TCGATAAGTG CTCAGCTTTC TCTACGGATG GACTTTTCAG |
| 601 | CATCGTTAAG CACTGCAGGA AAATGAAAAC ATTGTTAATG |
| 641 | GAAGAGAGTT CTTTTGTTGA AAAGGATGGT AACTGGCTGC |
| 681 | ATGAACTTGC TCTGCACAAC ACTTCTCTTG AGGTTCTAAA |
| 721 | TTTCTACATG ACTGAGTTTG CAAAAATCAA TGCCAAAGAC |
| 761 | TTGGAAAGGA TAGCTAGAAA TTGCCGGTCT CTGGTTTCTG |
| 801 | TGAAGATCGG TGACTTTGAG ATGTTGGAAC TAGTCGGGTT |
| 841 | CTTTAAAGCT GCAACTAATC TTGAAGAATT TTGTGGCGGC |
| 881 | TCCTTCAATG AAGAAATTGG AAGACCGGAG AAGTATATGA |
| 921 | ATCTGACTTT CCCTCCAAAA CTATGTTGTC TTGGCCTTTC |
| 961 | TTACATGGGA CCTAATGAAA TGCCAATACT GTTTCCATTC |
| 1001 | GCTGCCCAAA TCCGGAAGCT GGATCTGATC TATGCATTGC |
| 1041 | TCGCAACTGA GCATCATTGT ACACTTATTC AAAAGTGTCC |
| 1081 | TAATTTGGAA GTTCTCGAGA CAAGGAATGT AATTGGAGAT |
| 1121 | AGGGGTCTAG AGGTTCTTGG ACAGTGCTGT AAGAAGTTGA |
| 1161 | AGCGGCTGAG GATTGAACGG GGTGAAGATG AACAAGGAAT |
| 1201 | GGAGGATGAA GAAGGCCTAG TATCACAAAG AGGATTAGTC |
| 1241 | GCTTTGGCTC AGGGCTGCCA GGAGCTAGAA TACATGGCGG |
| 1281 | TGTATGTCTC AGATATAACC AACGAGTCTC TCGAAAGGAT |
| 1321 | AGGCACATAT CTGAAAAACC TCTGTGACTT CCGCCTCGTC |
| 1361 | TTACTCGACC AAGAAGAGAG AATAACAGAT CTGCCACTAG |
| 1401 | ACAACGGAGT CCGATCCCTC TTGATCGGAT GCAAGAAACT |
| 1441 | CAGACGGTTT GCATTCTATC TCAGACAAGG CGGCTTAACA |
| 1481 | GACGTGGGGT TAAGCTACAT CGGACAGTAC AGTCCAAACG |
| 1521 | TGAGGTGGAT GCTTCTCGGT TACGTTGGTG AATCAGACGA |
| 1561 | AGGCCTAATG GAGTTCTCAA GAGGATGTCC GAAACTACAG |
| 1601 | AAGGTGGAGA TGAGAGGTTG TTGCTTCAGC GAGCGAGCAA |
| 1641 | TAGGTGCAGC GGTACTGAAA ATCCCTTCGC TGAGATATCT |
| 1681 | GTGGGTACAA GGCTATAGAG CATCAATGAC GGGACAAGAC |
| 1721 | CTGAGGCTAA TGTCTAGACC GTACTGGAAC ATCGAGCTGA |
| 1761 | TTCCGGCAAG AAAAGTCCCA GAAGTGAATC AGCTTGGAGA |
| 1801 | GGTGAGAGAG ATGGAGCATC CTGCTCATAT ACTGGCTTAC |
| 1841 | TACTCTCTGG CTGGTGAGAG AACAGATTGT CCACCAACTG |
| 1881 | TTAAAGTCCT GAGGGAGGCA TGATGATGAT GATGATGATG |
| 1921 | ATGAAAAGCA GGTTTGTACA TAAAGATTTG GTTTTGAGGT |
| 1961 | TTCCACGAAC TGTCGAATGG ATTCTATTTT TCTTTATTGG |
| 2001 | TGTATTGTCT GTAGTTTTGA GAGATTCCAT AAAGACTTTT |
| 2041 | GAGAGATTGA AATAAGAAGA GAGAAAACTA GTCTATTCAG |
| 2081 | AAGA |

A comparison of the Triticum aestivum (wheat) COI1 SEQ ID NO:1 sequence and the Brassica oleracea (cabbage, Brussel sprouts, kale, cauliflower, etc.) COI1 protein SEQ ID NO:17 sequence is shown below, illustrating that the two proteins have at least 56% sequence identity.

| 56.2% identity in 575 residues overlap; Score: 1683.0; Gap frequency: 1.2% | | |
|---|---|---|
| Seq1 | 24 | EEALHLVLGYVDDPRDREAASLACRRWHHIDALTRKHVTVPFCYAVSPARLLARFPRLES |
| Seq17 | 19 | DDVIEQVMPYITDPKDRDSASLVCRRWFEIDSETREHVTMALCYTSTPDRLSRRFPNLRS |
| | | * * ** ** *** **** ** ** *** ** * ** *** * * |
| Seq1 | 84 | LGVKGKPRAAMYGLIPDDWGAYARPWVAELAAPLECLKALHLRRMVVTDDDLAALVRARG |
| Seq11 | 79 | IKLKGKPRAAMFNLIPENWGGFVTPWVNEVASSLPRLKSVHFRRMIVSDLDLDVLAKARL |
| | | ******** **** ** *** * * * *** * * ** ** |
| Seq1 | 144 | HMLQELKLDKCSGFSTDALRLVARSCRSLRTLFLEECTITDNGTEWLHDLAANNPVLVTL |
| Seq17 | 139 | DELEALKLDKCSGFSTDGLFSIVKHCRKMKTLLMEESSFVEKDGNWLHELALHNTSLEVL |
| | | * ************ * ** ** ** *** ** * * * |
| Seq1 | 204 | NFYLT-YLRVEPADLELLAKNCKSLISLKISDCDLSDLIGFFQIATSLQEFAGAEISEQ- |
| Seq17 | 199 | NFYMTEFAKINAYDLESIARNCRSLVSVKIGDFEMLELVGFFKAATNLEEFCGGSFNEEI |
| | | *** * *** * ** ** * ** * * *** ** * ** * * |
| Seq1 | 262 | ----KYGNVKLPSKLCSFGLTFMGTNEMHIIFPFSAVLKKLDLQYSFLTTEDHCQLIAKC |
| Seq17 | 259 | GRPEKYMNLTFPPKLCCLGLSYMGPNEMPILFPFAAQIRKLDLIYALLATEDHCTLIQKC |
| | | ** * * *** ** ** *** * *** * **** * * ***** ** ** |
| Seq1 | 318 | PNLLVLAVRNVIGDRGLGVVGDTCKKLQRLRVERGEDDPGMQEEEGGVSQVGLTAIAVGC |
| Seq17 | 319 | PNLEVLETRNVIGDRGLEVLGQCCKKLKRLRIERGEDEQGMEDEEGLVSQRGLVALAQGC |
| | | *** ** ********* * * **** *** ***** ** *** *** ** * * ** |
| Seq1 | 318 | RELENIAAYVSDITNGALESIGTFCKNLHDFRLVLLDKQETITDLPLDNGARLLLRGCTK |
| Seq11 | 379 | QELEYMAVYVSDITNESLESIGTYLKNLCDFRLVLLDQEERITDLPLDNGVRSLLIGCKK |
| | | *** * ******** ****** *** ******** * ********* * ** ** * |
| Seq1 | 438 | LRRFALYLRPGGLSDVGLGYIGQHSGTIQYMLLGNVGQTDGGLISFAAGCRNLRKLELRS |
| Seq17 | 439 | LRRFAFYLRQGGLTDVGLSYIGQYSPNVRWMLLGYVGESDEGLMEFSRGCPKLQKLEMRG |
| | | ***** *** *** **** **** * **** ** * ** * ** * *** * |
| Seq1 | 498 | CCFSERALALAIRQMPSLRYVWVQGYRASQTGRDLMLMARPFWNIEFTPPSTETAGRLME |
| Seq17 | 499 | CCFSERAIAAAVLKIPSLRYLWVQGYRASTTGQDLRLMSRPYWNIELIPARKVPEVNQLG |
| | | ******* * * ***** ******** ** ** ** ** **** * |
| Seq1 | 558 | DGEPCVDRQAQVLAYYSLSGKRSDYPQSVVPLYPA |
| Seq17 | 559 | EVRE-MEHPAHILAYYSLAGERTDCPPTVKVLREA |
| | | * ****** * * * * * * * |

An example of a COI1 protein from Theobroma cacao (cocoa) with NCBI accession number XP_007009091.2 (GI: 1063526274) has the following sequence (SEQ ID NO:19).

| | |
|---|---|
| 1 | MEENDNKMNK TMTSPVGMSD VVLGCVMPYI HDPKDRDAVS |
| 41 | LVCRRWYELD ALTRKHITIA LCYTTSPDRL RRRFQHLESL |
| 81 | KLKGKPRAAM FNLIPEDWGG YVTPWVNEIA ENFNCLKSLH |
| 121 | FRRMIVKDSD LEVLARSRGK VLQVLKLDKC SGFSTDGLLH |
| 161 | VGRSCRQLKT LFLEESLIVE KDGQWLHELA VNNSVMETLN |
| 201 | FYMTDLVKVS FEDLELIARN CRNLASVKIS DCEILDLVGF |
| 241 | FPAAAVLEEF CGGSFNEQPD RYHAVSFPPK LCRLGLTYMG |
| 281 | KNEMPIVFPF ASLLKKLDLL YALLDTEDHC LLIQRCPNLE |
| 321 | VLETRNVIGD RGLEVLARSC KRLKRLRIER GADEQGMEDE |
| 361 | EGVVSQRGLM ALAQGCLELE YLAVYVSDIT NASLEYIGTY |
| 401 | SKNLSDFRLV LLDREERITD LPLDNGVRAL LRGCEKLRRF |
| 441 | ALYLRPGGLT DVGLSYIGQY SPNVRWMLLG YVGESDAGLL |
| 481 | EFSKGCPSLQ KLEMRGCCFS EHALAVTVMQ LTSLRYLWVQ |
| 521 | GYRASQSGRD LLAMARPFWN IELIPARRVV MNDQVGEAVV |
| 561 | VEHPAHILAY YSLAGPRTDF PETVIPLDPL VAA |

An example of a nucleotide (cDNA) sequence that encodes the Theobroma cacao SEQ ID NO:19 COI1 protein (NCBI cDNA accession number XM_007009029.2 GI:1063526273)) is shown below as SEQ ID NO:20.

| | |
|---|---|
| 1 | AAGTTTCAGC TCTCCTTCTC TGTTTCACGT TTCTGTGGGC |
| 41 | GGTCTCTACT CTGCCATGCC TTCTCTACAC GACCCATTTT |
| 81 | TGACCCGATT CGTTTAGCCC CGGGGGAAAT TTGCTTCGTT |
| 121 | TCAGATCCTA CCGCCGTTTC GTTTCTTCCA CTTCCGTAAA |
| 161 | AGAGAAGAGT TCCACGCCCG TTTCTTCTTC TTCTTCTTCT |
| 201 | TCAGATCAGT CTTTTTTTTT TTTTGCCGTT TCGCGTTTCT |
| 241 | GGTTTATTTG GGCTGAAAAG ATCCGATTCG ATTGTATTGA |
| 281 | ATGGAGGAAA ATGATAACAA GATGAATAAA ACGATGATGT |
| 321 | CACCAGTCGG TATGTCGGAT GTCGTTTTAG GCTGCGTGAT |
| 361 | GCCGTACATC CACGACCCGA AAGACCGGCA CGCAGTTTCG |
| 401 | CTCGTGTGCC GACGTTGGTA CGAGCTCGAC GCGTTGACGA |
| 441 | GGAAGCACAT AACGATCGCG CTTTGCTACA CGACGAGTCC |
| 481 | CGATCGGTTG CGATGTCGTT TCCAGCACTT GGAATCTTTG |
| 521 | AAGTTGAAAG GCAAGCCTCG GGCGGCGATG TTCAATTTGA |
| 561 | TATCTGAGGA TTGGGGAGGG TACGTGACGC CGTGGGTGAA |
| 601 | TGAGATAGCT GAGAATTTTA ATTGCTTGAA ATCTTTGCAT |
| 641 | TTTAGAAGGA TGATTGTTAA AGATTCGGAT CTGGAAGTTT |
| 681 | TGGCTCGGTC TAGAGGGAAG GTTTTGCAGG TTTTGAAGCT |
| 721 | TGATAAATGC TCTGGTTTCT CTACTGATGG TCTCTTGCAT |
| 761 | GTTGGATGCT CCTGCCGGCA ATTAAAAATC TTGTTCCTGG |
| 801 | AAGAGAGGTT AATTGTTGAG AAAGATGGTC AATGGCTTCA |
| 841 | TGAGCTTGCA GTAAATAACT CAGTTATGGA GACTTTGAAC |
| 881 | TTTTATATGA CAGATCTTGT CAAAGTGAGT TTTGAAGACC |
| 921 | TTGAACTTAT TGCTAGAAAT TGTCGCAACT TGGCCTCTGT |
| 961 | GAAAATTAGC GATTGTGAAA TTTTGGATCT TGTTGGTTTC |
| 1001 | TTTCCTGCTG CTGCTGTTTT AGAAGAATTT TGTGGTGGTT |
| 1041 | CTTTCAATGA GCAACCGCAT AGGTACCATG CTGTATCATT |
| 1081 | CCCCCCAAAG TTATGCCGTT TGGGTTTAAC ATACATGGGG |
| 1121 | AAGAATGAAA TGCCAATTGT GTTCCCTTTT GCATCCTTGC |
| 1161 | TTAAAAAGTT GGATCTCCTC TATGCATTAC TTGACACAGA |
| 1201 | AGACCACTGC TTGTTAATTC AGAGATGCCC CAACTTAGAA |
| 1241 | GTTCTTGAGA CAAGGAATGT TATTGGAGAT AGAGGATTAG |
| 1281 | AAGTTCTTGC TCGAAGTTGT AAGAGACTAA AGAGGCTTAG |
| 1321 | AATTGAAAGG GGTGCTGATC AGCAGGCAAT GGAGGATGAA |
| 1361 | GAAGGTGTGG TTTCACAAAG AGGATTAATG GCTTTAGCTC |
| 1401 | AGGGATGCCT TGAATTGGAA TACTTGGCTG TTTATGTATC |
| 1441 | TGACATCACC AATGCATCAT TGGAATACAT TGGGACTTAC |
| 1481 | TCAAAAAATC TCTCTGATTT TCGCCTAGTC TTGCTTGACC |
| 1521 | GAGAAGAAAG GATAATAGAT TTGCCTCTTG ATAATGGAGT |
| 1561 | CCGGGCTCTA TTGAGGGGCT GTGAAAAGCT TAGAAGATTT |
| 1601 | GCTCTGTACC TCCGACCTGG TGGTTTGACT GATGTAGGCC |
| 1641 | TCAGTTATAT TGGGCAATAC AGTCCGAATG TAAGATGGAT |
| 1681 | GCTTCTAGGT TATGTTGGGG AGTCGGATGC CGGGCTTTTG |
| 1721 | GAGTTCTCTA AGGGATGCCC AAGCCTGCAG AAACTAGAAA |
| 1761 | TGAGGGGTTG TTGCTTCAGT GAGCATGCAC TTGCAGTTAT |
| 1801 | TGTGATGCAA TTAACTTCCT TGAGGTATTT GTGGGTGCAA |
| 1841 | GGATATAGAG CGTCACAATC AGGTCGTGAT CTTTTAGCAA |
| 1881 | TGGCTCGTCC ATTTTGGAAT ATCGAGCTAA TTCCTGCAAG |
| 1921 | ACGAGTAGTT ATGAATGATC AGGTTGGAGA GGCTGTTGTG |
| 1961 | GTTGAGCATC CGGCTCATAT ACTCGCGTAT TACTCCCTAG |
| 2001 | CTGGACCAAG AACAGATTTT CCAGAAACTG TTATTCCTTT |
| 2041 | GGATCCATTA GTTGCTGCGT AGAGCTGTAA ATATGACCTA |
| 2081 | TTTTTCGAAG TGTCCATTTT TCCCATCCAC GTTCTGTCTA |
| 2121 | TAAAGTTTCT GCACCTTTCT CTTTTCTCTT TTCCTTTCCT |
| 2161 | TTTTGTTTAG AGGGTTTCCA ATTTGATATT TCATTTTCGA |
| 2201 | TTTTATTTCT AGATTTTGTC CTGTAATAAG ATTGTGTTTT |
| 2241 | CTTCTGTAAT TTTGAAAGCA CTTGCACTCT TGGTGGGCTA |
| 2281 | CTGTTTTTGT CCCTTGTCCC TGCAAAAAGT AGTGAATGAC |
| 2321 | TCTTAACGCA ATA |

A comparison of the Triticum aestivum (wheat) COI1 SEQ ID NO:1 sequence and the Theobroma cacao (cocoa) COI1 protein SEQ ID NO:19 sequence is shown below, illustrating that the two proteins have at least 61% sequence identity.

| 61.0% identity in. 574 residues overlap; Score: 1840.0; Gap frequency: 0.7% | | |
|---|---|---|
| Seq1 | 21 | GVPEEALHLVLGYVDDPRDREAASLACRRWHHIDALTRKHVTVPFCYAVSPARLLARFPR |
| Seq19 | 17 | GMSDVVLGCVMPYIHDPKDRDAVSLVCRRWYELDALTRKHITIALCYTTSPDRIRRRFQH |
| | | * * * ** ** * ** **** ******* * ** ** * ** |
| Seq1 | 81 | LESLGVKGKPRAAMYGLIPDDWGAYARPWVAELAAPLECLKALHLRRMVVTDDDLAALVR |
| Seq19 | 77 | LESLKLKGKPRAAMFNLIPEDWGGYVTPWVNEIAENFNCLKSLHFRRMIVKDSDLEVLAR |
| | | *** ******** ******* *** ** *** ** *** * ** *** |
| Seq1 | 141 | ARGHMLQELKLDKCSGFSTDALRLVARSCRSLRTLFLEECTITDNGTEWLHDLAANNPVL |
| Seql9 | 137 | SRGKVLQVLKLDKCSGFSTDGLLHVGRSCRQLKTLFLEESLIVEKDGQWLHELAVNNSVM |
| | | ** ** ************** * **** * ****** * *** ** ** |
| Seq1 | 201 | VTLNFYLTYL-RVEPADLELLAKNCKSLISLKISDCDLSDLIGFFQIATSLQEFAGAEIS |
| Seq19 | 197 | ETLNFYMTDLVKVSFEDLELIARNCRNLASVKISDCEILDLVGFFPAAAVLEEYCGGSFN |
| | | ***** * * * **** * ** * * ***** ** *** * * * * |
| Seq1 | 260 | EQ--KYGNVKLPSKLCSEGLTFMGTNEMHIIFPFSAVLKKLDLQYSFLTTEDHCQLIAKC |
| Seq19 | 257 | EQPDRYHAVSFPPKLCRLGLTYMGKNEMPIVFPFASLLKKLDLLYALLDTEDHCLLIQRC |
| | | ** * * * *** *** ** *** * *** ****** * * ***** ** * |
| Seq1 | 318 | PNLLVLAVRNVIGDRGLGVVGDTCKKLQRIRVERGEDDPGMQEEEGGVSQVGLTAIAVGC |
| Seq19 | 317 | PNLEVLETRNVIGDRGLEVLARSCKRLKRLRIERGADEQGMEDEEGVVSQRGLMALAQGC |
| | | *** ** ********* * ** * *** ***** ** *** ** ** * * ** |
| Seq1 | 318 | RELENIAAYVSDITNGALESIGTECKNLHDFRLVLLDKQETITDLPLDNGARALLRGCTK |
| Seq19 | 311 | LELEYLAVYVSDITNASLEYIGTYSKNLSDFRLVLLDREERITDLPLDNGVRALLRGCEK |
| | | *** * ******* ** *** *** ******** * ********* ******* * |
| Seq1 | 438 | LRRFALYLRPGGLSDVGLGYIGQHSGTIQYMLLGNVGQTDGGLISFAAGCHNLRKLELRS |
| Seql9 | 437 | LRRFALYLRPGGLTDVGLSYIGQYSPNVRWMLLGYVGESDAGLLEFSKGCPSLQKLEMRG |
| | | ************* **** **** * **** ** ** * ** * *** * |
| Seq1 | 498 | CCFSERALALAIRQMPSLRYVWVQGYRASQTGRDLMLMARPFWNIEFTPPSTETAGRLME |
| Seq19 | 497 | CCFSEHALAVTVMQLTSLRYLWVQGYRASQSGRDLLAMARPFWNIELIPARRVVMNDQVG |
| | | ***** *** * **** ********* **** ********* * |
| Seq1 | 558 | DGEPCVDRQAQVLAYYSLSGKRSDYPQSVVPLYP |
| Seq19 | 557 | EAV-VVEHPAHILAYYSLAGPRTDFPETVIPLDP |
| | | * ****** * * * * * ** * |

An example of a COI1 protein from Glycine max (soybean) with NCBI accession number NP_001238590.1 (GI:351724347) has the following sequence (SEQ ID NO:21).

| | |
|---|---|
| 1 | MTEDRNVRKT RVVDLVLDCV IPYIDDPKDR DAVSQVCRRW |
| 41 | YELDSLTRKH VTIALCYTTT PARLRRRFPH LESLKLKGKP |
| 81 | RAAMFNLIPE DWGGHVTPWV KEISQYFDCL KSLHFRRMIV |
| 121 | KDSDLRNLAR DRGHVLHSLK LDKCSGFTTD GLFHIGRFCK |
| 161 | SLRVLFLEES SIVEKDGEWL HFLALNNTVL ETLNFYLTDI |
| 201 | AVVKIQDLEL LAKNCPNLVS VKLTDSEILD LVNFFKHASA |
| 241 | LEEFCGGTYN EEPEKYSAIS LPAKLCRLGL TYIGKNFEPI |
| 281 | VFMFAAVLKK LDLLYAMLDT EDHCMLIQKC PNLEVLETRN |
| 321 | VIGDRGLEVL GRCCKRLKRL RIERGDDDQG MEDEEGTVSH |
| 361 | RGLIALSQGC SELEYMAVYV SDITNASLEH IGTHLKNLCD |
| 401 | FRLVLLDHEE KITDLPLDNG VRALLRGCNK LRRFALYLRR |
| 441 | GGLTDVGLGY IGQYSPNVPW MLLGYVGESD AGLLEFSKGC |
| 481 | PSLQKLEMRG CSFFSERALA VAATQLTSLR YLWVQGYGVS |
| 521 | PSGRDLLAMA RPFWNIELIP SRKVAMNTNS DETVVVEHPA |
| 561 | HILAYYSLAG QRSDFPDTVV PLDTATCVDT |

An example of a nucleotide (cDNA) sequence that encodes the Glycine max SEQ ID NO:21 COI1 protein (NCBI cDNA accession number NM_001251661.1 (GI:351724346)) is shown below as SEQ ID NO:22.

| | |
|---|---|
| 1 | ATGACGGAGG ATCGGAATGT GCGGAAGATA CGTGTGGTCG |
| 41 | ACCTGGTCCT CGATTGTGTC ATCCCTTACA TCGACGACCC |
| 81 | CAAGGATCGC GACGCCGTCT CACAGGTCTG CCGACGCTGG |
| 121 | TACGAACTCG ACTCCCTCAC TCGGAAGCAC GTCACCATCG |
| 161 | CCCTCTGCTA CACCACCACG CCGGCGCGCC TCCGCCGCCG |
| 201 | CTTCCCGCAC CTTGAGTCGC TCAAGCTCAA GGGCAAGCCC |
| 241 | CGAGCAGCAA TGTTCAACTT GATACCCGAG GATTGGGGAG |
| 281 | GCCATGTCAC CCCATGGGTC AAGGAGATTT CTCAGTATTT |
| 321 | CGATTGCCTC AAGAGTCTCC ACTTCCGCCG TATGATTGTC |
| 361 | AAAGATTCCG ATCTTCGCAA TCTCGCTCGT GACCGCGGCC |
| 401 | ACGTGCTTCA CTCTCTCAAG CTTGACAAGT GCTCCGGTTT |
| 441 | CACCACCGAT GGTCTTTTCC ATATCGGTCG CTTTTGCAAG |
| 481 | AGTTTAAGAG TCTTGTTTTT GGAGGAAAGC TCAATTGTTG |
| 521 | AGAAGGACGG AGAATGGTTA CACGAGCTTG CTTTGAATAA |
| 561 | TATAGTTCTT GAGACTCTCA ATTTTTACTT GACAGATATT |
| 601 | GCTGTTGTGA AGATTCAGGA CCTTGAACTT TTAGCTAAAA |
| 641 | ATTGCCCCAA CTTAGTGTCT GTGAAACTTA CTGACAGTGA |
| 681 | AATACTGGAT CTTGTGAACT TCTTTAAGCA TGCCTCTGCA |
| 721 | CTGGAAGAGT TTTGTGGAGG CACCTACAAT GAAGAACCAG |
| 761 | AAAAATACTC TGCTATATCA TTACCAGCAA AGTTATGTCG |
| 801 | ATTGGGTTTA ATATATATTG GAAAGAATGA GTTGCCCATA |
| 841 | GTGTTCATGT TTGCAGCCGT ACTAAAAAAA TTGGATCTCC |
| 881 | TCTATGCAAT GCTAGACACG GAGGATCATT GCATGTTAAT |
| 921 | CCAAAAGTGT CCAAATCTGG AAGTCCTTGA GACAAGGAAT |
| 961 | GTAATTGGAG ACAGAGGGTT AGAGGTTCTT GGTCGTTGTT |
| 1001 | GTAAGAGGCT AAAAAGGCTT AGGATTGAAA GGGGTGATGA |
| 1041 | TGATCAAGGA ATGGAGGATG AAGAAGGTAC TGTGTCCCAT |
| 1081 | AGAGGGCTAA TAGCCTTGTC ACAGGGCTGT TCAGAGCTTG |
| 1121 | AATACATGGC TGTTTATGTG TCTGATATTA CAAATGCATC |
| 1161 | TCTGGAACAT ATCGGAACTC ACTTGAAGAA CCTCTGCGAT |
| 1201 | TTTCGCCTTG TGTTGCTTGA CCACGAAGAG AAAATAACTG |
| 1241 | ATTTGCCACT TGACAATGGG GTGAGGGCTC TACTGAGGGG |
| 1281 | CTGTAACAAG CTGAGGAGAT TTGCTCTATA TCTCAGGCGT |
| 1321 | GGCGCGTTGA CCGATGTAGG TCTTGGTTAC ATTGGACAGT |
| 1361 | ACAGTCCAAA TGTGAGATGG ATGCTGCTTG GTTATGTGGG |
| 1401 | GGAGTCTGAT GCAGGGCTTT TGGAATTCTC TAAAGGGTGT |
| 1441 | CCTAGTCTTC AGAAACTAGA AATGAGAGGG TGTTCATTTT |
| 1481 | TCAGTGAACG TGCACTTGCT GTGGCTGCAA CACAATTGAC |
| 1521 | TTCTCTTAGG TACTTGTGGG TGCAAGGGTA TGGTGTATCT |
| 1561 | CCATCTGGAC GTGATCTTTT GGCAATGGCT CGCCCCTTTT |
| 1601 | GGAACATTGA GTTAATTCCT TCTAGAAAGG TGGCTATGAA |
| 1641 | TACCAATTCA GATGAGACGG TAGTTGTTGA GCATCCTGCT |
| 1681 | CATATTCTTG CATATTATTC TCTTGCAGGG CAGAGATCAG |
| 1721 | ATTTTCCAGA TACTGTTGTG CCTTTGGATA CTGCCACATG |
| 1761 | CGTTGACACC TAG |

A comparison of the Triticum aestivum (wheat) COI1 SEQ ID NO:1 sequence and the Glycine max (soybean) COI1 protein SEQ ID NO:21 sequence is shown below, illustrating that the two proteins have at least 60% sequence identity.

| 60.8% identity in 572 residues overlap; Score: 1793.0; Gap frequency: 0.9% | | |
|---|---|---|
| Seq1 | 22 | VPEEALHLVLGYVDDPRDREAASLACRRWHHIDALTRKHVTVPFCYAVSPARLLARFPRL |
| Seq21 | 12 | VVDLVLDCVIPYIDDPKDRDAVSQVCRRWYELDSLTRKHVTIALCYTTTPARLRRRFPHL |
| | | * * * * *** ** * * **** * * ******* ** **** *** * |
| Seq1 | 82 | ESLGVKGKPRAAMYGLIPDDWGAYARPWVAELAAPLECLKALHLRRMVVTDDDLAALVRA |
| Seq21 | 12 | ESLKLKGKPRAAMFNLIPEDWGGHVTPWVKEISQYFDCLKSLHFRRMIVKDSDLRNLARD |
| | | *** ******** *** *** *** * *** ** *** * * ** * * |
| Seq1 | 142 | RGHMLQELKLDKCSGFSTDALRLVARSCRSLRTLFLEECTITDNGTEWLHDLAANNPVLV |
| Seq21 | 132 | RGHVLHSLKLDKCSGFTTDGLFHIGRFCKSLRVLFLEESSIVEKDGEWLHELALNNTVLE |
| | | *** * ********* ** * * * *** ***** * **** ** ** ** |
| Seq1 | 202 | TLNFYLTYLRV-EPADLELLAKNCKSLISLKISDCDLSDLIGFFQIATSLQEFAGAEISE |
| Seq21 | 192 | TLNFYLTDIAVVKIQDLELLAKNCPNLVSVKLTDSEILDLVNFFKHASALEEFCGGTYNE |
| | | ******** * ********* * * * * ** ** * * ** * * |
| Seq1 | 261 | Q--KYGNVKLPSKLCSFGLTFMGTNEMHIIFPFSAVLKKLDLQYSFLTTEDHCQLIAKCP |
| Seq21 | 252 | EPEKYSAISLPAKLCRLGLTYIGKNELPIVFMFAAVLKKLDLLYAMLDTEDHCMLIQKCP |
| | | ** ** *** *** * ** * * * ******** * * ***** ** *** |
| Seq1 | 319 | NLLVLAVRNVIGDRGLGVVGDTCKKLQRLRVERGEDDPGMQEEEGGVSQVGLTAIAVGCR |
| Seq21 | 312 | NLEVLETRNVIGDRGLEVLGRCCKRLKRLRIERGDDDQGMEDEEGTVSHRGLIALSQGCS |
| | | ** ** ********* * * ** * *** *** ** ** *** ** ** * ** |
| Seq1 | 319 | ELENIAAYVSDITNGALESIGTFCKNLHDFRLVLLDKQETITDLPLDNGARALLRGCTKL |
| Seq21 | 312 | ELEYMAVYVSDITNASLEHIGTHLKNLCDFRLVLLDHEEKITDLPLDNGVRALLRGCNKL |
| | | *** * ******* ** *** *** ******** * ********* ******* ** |
| Seq1 | 439 | RRFALYLRPGGLSDVGLGYIGQHSGTIQYMLLGNVGQTDGGLISFAAGCRNLRKLELRSC |
| Seq21 | 432 | RRFALYLRRGGLTDVGLGYIGQYSPNVRWMLLGYVGESDAGLLEFSKGCPSLQKLEMRGC |
| | | ******** *** ********* * **** ** * *** ** * *** * * |
| Seq1 | 499 | CF-SERALALAIRQMPSLRYVWVQGYRASQTGRDLMLMARPFWNIEFTPPSTETAGRLME |
| Seq21 | 492 | SFFSERALAVAATQLTSLRYLWVQGYGVSPSGRDLLAMARPFWNIELIP-SRKVAMNTNS |
| | | * ****** * * **** ***** * **** ********* * * * |
| Seq1 | 558 | DGEPCVDRQAQVLAYYSLSGKRSDYPQSVVPL |
| Seq21 | 551 | DETVVVEHPAHILAYYSLAGQRSDFPDTVVPL |
| | | * * ****** * *** * **** |

An example of a COI1 protein from Zea mays (corn) with NCBI accession number NP_001150429.1 (GI:226503785) has the following sequence (SEQ ID NO:23).

| | |
|---|---|
| 1 | MGGEAPEPRR LTRALSIGGG DGGWVPEEML QLVMGFVEDP |
| 41 | RDREAASLVC HRWHRVDALS RKEVTVPFCY AVSPARLLAR |
| 81 | FPRLESLAVK GKPRAAMYGL IRDDWGAYAR PWITELAAPL |
| 121 | ECLKALHLRR MVVIDDDLAE LVRARCHMLQ ELKLDKCIGF |
| 161 | STHGLRLVAR SCRSLRTLFL EECQIDDKGS EWIHDLAVCC |
| 201 | PVLITLNEHM TELEVMPADL KLLAYSCKSL ISLKTSDCDL |
| 241 | SDLIEFFQFA TALEEFAGGT FNEQGELSKY VNVKFPSRLC |
| 281 | SLGLTYMGTN EMPIMFPFSA ILKKLDLQYT FLTTEDHCQL |
| 321 | IAKCPNLLVL AVRNVIGDRG LGVVADTCKK LQRLRIERGD |
| 361 | DEGGVQEEQG GVSQVGLTAI AVGCRELEYI AAYVSDITNG |
| 401 | ALESIGTFCK KLYDFRLVLL DREERITDLP LDNGVRALLR |
| 441 | GCTKLRRFAL YLRPGGLSDA GLGYIGQCSG NIQYMLLGNV |
| 481 | GETDDGLISF ALGCVNLRKL ELRSCCFSER ALALAILHMP |
| 521 | SLRYVWVQGY KASQTGRDLM LMARPFWNIE FTPPNPKNGG |
| 561 | WLMEDGEPCV DSHAQILAYH SLAGKRLDCP QSVVPLYPA |

An example of a nucleotide (cDNA) sequence that encodes the Zea mays SEQ ID NO:27 COI1 protein (with NCBI cDNA accession number NM_001156957.1, GI:226503784)) is shown below as SEQ ID NO:24.

| | |
|---|---|
| 1 | ACCCCTGCTT GCTGCAGCTT CAAGCACTAC CGAATCAGGG |
| 41 | CGAGTGGGAG CAGAGCAGGC AATCCCATGT CTCCGCCCCT |
| 81 | CGCTGGAGCA GATCGTTGTC GAGCCGACGT GGAGCTGCTG |
| 121 | CGGTAGAAAG CTAGCGGAGC CTGCGAGCTA GCCTGATCCG |
| 161 | TCCGCAGTCC GATCGGGATC GATCGGTGGG GAGGCGCCGG |
| 201 | AGCCGCGGCG GCTGATCCGG GCGCTGAGCA TCGGCGGCGG |
| 241 | TGACGGCGGC TGGGTTCCCG AGGAGATGCT GCAACTCGTG |
| 281 | ATGGGGTTCG TCGAGGACCC GCGCGATCGG GAGGCCGCGT |
| 321 | CGCTGGTGTG TCACCGGTGG CACCGCGTCG ACGCGCTCTC |
| 361 | GCGGAAGCAC GTGACGGTGC CCTTCTGCTA CGCCGTTTCC |
| 401 | CCGGCACGCC TGCTCGCGCG GTTCCCGCGG CTCGAGTCGC |
| 441 | TCGCGGTGAA GGGGAAGCCC CGCGCGGCCA TGTACGGGCT |
| 481 | CATACCCGAC GACTGGGGCG CCTACGCCCG CCCGTGGATC |
| 521 | ACCGAGCTCG CCGCGCCGCT CGAGTGCCTC AAGGCGCTCC |
| 561 | ACCTCCGACG CATGGTCGTC ACAGACGATG ATCTCGCCGA |
| 601 | GCTCGTCCGT GCCAGGGGGC ACATGCTGCA GGAGCTGAAG |
| 641 | CTCGATAAGT GCACCGGCTT CTCCACTCAT GGACTCCGCC |
| 681 | TCGTTGCCCG CTCCTGCAGA TCACTGAGGA CTTTATTTTT |
| 721 | GGAAGAATGT CAAATTGATG ATAAGGGCAG TGAATGGATC |
| 761 | CACGATCTCG CAGTCTGCTG TCCTGTTCTG ACAACATTGA |
| 801 | ATTTCCACAT GACTGAGCTT GAAGTGATGC CAGCTGATCT |
| 841 | AAAGCTTCTT GCAAAGAGCT GCAAGTCACT GATTTCATTG |
| 881 | AAGATTAGTG ACTGCGATCT TTCAGATTTG ATAGAGTTCT |
| 921 | TCCAATTTGC CACAGCACTG GAAGAATTTG CTGGAGGGAC |
| 961 | ATTCAATGAG CAAGGGGAAC TCAGCAAGTA TGTGAATGTT |
| 1001 | AAATTTCCAT CAAGACTATG CTCCTTGGGA CTTACTTACA |
| 1041 | TGGGAATAAA TGAAATGCCC ATTATGTTCC CTTTTTCTGC |
| 1081 | AATACTAAAG AAGCTGGATT TGCAATACAC TTTCCTCACC |
| 1121 | ACTGAGGACC ATTGCCAGCT CATTGCAAAA TGCCCGAACT |
| 1161 | TACTAGTTCT CGCGGTGAGG AATGTGATTG GAGATAGAGG |
| 1201 | ATTAGGAGTT GTTGCGGATA CGTGCAAGAA GCTCCAAAGG |
| 1241 | CTCAGAATAG AGCGAGGAGA TGATGAAGGA GGTGTGCAAG |
| 1281 | AAGAGCAGGG AGGGGTCTCT CAAGTGGGCT TGATGGCTAT |
| 1321 | AGCCGTAGGT TGCCGTGAGC TGGAATATAT AGCTGCCTAT |
| 1361 | GTGTCTGATA TAACCAATGG GGCCTTGGAA TCTATCGGGA |
| 1401 | CATTCTGCAA AAAACTATAC GACTTCCGGC TTGTTCTACT |
| 1441 | TGATAGAGAA GAGAGGATAA CAGACTTGCC ACTGGACAAT |
| 1481 | GGTGTCCGAG CTTTGTTGAG GGGCTGCACC AAGCTTCGGA |
| 1521 | GGTTTGCTCT GTATTTGAGA CCAGGAGGGC TCTCAGATGC |
| 1561 | AGGTCTCGGC TACATTGGAC AGTGCAGCGG AAACATCCAG |
| 1601 | TATATGCTTC TCGGTAATGT TGGGGAAATT GATGATGGAT |
| 1641 | TGATCAGCTT CGCATTGGGT TGCGTAAACC TGCGAAAGCT |
| 1681 | TGAACTCAGG AGTTGCTGCT TCAGCGAGCG AGCACTGGCC |
| 1721 | CTTGCAATAC TACATATGCC TTCCCTGAGG TACGTATGGG |
| 1761 | TTCAGGGCTA CAAAGCGTCT CAAACCGGCC GAGACCTCAT |
| 1801 | GCTCATGGCA AGGCCCTTCT GGAACATAGA GTTTACATCT |
| 1841 | CCCAATCCTA AGAACGGAGG TTGGCTGATG GAAGATGGGG |
| 1881 | AGCCTTGTGT AGATAGTCAC GCTCAGATAC TTGCATACCA |
| 1921 | CTCCCTCGCC GGTAAGAGGC TGGACTGCCC ACAATCCGTG |
| 1961 | GTTCCTTTGT ATCCTGCGTG AGTGTAAATA GACTAAGCTG |
| 2001 | GTGTCTTTCC TTAGCCTCCT GGTCAACAAG AATGGTGTTG |
| 2041 | ATAACTCGAT ATATGCGGTT ATTGTATGGA TCTAGATGGC |
| 2081 | TAGCTGCTAC GTATTGTAAT AAGCTACTAG TAGCTGAGAG |
| 2121 | ATGTCCTGGA ATAAGCCCTT GCTATTTTTG CCTAAAAAAA |
| 2161 | AAAAAAAAA |

A comparison of the Triticum aestivum (wheat) COI1 SEQ ID NO:1 sequence and the Zea mays (corn) COI1 protein SEQ ID NO:23 sequence is shown below, illustrating that the two proteins have at least 60% sequence identity.

| 83.8% itdentity in 599 residues overlap; Score: 2590.0; GAP frequency: 1.2% | | |
|---|---|---|
| Seq1 | 1 | MGGEAPEPRRLSRALSL---DGGGVPEEALHLVLGYVDDPRDREAASLACRRWHHIDALT |
| Seq23 | 1 | MGGEAPEPRRLTRALSIGGGDGGWVPEEMLQLVMGFVEDPRDREAASLVCHRWHRVDALS |
| | | **************** *** **** * ** * * ********** * *** *** |
| Seq1 | 58 | RKHVTVPFCYAVSPARLLARFPRLESLGVKGKPRAAMYGLIPDDWGAYARPWVAELAAPL |
| Seq23 | 61 | RKHVTVPFCYAVSPARLLARFPRLESLAVKGKPRAAMYGLIPDDWGAYARPWITELAAPL |
| | | *************************** ************************ ****** |
| Seq1 | 118 | ECLKALHLRRMVVTDDDLAALVRARGHMLQELKLDKCSGFSTDALRLVARSCRSLRTLFL |
| Seq23 | 121 | ECLKALHLRRMVVTDDDLAELVRARGHMLQELKLDKCTGFSTHGLRLVARSCRSLRTLFL |
| | | ******************* ***************** **** **************** |
| Seq1 | 178 | EECTITDNGTEWLHDLAANNPVLVTLNFYLTYLRVEPADLELLAKNCKSLISLKISDCDL |
| Seq23 | 181 | EECQIDDKGSEWIHDLAVDDPVLTTLNFHMTELEVMPADLKLLAKSCKSLISLKISDCDL |
| | | *** * * * ** **** *** **** * * * **** **** ************** |
| Seq1 | 238 | SDLIGFFQIATSLQEFAGAEISEQ----KYGNVKLPSKLCSFGLTFMGTNEMHIIFPFSA |
| Seq23 | 241 | SDLIEFFQFATALEEFAGGTFNEQGELSKYVNVKFPSRLCSLGLTYMGTNEMPIMFPFSA |
| | | **** *** ** * **** ** ** *** ** *** *** ****** ***** |
| Seq1 | 294 | VLKKLDLQYSFLTTEDHCQLIAKCPNLLVLAVRNVIGDRGLGVVGDTCKKLQRLRVERGE |
| Seq23 | 301 | ILKKLDLQYTFLTTEDHCQLIAKCPNLLVLAVRNVIGDRGLGVVADTCKKLQRLRIERGD |
| | | ******** ********************************** ********** *** |
| Seq1 | 354 | DDPGMQEEEGGVSQVGLTAIAVGCRELENIAAYVSDITNGALESIGTFCKNLHDFRLVLL |
| Seq23 | 361 | DEGGVQEEQGGVSQVGLTAIAVGCRELEYIAAYVSDITNGALESIGTFCKKLYDFRLVLL |
| | | * * *** ******************* ******************** * ******* |
| Seq1 | 414 | DKQETITDLPLDNGARALLRGCTKLRRFALYLRPGGLSDVGLGYIGQHSGTIQYMLLGNV |
| Seq23 | 421 | DREERITDLPLDNGVRALLRGCTKLRRFALYLRPGGLSDAGLGYIGQCSGNIQYMLLGNV |
| | | * * ********* ************************ ******* ** ********* |
| Seq1 | 474 | GQTDGGLISFAAGCRNLRKLELRSCCFSERALALAIRQMPSLRYVWVQGYRASQTGRDLM |
| Seq23 | 481 | GETDDGLISFALGCVNLRKLELRSCCFSERALALAILHMPSLRYVWVQGYKASQTGRDLM |
| | | * ** ****** ** ********************* ************ ********* |
| Seq1 | 534 | LMARPFWNIEFTPPSTETAGRLMEDGEPCVDRQAQVLAYYSLSGKRSDYPQSVVPLYPA |
| Seq23 | 541 | LMARPFWNIEFTPPNPKNGGWLMEDGEPCVDSHAQILAYHSLAGKRLDCPQSVVPLYPA |
| | | ************** * ********** ** *** ** *** * ********** |

An example of a COI1 protein from Arachis hypogaeal (peanut) with NCBI accession number AGH62009.1 (GI:469609864) has the following sequence (SEQ ID NO:25).

| | |
|---|---|
| 1 | RHCKKLQRLW IMDSIGDKGL GVVANTCKEL QELRVFPSDN |
| 41 | IGQHAAVTEK GLVAISMGCP KLHSLLYFCH QMTNAALITV |
| 81 | AKNCPNFIRF RLAILDATKP DPDTNQPLDE GFGAIVQSCR |
| 121 | RLRRLSLSGQ LTDKVFLYIG MYAEQLEMLS IAFAGESDKG |
| 161 | MLYVLNGCKK LRKLEIRDCP FGNTALLTDV GKYETMRSLW |
| 201 | MSSCEVTVGA CKVLAMKMPR LNVEIFNENE PADCEPDDVQ |
| 241 | KVEKMYLYRT LAGKRKDAPE YVWTL |

An example of a nucleotide (cDNA) sequence that encodes the Arachis hypogaeal (peanut) SEQ ID NO:38 COI1 protein (with NCBI cDNA accession number KC355791.1 (GI:469609863)) is shown below as SEQ ID NO:26.

| | |
|---|---|
| 1 | CGTCACTGCA AGAAACTTCA GCGCTTATGG ATAATGGATT |
| 41 | CCATTGGAGA TAAAGGGCTA GGTGTTGTAG CTAACACATG |
| 81 | TAAGGAATTG CAAGAATTGA GGGTTTTTCC TTCCGACAAC |
| 121 | ATTGGTCAGC ATGCGGCTGT CACAGAGAAG GGATTGGTTG |
| 161 | CGATATCTAT GGGCTGCCCG AAACTTCACT CATTGCTCTA |
| 201 | CTTCTGCCAC CAGATGACAA ATGCTGCCCT AATAACTGTG |
| 241 | GCCAAGAACT GCCCGAATTT TATCCGCTTT AGGTTGGCCA |
| 281 | TCCTTGACGC AACAAAACCC GACCCCGACA CAAATCAGCC |
| 321 | ACTGGATGAA GGTTTTGGGG CGATTGTGCA ATCTTGCAGG |
| 361 | CGTCTTAGGC GGCTTTCCCT CTCTGGCCAG CTGACTGATA |
| 401 | AGGTATTCCT CTACATCGGA ATGTATGCTG AGCAGCTTGA |
| 441 | GATGTTGTCC ATTGCCTTTG CCGGGGAGAG CGACAAGGGG |
| 481 | ATGCTCTATG TTCTGAACGG ATGCAAGAAG CTCCGCAAGC |
| 521 | TTGAGATCAG GGACTGCCCT TTCGGCAACA CGGCACTTCT |
| 561 | GACAGACGTA GGGAAGTATG AAACAATGCG ATCCCTTTGG |
| 601 | ATGTCGTCGT GCGAAGTAAT CGTCGGAGCA TGCAAGGTGC |
| 641 | TAGCAATGAA GATGCCGAGG CTAAATGTTG AGATCTTCAA |
| 681 | CGAGAATGAG CCAGCCGACT GCGAGCCGCA TGATGTGCAG |
| 721 | AAGGTGCAGA AGATGTACTT GTACCGGACA TTGGCTGGGA |
| 761 | AGAGGAAAGA TGCACCGGAA TATGTATGGA CCCTTTAGGT |
| 801 | GCATTTTTAG GTCAATTTTA ATTTTATTGT TATTATTGAG |
| 841 | CAGTTTGTAC GTTAGGCTGA CTTATTAATG CAATTTTAGC |
| 881 | CTTGTGTAGT GGTTGGTTTG |

A comparison of the Triticum aestivum (wheat) COI1 SEQ ID NO:11 sequence and the Arachis hypogaeal (peanut) COI1 protein SEQ ID NO:25 sequence is shown below, illustrating that the two proteins have at least 33% sequence identity.

| 33.5% identity in 272 residues overlap; Score: 314.0; Gap frequency: 5.1% | | |
|---|---|---|
| Seq1 | 317 | CPNLLVLAVRNVIGDRGLGVVGDTCKKLQRLRVERGEDDPGMQEEEGGVSQVGLTAIAVG |
| Seq23 | 3 | CKKLQRLWIMDSIGDKGLGVVANTCKELQELRVFPS-DNIG---QHAAVTEKGLVAISMG |
| | | * * * *** ***** *** ** *** * * * ** ** * |
| Seq1 | 311 | CRELENIAAYVSDITNGALESIGTFCKNLHDFRLVLLD--KQETITDLPLDNGARALLRG |
| Seq23 | 59 | CPKLHSLLYFCHQMTNAALITVAKNCPNFIRFRLAILDATKPDPETNQPLDEGFGAIVQS |
| | | * * ** ** * * *** ** * * *** * * |
| Seq1 | 435 | CTKLRRFALYLRPGGLSDVGLGYIGQHSGTIQYMLLGNVGQTDGGLISFAAGCRNLRKLE |
| Seq23 | 119 | CRRLRRLSL---SGQLTDKVFLYIGMYAEQLEMLSIAFAGESDKGMLYVLNGCKKLRKLE |
| | | * *** * * * * * *** * * ** ***** |
| Seq1 | 495 | LRSCCFSERALALAIRQMPSLRYVWVQGYRASQTGRDLMLMARPFWNIEFTPPSTETAGR |
| Seq23 | 116 | IRDCPFGNTALLTDVGKYETMRSLWMSSCEVTVGACKVLAMKMPRLNVEIFN-ENEPADC |
| | | * * * ** * * * * * * * * |
| Seq1 | 555 | LMEDGEPCVDRQAQVLAYYSLSGKRSDYPQSV |
| Seq23 | 235 | EPDD----VQKVEKMYLYRTLAGKRKDAPEYV |
| | | * * * * *** * * * |

In some cases, the COI1 protein can have a sequence related to SEQ ID NO:1, 2, 5, 8, 10, 13, 16, 19, 22, 25, 28, 31, 33, 36, 39, 42, 45, or 48. However, the modified COI1 protein can have some sequence variation relative to SEQ ID NO:1, 2, 5, 8, 10, 13, 16, 19, 22, 25, 28, 31, 33, 36, 39, 42, 45, or 48. For example, a modified COI1 protein can have an amino acid sequence that has at least 90%, or at least 95%, or at least 96%, or at least 97%, at least 98%, or at least 99% sequence identity to SEQ ID NO:1, 2, 5, 8, 10, 13, 16, 19, 22, 25, 28, 31, 33, 36, 39, 42, 45, or 48.

### Thaumatin-Like Protein (Tlp1)

The Tlp1 protein expressed by the expression cassette, plant cells, plants, and plant seeds can have a variety of sequences. An example of a Tlp1 protein from Triticum aestivum (wheat) with NCBI accession number CAA41283.1 has the following sequence (SEQ ID NO:27).

| | |
|---|---|
| 1 | MATSPVLFLL LAVFAAGASA ATFNIKNNCG FTIWPAGIPV |
| 41 | GGGFALGSGQ TSSINVPAGT QAGRIWARTG CSFNGGSGSC |
| 81 | QTGDCGGQLS CSLSGRPPAT LAEYTIGGGS TQDFYDISVI |
| 121 | DGFNLAMDFS CSTGDALQCR DPSCPPPQAY QHPNDVATHA |
| 161 | CSGNNNYQIT FCP |

An example of a nucleotide (cDNA) sequence that encodes the Triticum aestivum (wheat) SEQ ID NO:27 Tlp1 protein (with NCBI cDNA accession number X58394.1) is shown below as SEQ ID NO:28.

| | |
|---|---|
| 1 | CCTACAGCAA AGCTCGAGCA TAGCAACAGC ACTAAAGCTA |
| 41 | ACTAGAGCTT CCAGCAATGG CGACCTCCCC GGTGCTCTTC |
| 81 | CTCCTCCTCG CTGTTTTCGC CGCCGGTGCC ACCGCGGCCA |
| 121 | CCTTCAACAT CAAGAACAAC TGTGGCTTCA CAATTTGGCC |
| 161 | GGCGGGCATC CCGGTGGGTG GGGGCTTCGC GCTGGGCTCA |
| 201 | GGGCAGACGT CCAGCATCAA CGTGCCCGCG GGCACCCAAG |
| 241 | CCGGGAGGAT ATGGGCCCGC ACCGGGTGCT CCTTCAATGG |
| 281 | CGGTAGCGGG AGCTGCCAGA CCGGCGACTG CGGCGGCCAG |
| 321 | CTATCCTGCT CCCTCTCCGG GCGGCCACCA GCAACGCTGG |
| 361 | CCGAGTACAC CATCGGCGGC GGCAGCACCC AGGACTTCTA |
| 401 | CGACATCTCG GTGATCGACG GCTTCAACCT TGCCATGGAC |
| 441 | TTCTCGTGCA GCACCGGCGA CGCGCTCCAG TGCAGGGACC |
| 481 | CCAGCTGCCC GCCGCCGCAA GCCTACCAAC ACCCGAACGA |
| 521 | CGTCGCCACA CACGCCTGCA GTGGCAATAA TAACTACCAG |
| 561 | ATCACCTTCT GTCCATGAAG CCTCTATACG TCGCACCGCG |
| 601 | AATCAATAAA AGGCGTACGT AGATATACGG CCATATAAAT |
| 641 | AAAAGGTGTA CTGCTTAATA AAAAAAAAAA AAAA |

A second example of a Tlp1 protein from Triticum aestivum (wheat) with NCBI accession number AAK60568.1 has the following sequence (SEQ ID NO:29).

| | |
|---|---|
| 1 | MATSAVLFLL LAVFAAGASA ATFNIKNNCG STIWPAGIPV |
| 41 | GGGFELGAGQ TSSINVPAGT KAGRIWARTG CSFNGGSGSC |
| 81 | RTGDCGGQLS CSLSGRPPAT LAEYTIGGGG TQDFYDISVI |
| 121 | DGFNLAMDFS CSTGDALQCR DPSCPPPQAY QHPNDQATHA |
| 161 | CSGNNNYQIT FCP |

An example of a nucleotide (cDNA) sequence that encodes the Triticum aestivum (wheat) SEQ ID NO:29 Tlp1 protein (with NCBI cDNA accession number AF384146.1) is shown below as SEQ ID NO:30.

| | |
|---|---|
| 1 | CCACGCGTCC GATGGCGACC TCCGCGGTGC TCTTCCTCCT |
| 41 | CCTCGCTGTT TTTGCCGCCG GTGCCAGCGC GGCCACCTTC |
| 61 | AACATCAAGA ACAACTGCGG CTCCACAATT TGGCCGGCGG |
| 121 | GCATCCCGGT GGGTGGGGGC TTCGAGCTGG GCGCAGGCCA |
| 161 | GACGTCCAGC ATCAATGTGC CCGCGGGCAC CAAAGCCGGG |
| 201 | AGGATATGGG CTCGCACCGG GTGCTCCTTC AATGGCGGCA |
| 241 | GCGGGAGCTG CCGGACCGGT GACTGCGGCG GCCAGCTGTC |
| 281 | CTGCTCCCTC TCCGGGCGGC CACCAGCAAC GCTGGCCGAG |
| 321 | TATACCATCG GCGGCGGCGG CACCCAGGAC TTCTATGACA |
| 361 | TCTCGGTGAT CGATGGCTTC AACCTTGCCA TGGACTTCTC |
| 401 | GTGCAGTACC GGCGACGTGC TCCAGTGCAG GGATCCCAGT |
| 441 | TGCCCGCCGC CGCAAGCCTA CCAACACCCC AACGACCAAG |
| 481 | CCACACACGC CTGCAGTGGC AATAATAACT ACCAGATCAC |
| 521 | CTTCTGCCCA TGAAGGCTGT TCACGTCGCA CCATGAATCA |
| 561 | ATAAAAGGTG TACGTAGATA TACGGCCCTA TAAATAAAAG |
| 601 | GCGTGCTGCT TAAAAAAAAA AAAAAAAAAA AAAAAAAAAA |
| 641 | AAAAAAAAAA AAAAAAAAAA AAA |

A comparison of the Triticum aestivum (wheat) Tlp1 SEQ ID NO:27 sequence and the Triticum aestivum (wheat) Tlp1 protein SEQ ID NO:29 sequence is shown below, illustrating that the two proteins have at least 95% sequence identity.

| 95.4% identity in 173 residues overlap; Score: 904.0; Gap frequency: 0.0% | | |
|---|---|---|
| Seq27 | 1 | MATSPVLFLLLAVFAAGASAATFNIKNNCGFTIWPAGIPVGGGFALGSGQTSSINVPAGT |
| Seq29 | 1 | MATSAVLFLLLAVFAAGASAATFNIKNNCGSTIWPAGIPVGGGFELGAGQTSSINVPAGT |
| | | **** ************************* ************* ** ************ |
| Seq27 | 61 | QAGRIWARTGCSFNGGSGSCQTGDCGGQLSCSLSGRPPATLAEYTIGGGSTQDFYDISVI |
| Seq29 | 61 | KAGRIWARTGCSFNGGSGSCRTGDCGGQLSCSLSGRPPATLAEYTIGGGGTQDFYDISVI |
| | | ******************* **************************** *********** |
| Seq21 | 121 | DGFNLAMDFSCSTGDALQCRDPSCPPPQAYQHPNDVATHACSGNNNYQITFCP |
| Seq29 | 121 | DGFNLAMDFSCSTGDALQCRDPSCPPPQAYQHPNDQATHACSGNNNYQITFCP |
| | | *********************************** ***************** |

An example of a Hordeum vulgare (domesticated barley) Tlp1 protein with a sequence provided by the NCBI database as accession number P32938.1, is shown below as SEQ ID NO:31.

| | |
|---|---|
| 1 | MSTSAVLFLL LAVFAAGASA ATFNIKNNCG STIWPAGIPV |
| 41 | GGGFELGSGQ TSSINVPAGT QAGRIWARTG CSFNGGSGSC |
| 81 | QTGDCGGQLS CSLSGRPPAT LAEFTIGGGG TQDFYDISVI |
| 121 | DGFNLAMDFS CSTGDALQCR DPSCPPPQAY QHPNDVATHA |
| 161 | CSGNNNYQIT FCP |

A comparison of the Triticum aestivum (wheat) Tlp1 SEQ ID NO:27 sequence and the Hordeum vulgare (domesticated barley) Tlp1 protein SEQ ID NO:31 sequence is shown below, illustrating that the two proteins have at least 97% sequence identity.

| 97.1% identity in 173 residues overlap; Score: 918.0; Gap frequency: 0.0% | | |
|---|---|---|
| Seq27 | 1 | MATSPVLEILLAVFAAGASAATFNIKNNCGFTIWPAGIPVGGGFALGSGQTSSINVPAGT |
| Seq29 | 1 | MSTSAVLEILLAVFAAGASAATFNIKNNCGSTIWPAGIPVGGGFELGSGQTSSINVPAGT |
| | | * ** ************************* ************* *************** |
| Seq27 | 61 | QAGRIWARTGCSFNGGSGSCQTGDCGGQLSCSLSGRPPATLAEYTIGGGSTQDFYDISVI |
| Seq29 | 61 | QAGRIWARTGCSFNGGSGSCQTGDCGGQLSCSLSGRPPATLAEFTIGGGSTQDFYDISVI |
| | | ******************************************* **************** |
| Seq27 | 121 | DGFNLAMDFSCSTGDALQCRDPSCPPPQAYQHPNDVATHACSGNNNYQITFCP |
| Seq29 | 121 | DGFNLAMDFSCSTGDALQCRDPSCPPPQAYQHPNDVATHACSGNNNYQITFCP |
| | | ***************************************************** |

An example of a Triticum urartu (red wild einkorn) Tip 1 protein with a sequence provided by the NCBI database as accession number EMS68875.1, is shown below as SEQ ID NO:32.

| | |
|---|---|
| 1 | MATSAVLFLL LAVFAAGASA ATFNIKNNCG STIWPAGIPV |
| 41 | GGGFALGAGQ TSSINVPAGT KAGRIWAPTG CSFNGGSGSC |
| 81 | RTGDCGGQLS CSLSGRPPAT LAEYTIGGGS TQDFYDISVI |
| 121 | DGFNLAMDFS CSTGDALQCR DPSCPPPQAY QHPNDQATHA |
| 161 | CSGNNNYQIT FCP |

A comparison of the Triticum aestivum (wheat) Tlp1 SEQ ID NO:27 sequence and the Triticum urartu (red wild einkorn) Tlp1 protein SEQ ID NO:32 sequence is shown below, illustrating that the two proteins have at least 96% sequence identity.

| 96.5% identity in 173 residues overlap; Score: 913.0; Gap frequency: 0.0% | | |
|---|---|---|
| Seq27 | 1 | MATSPVLFLLLAVFAAGASAATFNIKNNCGFTIWPAGIPVGGGFALGSGQTSSINVPAGT |
| Seq30 | 1 | MATSATLFLLLAVFAAGASAATFNIKNNCGSTIWPAGIPVGGGFALGAGQTSSINVPAGT |
| | | **** ************************* **************** ************ |
| Seq27 | 61 | QAGRIWARTGCSFNGGSGSCQTGDCGGQLSCSLSGRPPATLAEYTIGGGSTQDFYDISVI |
| Seq30 | 61 | KAGRIWARTGCSFNGGSGSCRTGDCGGQLSCSLSGRPPATLAEYTIGGGSTQDFYDISVI |
| | | ******************* **************************************** |
| Seq27 | 121 | DGFNLAMDFSCSTGDALQCRDPSCPPPQAYQHPNDVATHACSGNNNYQITFCP |
| Seq30 | 121 | DGFNLAMDFSCSTGDALQCRDPSCPPPQAYQHPNDQATHACSGNNNYQITFCP |
| | | *********************************** ***************** |

An example of an Aegilops tauschii (goat grass) Tlp1 protein with a sequence provided by the NCBI database as accession number EMT13094.1, is shown below as SEQ ID NO:33.

| | |
|---|---|
| 1 | MATSPVLFLL LAVFAAGASA ATFNIKNNCG STIWPAGIPV |
| 41 | GGGFALGAGQ TSSINVPAGT KAGRIWARTG CSFNGGSGSC |
| 81 | QTGDCGGQLS CSLSGRPPAT LAEYTIGGGS TQDFYDISVI |
| 121 | DGFNLAMDFS CSTGDALQCR DPSCPPPQAY QHPDDRATHA |
| 161 | CNGNSNYQIT FCP |

A comparison of the Triticum aestivum (wheat) Tlp1 SEQ ID NO:27 sequence and the Aegilops tauschii (goat grass) Tlp1 protein SEQ ID NO:33 sequence is shown below, illustrating that the two proteins have at least 96% sequence identity.

| 96.0% identity in 113 residues overlap; Score: 911.0; Gap frequency: 0.0% | | |
|---|---|---|
| Seq27 | 1 | MATSPVLFLLLAVFAAGASAATFNIKNNCGFTIWPAGIPVGGGFAIGSGQTSSINVPAGT |
| Seq31 | 1 | MATSPVLFLLLAVFAAGASAATFNIKNNCGSTIWPAGIPVGGGFAIGAGQTSSINVPAGT |
| | | ****************************** ***************************** |
| Seq27 | 61 | QAGRIWARTGCSFNGGSGSCQTGDCGGQLSCSLSGRPPATLAEYTIGGGSTQDFYDISVI |
| Seq31 | 61 | KAGRIWARTGCSFNGGSGSCQTGDCGGQLSCSLSGRPPATLAEYTIGGGSTQDFYDISVI |
| | | *********************************************************** |
| Seq27 | 121 | DGFNLAMDFSCSTGDALQCRDPSCPPPQAYQHPNDVATHACSGNNNYQITFCP |
| Seq31 | 121 | DGFNLAMDFSCSTGDALQCRDPSCPPPQAYQHPDDRATHACNGNSNYQITFCP |
| | | ********************************* * ***** ** ******** |

An example of a Secale cereale (rye) Tlp1 protein with a sequence provided by the NCBI database as accession number AAC67259.1, is shown below as SEQ ID NO:34.

| | |
|---|---|
| 1 | MATSAVLFLL FAVFAAGASA ATFNIKNNCG STIWPAGIPV |
| 41 | GGAFALGSGQ TSSINVPAGT QAGRIWARTG CSFNGGTGSC |
| 81 | QTGDCGGQLS CSLSGRPPAT LAEFTIGGGS TQDFYDISVI |
| 121 | DGFNLAMDFS CSTGDALQCR DPSCPPPQAY QHPNDMATHA |
| 161 | CRGNSNYQIT FCP |

An example of a nucleotide (cDNA) sequence that encodes the Secale cereale (rye) SEQ ID NO:34 Tlp1 protein (with NCBI cDNA accession number AF096927.1) is shown below as SEQ ID NO:35.

| | |
|---|---|
| 1 | GGCACGAGGC TAACTAGAGC TTGCAGCAAT GGCGACCTCT |
| 41 | GCGGTGCTCT TCCTCCTCTT CGCTGTTTTT GCCGCCGGTG |
| 81 | CCAGCGCGGC CACCTTCAAC ATCAAGAACA ATTGCGGCTC |
| 121 | CACAATTTGG CCGGCGGGCA TCCCGGTGGG TGGGGCGTTC |
| 161 | GCGCTGGGCT CAGGCCAGAC GTCCAGCATC AACGTACCCG |
| 201 | CAGGAACCCA AGCCGGGAGG ATATGGGCCC GCATCGGGTG |
| 241 | CTCCTTCAAT GGCGGTACGG GGAGCTGCCA GACCGGCGAC |
| 281 | TGCGGTGGCC AGCTGTCCTG CTCCCTCTCC GGGCGGCCAC |
| 321 | CAGCAACGCT TGCCGAGTTC ACCATCGGCG GCGGCAGCAC |
| 361 | CCAGGATTTC TACGACATCT CGGTGATCGA CGGCTTCAAT |
| 401 | CTTGCCATGG ACTTCTCATG CAGCACCGGC GACGCGCTAC |
| 441 | AGTGCAGGGA TCCCAGCTGC CCACCGCCGC AAGCCTACCA |
| 481 | ACACCCCAAC GACATGGCCA CACACGCCTG CAGAGGCAAT |
| 521 | AGTAACTATC AGATCACCTT CTGCCCATGA AGCATGTTTA |
| 561 | CGTCGCACCT CCCATCTATA AAGGCGTACG TAGATATATG |
| 601 | GCCGTATAAA TTAAAGGTGT GCTGCTTAAT ACTCCCTCTG |
| 641 | TAAACTAATA TAAAAGCATT TAGATCACTA AAGTAGTGTT |
| 681 | CTAAACACTC TTATATTAGT TTACGGAGGG AGTACATCAC |
| 721 | AGATCACACT GTCATATTAC AGCCACTGTA CTACTATATT |
| 761 | GAATGGCAGA GGAGCCGATT CCGGGCAGAG AAGCCGAAGC |
| 801 | AGGGGAGCCA GAGAGAGAGA GAGAGTTGAA GGAAGAGAGG |
| 841 | ATATATTTTC GCTCACTCTA CTCACTACTG TGAGGGTTTT |
| 881 | ATTGATACTA GTAAGCTTGT ACGTGCAAAT GCACGTCTCG |
| 921 | ACTAATATTA CAAACGAAGG TCTACGCGTC GGCCGGCGGA |
| 961 | TCTTTTTAAA GATCCGCCGC CTCACGAGCC GTCCGATATA |
| 1001 | AGTTGCTTAA CATGCTATCC CACTTCGCAA CAATAGTGTT |
| 1041 | GTTTCAGAAA CAATGGTGAC TATTCCAACA AAGAGCTTTA |
| 1081 | TTTCAGAAAA AAAAAAAAAA AAAAAAAAAA AAAAAAAAAA |
| 1121 | AAAAAAAAAA AAAAAAAAAA AAAAAAAAAA A |

A comparison of the Triticum aestivum (wheat) Tlp1 SEQ ID NO:27 sequence and the Secale cereale (rye) Tlp1 protein SEQ ID NO:34 sequence is shown below, illustrating that the two proteins have at least 94% sequence identity.

| 94.8% identity in 173 residues overlap; Score: 900.0; Gap frequency: 0.0% | | |
|---|---|---|
| Seq27 | 1 | MATSPVLEILLAVFAAGASAATFNIKNNCGFTIWPAGIPVGGGFALGSGQTSSINVPAGT |
| Seq32 | 1 | MATSAVLEILFAVFAAGASAATFNIKNNCGSTIWPAGIPVGGAFALGSGQTSSINVPAGT |
| | | **** ***** ******************* ***************************** |
| Seq27 | 61 | QAGRIWARTGCSYNGGSGSCQTGDCGGQLSCSLSGRPPATLAEYTIGGGSTQDFYDISVI |
| Seq32 | 61 | QAGRIWARTGCSFNGGTGSCQTGDCGGQLSCSLSGRPPATLAEFTIGGGSTQDFYDISVI |
| | | **************** ************************** **************** |
| Seq27 | 121 | DGFNLAMDFSCSTGDALQCRDPSCPPPQAYQHPNDVATHACSGNNNYQITFCP |
| Seq32 | 121 | DGFNLAMDFSCSTGDALQCRDPSCPPPQAYQHPNDMATHACRGNSNYQITFCP |
| | | *********************************** ***** ** ******** |

An example of an Avena sativa (oat) Tlp1 protein with a sequence provided by the NCBI database as accession number P50695.1, is shown below as SEQ ID NO:36.

| | |
|---|---|
| 1 | MATSSAVLFL LLAVFAAGAS AATFRITNNC GFTVWPAGIP |
| 41 | VGGGFQLNSK QSSNINVPAG TSAGRIWGRT GCSFNNGRGS |
| 81 | CATGDCAGAL SCTLSGQPAT LAEYTIGGSQ DFYDISVIDG |
| 121 | FNLAMDFSCS TGVALKCRDA NCPDAYHHPN DVATHACNGN |
| 141 | SNYQITFCP |

A comparison of the Triticum aestivum (wheat) Tlp1 SEQ ID NO:27 sequence and the Avena sativa (oat) Tlp1 protein SEQ ID NO:36 sequence is shown below, illustrating that the two proteins have at least 80% sequence identity.

| 80.7% identity in 171 residues overlap; Score: 708.0; Gap frequency: 2.9% | | |
|---|---|---|
| Seq27 | 3 | TSPVLFLLLAVFAAGASAATFNIKNNCGFTIWPAGIPVGGGFALGSGDTSSINVPAGTQA |
| Seq34 | 4 | SSAVLFLLLAVFAAGASAATFRITNNCGFTVWPAGIPVGGGFQLNSKDSSNINVPAGTSA |
| | | * ****************** * ****** *********** * * * * ******* * |
| Seq27 | 63 | GRIWARTGCSFNGGSGSCQTGDCGGQLSCSLSGRPPATLAEYTIGGGSTQDFYDISVIDG |
| Seq34 | 64 | GRIWGRTGCSFNNGRGSCATGDCAGALSCTLSGQP-ATLAEYTIGG--SQDFYDISVIDG |
| | | **** ******* * *** **** * *** *** * ********** *********** |
| Seq27 | 123 | FNLAMDFSCSTGDALQCRDPSCPPPQAYQHPNDVATHACSGNNNYQITFCP |
| Seq34 | 121 | FNLAMDFSCSTGVALKCRDANCP--DAYHHPNDVATHACNGNSNYQITFCP |
| | | ************ ** *** ** ** ********** ** ******** |

A third example of a Tlp1 protein from Triticum aestivum (wheat) with NCBI accession number AIG62904.1 has the following sequence (SEQ ID NO:37).

| | |
|---|---|
| 1 | MATSAVLFLL LAVFAAGASA ATFYIKNNCG STIWPAGIPV |
| 41 | GGGFALGSGQ TASINVPAGT KAGRIWARTG CSFNGGSGSC |
| 81 | QTGDCGGQLS CSLSGRPPAT LTENTIGGAQ DFYDISVIDG |
| 721 | FNLAMDFSCG TGDALQCRDP SCPPPQAYQH PNDVATHACN |
| 161 | GNSNYQITFC P |

An example of a nucleotide (cDNA) sequence that encodes the Triticum aestivum (wheat) SEQ ID NO:37 Tlp1 protein (with NCBI cDNA accession number KJ764822.1) is shown below as SEQ ID NO:38.

| | |
|---|---|
| 1 | ATGGCGACCT CCGCGGTGCT CTTCCTCCTC CTGGCTGTTT |
| 41 | TCGCCGCCGG TGCCAGCGCG GCCACCTTCT ACATCAAGAA |
| 81 | CAACTGCGGC TCCACAATTT GGCCGGCGGG CATCCCGGTG |
| 121 | GGTGGGGGCT TCGCGCTGGG CTCAGGCCAG ATGGCCAGCA |
| 161 | TCAACGTGCC CGCGGGCACC AAAGCCGGGA GGATATGGGC |
| 201 | CCGCACCGGG TGCTCCTTCA ATGGTGGTAG CGGGAGCTGC |
| 241 | CAGACCGGCG ACTGCGGAGG CCAGCTGTCC TGCTCCCTCT |
| 281 | CCGGGCGGCC ATCGGCAACG CTGACCGAGA ACACCATCGG |
| 321 | CGGCGCCCAA GACTTCTACG ACATCTCGGT GATCGACGGC |
| 361 | TTCAACCTTG CCATGGACTT CTCGTGTGGC ACCGGCGACG |
| 401 | CGCTCCAGTG CAGGGACCCC AGCTGCCCGC CGCCGCAAGC |
| 441 | CTACCAACAC CCCAACGACG TCGCCACATA CTCTTTCAAT |
| 481 | GGCAACAGTA ACTACCAGAT CACCTTCTGT CCATGA |

A comparison of the Triticum aestivum (wheat) Tlp1 SEQ ID NO:27 sequence and the third Triticum aestivum (wheat) Tlp1 protein SEQ ID NO:37 sequence is shown below, illustrating that the two proteins have at least 92% sequence identity.

| 92.5% identity in 173 residues overlap; Score: 855.0; Gap frequency: 1.2% | | |
|---|---|---|
| Seq27 | 1 | MATSPVLFLLLAVFAAGASAATFNIKNNCGFTIWPAGIPVGGGFALGSGQTSSINVPAGT |
| Seq35 | 1 | MATSAVLFLLLAVFAAGASAATFYIKNNCGSTIWPAGIPVGGGFALGSGQTASINVPAGT |
| | | **** ****************** ****** ******************** ******** |
| Seq27 | 61 | QAGRIWARTGCSFNGGSGSCQTGDCGGQLSCSLSGRPPATLAEYTIGGGSTQDFYDISVI |
| Seq35 | 61 | KAGRIWARTGCSFNGGSGSCQTGDCGGQLSCSLSGRPPATLTENTIGGA--QDFYDISVI |
| | | **************************************** * **** ********* |
| Seq27 | 121 | DGFNLAMDFSCSTGDALQCRDPSCPPPQAYQHPNDVATHACSGNNNYQITFCP |
| Seq35 | 119 | DGFNLAMDFSCGTGDALQCRDPSCPPPQAYQHPNDVATHACNGNSNYQITFCP |
| | | *********** ***************************** ** ******** |

An example of a Tlp1 protein from Zea mays (maize) with NCBI accession number NP_001141293.1 has the following sequence (SEQ ID NO:39).

| | |
|---|---|
| 1 | MTSSSVFFLL LACFATCAGA ATFTVRNNCG FTVWPAGIPV |
| 41 | GGGTQLNPGS TWTVNVQAGT SGGRIWGRTG CSFSGGRGRC |
| 81 | ATGDCGGAYS CSLSGQPPAT LAEFTIGGGS NHDYYDISVI |
| 721 | DGYNLPMDFS CSTGAALRCR DSGCPDAYHQ PNDPKTRSCN |
| 161 | GNSNYQVVFC P |

An example of a nucleotide (cDNA) sequence that encodes the Zea mays (maize) SEQ ID NO:39 Tlp1 protein (with NCBI cDNA accession number NM_001147821.2) is shown below as SEQ ID NO:40.

| | |
|---|---|
| 1 | ACTATATATG TATATAGACG TGGTCAGGAT GACGTCGTCC |
| 41 | TCCGTCTTCT TCCTCCTGCT CGCTTGCTTC GCCACATGCG |
| 81 | CCGGCGCCGC CACGTTCACG GTACGAAACA ACTGCGGGTT |
| 121 | CATGGTGTGG CCAGCAGGCA TCCCGGTCGG CGGCGGCACG |
| 161 | CAGCTGAACC CGGGCTCGAC GTGGACTGTC AACGTGCAGG |
| 201 | CCGGGACCAG CGGAGGCAGG ATCTGGGGTC GCACCGGCTG |
| 241 | CTCCTTCAGC GGCGGCCGGG GCCGCTGCGC GACGGGCGAC |
| 281 | TGCGGAGGCG CCTACTCCTG CAGCCTGTCC GGGCAGCCCC |
| 321 | CGGCGACGCT GGCCGAGTTC ACCATCGGCG GCGGCAGCAA |
| 361 | CCACGACTAC TACGACATCT CGGTGATCGA CGGGTACAAC |
| 401 | CTGCCCATGG ACTTCTCGTG CAGCACCGGC GCCGCCCTCC |
| 441 | GGTGCAGGGA CTCCGGCTGC CCCGACGCGT ATCACCAGCC |
| 481 | CAACGATCCT AAGACCCGTT CGTGCAACGG CAATAGCAAT |
| 521 | TACCAGGTCG TCTTCTGTCC TTGATGTACA TGCATGCAGT |
| 561 | TTGCCATGAG CCCATGCATG CATGAGAGCG TAGCGGCATA |
| 601 | TATACATTAA TTGTCCGTCA ACTCACATGC ATGTATCTAT |
| 641 | TAGTGAAGTT GATAAATAAG ACGATCGATA TCTGATTCGT |
| 681 | CCAATGCAGC ATGTGTGTAC GGTGTGATCC TTATACTTTC |
| 721 | CTACATATAT GGAACTTTTT AAAGTGAGTA AAGTGC |

A comparison of the Triticum aestivum (wheat) Tlp1 SEQ ID NO:27 sequence and the Zea mays (maize) Tlp1 protein SEQ ID NO:39 sequence is shown below, illustrating that the two proteins have at least 68% sequence identity.

| 68.8% identity in 113 residues overLap; Score: 659.0; Gap frequency: 1.2% | | |
|---|---|---|
| Seq27 | 1 | MATSPVLEILLAVFAAGASAATFNIKNNCGFTIWPAGIPVGGGFALGSGQTSSINVPAGT |
| Seq37 | 1 | MTSSSVFEILLACFATCAGAATFTVRNNCGFTVWPAGIPVGGGTQLNTGSTWTVNVQAGT |
| | | * * * ***** ** * **** ****** ********** * * * ** *** |
| Seq27 | 61 | QAGRIWARTGCSFNGGSGSCQTGDCGSQLSCSLSGRPPATLAEYTIGGGSTQDFYDISVI |
| Seq37 | 61 | SGGRIWGRTGCSFSGGRGRCATGDCGGAYSCSLSGQPPATLAEFTIGGGSNHDYYDISVI |
| | | **** ****** ** * * ***** ****** ******* ****** * ****** |
| Seq21 | 121 | DGFNLAMDFSCSTGDALQCRDPSCPPPQAYQHPNDVATHACSGNNNYQITFCP |
| Seq31 | 121 | DGYNLPMDFSCSTGAALRCRDSGCP--DAYHQPNDPKTRSCNGNSNYQVVFCP |
| | | ** ** ******** ** *** ** ** *** * * ** *** *** |

An example of a Tip 1 protein from Sorghum bicolor (sorghum) with NCBI accession number XP_002443621.1 has the following sequence (SEQ ID NO:41).

| | |
|---|---|
| 1 | MAASSSSILL LFLAATLMAS GTHAATFTIK NNCGFTVWPA |
| 41 | ATPVGGGTQL NSGGTWTINV PAGTSSGRVW GRTGCSFNGN |
| 81 | SGSCQTGDCG GALACTLSGK PPLTLAEFTI GGSQDFYDIS |
| 121 | VIDGFNIGMA FSCSTGVGLV CRDSSCPDAY HNPPDRKTHA |
| 161 | CGGNSNYQVT FCP |

An example of a nucleotide (cDNA) sequence that encodes the Sorghum bicolor (sorghum) SEQ ID NO:41 Tlp1 protein (with NCBI cDNA accession number XM_002443576.1) is shown below as SEQ ID NO:42.

| | |
|---|---|
| 1 | AAGCAGCTCG CCTATTTCCA CAAGAAAGAA GCTCGCATAG |
| 41 | TATTAGCATC AATATATGGC CGCCTCATCG TCCTCGATCC |
| 81 | TCCTGCTTTT CCTCGCCGCC ACCTTGATGG CCAGCGGCAC |
| 121 | TCACGCGGCG ACCTTCACCA TCAAGAACAA CTGCGGGTTC |
| 161 | ACGGTGTGGC CGGCGGCGAC CCCAGTCGGC GGGGGCACGC |
| 201 | AGCTGAACTC AGGCGGGACG TGGACGATCA ACGTGCCGGC |
| 241 | CGGCACCAGC TCCGGCCGCG TCTGGGGCCG CACGGGCTGC |
| 281 | TCCTTCAACG GCAACAGCGG GAGCTGCCAG ACGGGCGACT |
| 321 | GCGGCGGCGC GCTCGCCTGC ACCCTCTCCG GCAAGCCTCC |
| 361 | GCTGACGCTG GCCGAGTTCA CGATCGGCGG CAGCCAGGAC |
| 401 | TTCTACGACA TCTCGGTCAT CGACGGCTTC AACATCGGCA |
| 441 | TGGCCTTCTC CTGCAGCACC GGCGTCGGGC TGGTGTGCAG |
| 481 | GGACTCGAGC TGCCCTGACG CATATCACAA TCCTCCCGAT |
| 521 | AGGAAGACCC ATGTCTGTGG CGGCAACAGC AACTACCAGG |
| 561 | TCACCTTCTG CCCGTGATGA TGAGGCAGCA AGTATATATA |
| 601 | TGCATGGCTT CTGTATCGCA TGCATGTATT TACGTATACG |
| 641 | GCACCTAGCA GGGGATGTCG GATAGGAGAG TGAATAAGAC |
| 681 | GTGTCGTGGT AGCGTACATG TCCAAGTGTG CATGCATGCA |
| 721 | TGTGCACGCG GGCATATGTA CCTGGAACTG TGTGTACTTA |
| 761 | CAGTATACTC CAGCAGTATA ATAATATGAT AAAATATAAT |
| 801 | AATAGTAAGA CACTGTCATG TCATGCTAGA AAGCAGGGAT |
| 841 | TAAAAAAAGT GAAGGTATAT TATACGGCTA CACTAGAAAG |
| 881 | CGACCTTTTG GGTCGATGGT GATAGATTCA GCCACTAGAC |
| 921 | ACACGTGTGT TTGATCGAGT CAAACAAAAG TTATATTAGG |
| 961 | CAGGACGAAT TAAAAAAATA TTTAGAGAAG ATTCTACTAC |
| 1001 | TTTACTACTT TAAGATAATT TTAACCCCTT CTTAAGTGGA |
| 1041 | GCGCCAACCT TAAAGAGAAA GATCAGAAGT AGATCATCAA |
| 1081 | GATGAAGGGG CTGCCGTCAC AAGTTGGCTC AAAGACTTCA |
| 1121 | AGACTAGCTC GGTTAATCCT TCTAATGTAG TGGAAATCTA |
| 1161 | GTCGTGTAGT TGTATTTGGT TTTCGCTGTA CTGCTATGTT |
| 1201 | TTGAACTGGT AACCCCAGTC GTGATGCTCT AATG |

A comparison of the Triticum aestivum (wheat) Tlp1 SEQ ID NO:27 sequence and the Sorghum bicolor (sorghum) Tlp1 protein SEQ ID NO:41 sequence is shown below, illustrating that the two proteins have at least 69% sequence identity.

| 69.6% identity in 168 residues overlap; Score: 629.0; Gap frequency: 2.4% | | |
|---|---|---|
| Seq27 | 6 | VLFLLLAVFAAGASAATFNIKNNCGFTIWPAGIPVGGGEALGSGQTSSINVPAGTQAGRI |
| Seq39 | 10 | LLFLAATLMASGTHAATFTIKNNCGFTVWPAATPVGGGTQLNSGGTWTINVPAGTSSGRV |
| | | *** * * **** ******** **** ***** * ** * ******* ** |
| Seq27 | 66 | WARTGCSFNGGSGSCQTGDCGGQLSCSLSGRPPATLAEYTIGGGSTQDFYDISVIDGFNL |
| Seq39 | 10 | WGRTGCSFNGNSGSCQTGDCGGALACTLSGKPPLTLAEFTIGG--SQDFYDISVIDGFNI |
| | | * ******** *********** * * *** ** **** **** ************* |
| Seq21 | 126 | AMDFSCSTGDALQCRDPSCPPPQAYQHPNDVATHACSGNNNYQITFCP |
| Seq39 | 128 | GMAFSCSTGVGLVCRDSSC--PDAYHNPPDRKTHACGGNSNYQVTFCP |
| | | * ****** * *** ** * ** * * **** ** *** **** |

An example of a Tlp1 protein from Oryza sativa Indica Group (rice) with NCBI accession number EAY83985.1 has the following sequence (SEQ ID NO:43).

| | |
|---|---|
| 1 | MASPATSSAI LVVVLVATLA AGGANAATFT ITNRCSFTVW |
| 41 | PAATPVGGGR QLSPGDTWTI NVPAGTSSGR VWGRTGCSFD |
| 81 | GSGRGSCSTG DCGGALSCTL SGQPPLTLAE FTIGGSQDFY |
| 121 | DLSVIDGFNV GMSFSCSSGV TLTCRDSSCP DAYHSPNDRK |
| 161 | THACGGNSNY QVVFCP |

A comparison of the Triticum aestivum (wheat) Tlp1 SEQ ID NO:27 sequence and the Oryza sativa Indica Group (rice) Tlp1 protein SEQ ID NO:43 sequence is shown below, illustrating that the two proteins have at least 65% sequence identity.

| 65.1% identity in 169 residues overlap; Score: 579.0; Gap frequency: 3.0% | | |
|---|---|---|
| Seq27 | 6 | VLELLLAVFAAGASAATFNIKNNCGFTIWPAGIPVGGGFALGSGQTSSINVPAGTQAGRI |
| Seq43 | 12 | VVVLVATLAAGGANAATFTITNRCSFTVWPAATPVGGGRQLSPGDTWTINVPAGTSSGRV |
| | | * * * ** **** * * * ** *** ***** * * * ******* ** |
| Seq27 | 66 | WARTGCSFNG-GSGSCQTGDCGGQLSCSLSGRPPATLAEYTIGGGSTQDFYDISVIDGEN |
| Seq43 | 12 | WGRTGCSFDGSGRGSCSTGDCGGALSCTLSGQPPLTLAEFTIGG--SQDFYDLSVIDGEN |
| | | * ****** * * *** ****** *** *** ** **** **** ***** ******* |
| Seq27 | 125 | LAMDFSCSTGDALQCRDPSCPPPQAYQHPNDVATHACSGNNNYQITFCP |
| Seq43 | 130 | VGMSFSCSSGVTLTCRDSSC--PDAYHSPNDRKTHACGGNSNYQVVFCP |
| | | * **** * * *** ** * ** *** **** ** *** *** |

An example of a Tlp1 protein from Setaria italica (foxtail millet) with NCBI accession number XP_004963268.1 has the following sequence (SEQ ID NO:44).

| | |
|---|---|
| 1 | MAASSVVVFL LLAAFVAGAS AATFTIKNNC PYTVWPAATP |
| 41 | VGGGRQLNSG QTWTLDVPAG TSSGRIWGRT GCSFSNGRGR |
| 81 | CASGDCGGAL SCTLSGQPPL TLAEFTIGSG DKQDFYDISV |
| 121 | IDGYNLPMDF SCSNGRNLQC RAPRCPDAYL FPSDNSKNHP |
| 141 | CRGNSNYRVT FCP |

An example of a nucleotide (cDNA) sequence that encodes the Setaria italica (foxtail millet) SEQ ID NO:45 Tlp1 protein (with NCBI cDNA accession number XM_004963211.1) is shown below as SEQ ID NO:45.

| | |
|---|---|
| 1 | TCACTTGTAG CAGACCAACC AGTAGTTGTC CAGTACCAAT |
| 41 | GGCGGCCTCC TCTGTCGTCG TCTTCCTCCT CCTCGCGGCC |
| 81 | TTCGTCGCCG GCGCCAGCGC GGCCACCTTC ATCATCAAGA |
| 121 | ACAACTGCCC CTATACGGTG TGGCCGGCGG CGACCCCCGT |
| 161 | CGGCGGTGGC AGGTAGCTCA ACTCAGGCCA GACGTGGACC |
| 201 | CTCGACGTGC CCGCTGGCAC CAGTTCCGGC AGGATCTGGG |
| 241 | GCCGCACCGG CTGCTCCTTC AGCAACGGCC GCGGCCGGTG |
| 281 | CGCTTCGGGC GACTGCGGCG GCGCGCTCTC CTGCACGCTC |
| 321 | TCCGGGCAGC CGCCGTTGAC TCTGGCCGAG TTCACCATCG |
| 361 | GGAGCGGCGA CAAGCAGGAC TTCTACGACA TCTCGGTGAT |
| 401 | CGACGGGTAC AACCTGCCCA TGGATTTCTC CTGCAGCAAT |
| 441 | GGCAGGAACC TGCAGTGCCG TGCCCCTCGC TGCCCCGACG |
| 481 | CGTACCTGTT CCCCAGCGAC AATTCCAAGA ACCACCCGTG |
| 521 | CCGTGGCAAC AGCAACTACA GGGTCACCTT CTGCCCATGA |
| 561 | ACGGTGGTCG AACGATGGTG CAGTAATGCA TCATCATGGC |
| 601 | CACGTACGGG AAAGAAATAA TAAGTTAATG AATGAATAAG |
| 641 | ACGACCTTTG GGTCGTGCAT GCGTGCATGC ATCTGATCTA |
| 681 | TACTATGTAC TTTCATGGAA CTTCAGTTAA TAATTTGCAC |
| 721 | GTCTTCTGCT A |

A comparison of the Triticum aestivum (wheat) Tlp1 SEQ ID NO:27 sequence and the Setaria italica (foxtail millet) Tlp1 protein SEQ ID NO:44 sequence is shown below, illustrating that the two proteins have at least 65% sequence identity.

| 65.7% identity in 172 residues overlap; Score: 6200;. Gap frequency: 0.6% | | |
|---|---|---|
| Seq27 | 2 | ATSPVLFLLLAVFAAGASAATFNIKNNCGFTIWPAGIPVGGGFALGSGQTSSINVPAGTQ |
| Seq43 | 3 | ASSVVVFLLLAAFVAGASAATFTIKNNCPYTVWPAATPVGGGRQLNSGQTWTLDVPAGTS |
| | | * * * ***** * ******** ***** * *** ***** * **** ***** |
| Seq27 | 62 | AGRIWARTGCSFNGGSGSCQTGDCGGQLSCSLSGRPPATLAEYTIGGGSTQDFYDISVID |
| Seq43 | 63 | SGRIWGRTGCSFSNGRGRCASGDCGGALSCTLSGQPPLTLAEFTIGSGDKQDFYDISVID |
| | | **** ****** * * * ***** *** *** ** **** *** * ********** |
| Seq21 | 122 | GFNLAMDFSCSTGDALQCRDPSCPPPQAYQHPNDVATHACSGNNNYQITFCP |
| Seq43 | 123 | GYNLPMDFSCSNGRNLQCRAPRCPDAYLFPSDNS-KNHPCRGNSNYRVTFCP |
| | | * ** ****** * **** * ** * * * ** ** **** |

### Transformation of Plant Cells

Plant cells can be modified to include expression cassettes or transgenes that can express any of the COI1 and/or Tlp1 proteins described herein. Such an expression cassette or transgene can include a promoter operably linked to a nucleic acid segment that encodes any of the COI1 and/or Tlp1 proteins described herein.

Promoters provide for expression of mRNA from the COI1 nucleic acids. In some cases the promoter can be a COI1 and/or Tlp1 native promoter. However, the promoter can in some cases be heterologous to the COI1 nucleic acid segment. In other words, such a heterologous promoter may not be naturally linked to such a COI1 nucleic acid segment. Instead, some expression cassettes and expression vectors can be recombinantly engineered to include a COI1 and/or Tlp1 nucleic acid segment operably linked to a heterologous promoter. A COI1 and/or Tlp1 nucleic acid is operably linked to the promoter, for example, when it is located downstream from the promoter.

A variety of promoters can be included in the expression cassettes and/or expression vectors. In some cases, the endogenous COI1 and/or Tlp1 promoter can be employed. Promoter regions are typically found in the flanking DNA upstream from the coding sequence in both prokaryotic and eukaryotic cells. A promoter sequence provides for regulation of transcription of the downstream gene sequence and typically includes from about 50 to about 2,000 nucleotide base pairs. Promoter sequences can also contain regulatory sequences such as enhancer sequences that can influence the level of gene expression. Some isolated promoter sequences can provide for gene expression of heterologous DNAs, that is a DNA different from the native or homologous DNA.

Promoters can be strong or weak, or inducible. A strong promoter provides for a high level of gene expression, whereas a weak promoter provides for a very low level of gene expression. An inducible promoter is a promoter that provides for the turning on and off of gene expression in response to an exogenously added agent, or to an environmental or developmental stimulus. For example, a bacterial promoter such as the P_{tac} promoter can be induced to vary levels of gene expression depending on the level of isothiopropylgalactoside added to the transformed cells. Promoters can also provide for tissue specific or developmental regulation. A strong promoter for heterologous DNAs can be advantageous because it provides for a sufficient level of gene expression for easy detection and selection of transformed cells and provides for a high level of gene expression when desired. In some cases, the promoter within such expression cassettes/vectors can be functional during plant development or growth.

Expression cassettes/vectors can include, but are not limited to, a promoter such as the rice actin1 (Act1) promoter. Other examples of promoters that can be used include the CaMV 35S promoter (Odell et al., Nature. 313:810-812 (1985)), or others such as CaMV 19S (Lawton et al., Plant Molecular Biology. 9:315-324 (1987)), nos (Ebert et al., Proc. Natl. Acad. Sci. USA. 84:5745-5749 (1987)). Adh1 (Walker et al., Proc. Natl. Acad. Sci. USA. 84:6624-6628 (1987)), sucrose synthase (Yang et al., Proc. Natl. Acad. Sci. USA. 87:4144-4148 (1990)), α-tubulin, ubiquitin, actin (Wang et al., Mol. Cell. Biol. 12:3399 (1992)), cab (Sullivan et al., Mol. Gen. Genet. 215:431 (1989)), PEPCase (Hudspeth et al., Plant Molecular Biology. 12:579-589 (1989)) or those associated with the R gene complex (Chandler et al., The Plant Cell. 1:1175-1183 (1989)). Further suitable promoters include the poplar xylem-specific secondary cell wall specific cellulose synthase 8 promoter, cauliflower mosaic virus promoter, the Z10 promoter from a gene encoding a 10 kD zein protein, a Z27 promoter from a gene encoding a 27 kD zein protein, inducible promoters, such as the light inducible promoter derived from the pea rbcS gene (Coruzzi et al., EMBO J. 3:1671 (1971)) and the actin promoter from rice (McElroy et al., The Plant Cell. 2:163-171 (1990)). Seed specific promoters, such as the phaseolin promoter from beans, may also be used (Sengupta-Gopalan, Proc. Natl. Acad. Sci. USA. 83:3320-3324 (1985). Other promoters useful in the practice of the invention are available to those of skill in the art.

Alternatively, novel tissue specific promoter sequences may be employed in the practice of the present invention. cDNA clones from a particular tissue can be isolated and those clones which are expressed specifically in that tissue are identified, for example, using Northern blotting. Preferably, the gene isolated is not present in a high copy number, but is relatively abundant in specific tissues. The promoter and control elements of corresponding genomic clones can then be localized using techniques well known to those of skill in the art.

Another regulatory element that the expression cassettes can have is a termination signal. Efficient expression of recombinant DNA sequences in eukaryotic cells can be enhanced by use of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 bp BamHI-BclI restriction fragment and directs both termination and polyadenylation (Sambrook, supra, at 16.6-16.7). An example of such a termination signal is a potato protease II terminator.

A COI1 and/or Tlp1 nucleic acid can be combined with the promoter by standard methods to yield an expression cassette or transgene, for example, as described in Sambrook et al. (MOLECULAR CLONING: A LABORATORY MANUAL. Second Edition (Cold Spring Harbor, N.Y.: Cold Spring Harbor Press (1989); MOLECULAR CLONING: A LABORATORY MANUAL. Third Edition (Cold Spring Harbor, N.Y.: Cold Spring Harbor Press (2000)). Briefly, a plasmid containing a promoter such as the 35S CaMV promoter can be constructed as described in Jefferson (Plant Molecular Biology Reporter 5:387-405 (1987)) or obtained from Clontech Lab in Palo Alto, Calif. (e.g., pBI121 or pBI221). Typically, these plasmids are constructed to have multiple cloning sites having specificity for different restriction enzymes downstream from the promoter. The COI1 and/or Tlp1 nucleic acids can be subcloned downstream from the promoter using restriction enzymes and positioned to ensure that the DNA is inserted in proper orientation with respect to the promoter so that the DNA can be expressed as sense or antisense RNA. Once the COI1 and/or Tlp1 nucleic acid is operably linked to a promoter, the expression cassette so formed can be subcloned into a plasmid or other vector (e.g., an expression vector).

In some embodiments, a cDNA clone encoding a COI1 and/or Tlp1 protein is synthesized, isolated, and/or obtained from a selected cell. In other embodiments, cDNA clones from other species (that encode a COI1 and/or Tlp1 protein) are isolated from selected plant tissues. For example, the nucleic acid encoding a COI1 protein can be any nucleic acid with a coding region that hybridizes to SEQ ID NO:2 and that has COI1 activity. For example, the nucleic acid encoding a Tlp1 protein can be any nucleic acid with a coding region that hybridizes to SEQ ID NO:28 and that has Tlp1 activity. In another example, the COI1 nucleic acid can encode a COI1 protein with an amino acid sequence that has at least 90%, or at least 95%, or at least 96%, or at least 97%, at least 98%, or at least 99% sequence identity to SEQ ID NO:1, 3, 5, 6, 8, 10, 12, 14, 16, 17, 19, 21, 23, or 25. In another example, the Tlp1 nucleic acid can encode a Tlp1 protein with an amino acid sequence that has at least 90%, or at least 95%, or at least 96%, or at least 97%, at least 98%, or at least 99% sequence identity to SEQ ID NO:27, 29, 31, 32, 33, 34, 36, 37, 39, 41, or 43. Using restriction endonucleases, the entire coding sequence for the COI1 and/or Tlp1 nucleic acid is subcloned downstream of the promoter in a 5′ to 3′ sense orientation.

In some cases, an endogenous COI1 and/or Tlp1 gene can be modified to generate plant cells and plants that can express increased levels of COI1 and/or Tlp1 protein(s). Mutations can be introduced into promoter regions of COI1 and/or Tlp1 loci within plant genomes by introducing targeting vectors, T-DNA, transposons, nucleic acids encoding TALENS, CRISPR, or ZFN nucleases, and combinations thereof into a recipient plant cell to create a transformed cell.

The frequency of occurrence of cells taking up exogenous (foreign) DNA can sometimes be low. However, certain cells from virtually any dicot or monocot species can be stably transformed, and these cells can be regenerated into transgenic plants, through the application of the techniques disclosed herein. The plant cells, plants, and seeds can therefore be monocotyledons or dicotyledons.

The cell(s) that undergo transformation may be in a suspension cell culture or may be in an intact plant part, such as an immature embryo, or in a specialized plant tissue, such as callus, such as Type I or Type II callus.

Transformation of the cells of the plant tissue source can be conducted by any one of a number of methods available to those of skill in the art. Examples are: Transformation by direct DNA transfer into plant cells by electroporation (U.S. Pat. No. 5,384,253 and U.S. Pat. No. 5,472,869, Dekeyser et al., The Plant Cell. 2:591 602 (1990)); direct DNA transfer to plant cells by PEG precipitation (Hayashimoto et al., Plant Physiol. 93:857 863 (1990)); direct DNA transfer to plant cells by microprojectile bombardment (McCabe et al., Bio/Technology. 6:923 926 (1988); Gordon Kamm et al., The Plant Cell. 2:603 618 (1990); U.S. Pat. No. 5,489,520; U.S. Pat. No. 5,538,877; and U.S. Pat. No. 5,538,880) and DNA transfer to plant cells via infection with Agrobacterium. Methods such as microprojectile bombardment or electroporation can be carried out with “naked” DNA where the expression cassette may be simply carried on any E. coli derived plasmid cloning vector. In the case of viral vectors, it is desirable that the system retain replication functions, but lack functions for disease induction.

One method for dicot transformation, for example, involves infection of plant cells with Agrobacterium tumefaciens using the leaf disk protocol (Horsch et al., Science 227:1229 1231 (1985). Monocots such as Zea mays can be transformed via microprojectile bombardment of embryogenic callus tissue or immature embryos, or by electroporation following partial enzymatic degradation of the cell wall with a pectinase containing enzyme (U.S. Pat. No. 5,384,253; and U.S. Pat. No. 5,472,869). For example, embryogenic cell lines derived from immature wheat embryos can be transformed by accelerated particle treatment as described by Gordon Kamm et al. (The Plant Cell. 2:603 618 (1990)) or U.S. Pat. No. 5,489,520; U.S. Pat. No. 5,538,877 and U.S. Pat. No. 5,538,880, cited above. Excised immature embryos can also be used as the target for transformation prior to tissue culture induction, selection and regeneration as described in U.S. application Ser. No. 08/112,245 and PCT publication WO 95/06128. Furthermore, methods for transformation of monocotyledonous plants utilizing Agrobacterium tumefaciens have been described by Hiei et al. (European Patent 0 604 662, 1994) and Saito et al. (European Patent 0 672 752, 1995).

Methods such as microprojectile bombardment or electroporation are carried out with “naked” DNA where the expression cassette may be simply carried on any plasmid cloning vector. In the case of viral vectors, it is desirable that the system retain replication functions, but lack functions for disease induction.

The choice of plant tissue source for transformation will depend on the nature of the host plant and the transformation protocol. Useful tissue sources include callus, suspension culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells. Selection of tissue sources for transformation of monocots is described in detail in U.S. application Ser. No. 08/112,245 and PCT publication WO 95/06128.

The transformation is carried out under conditions directed to the plant tissue of choice. The plant cells or tissue are exposed to the DNA or RNA carrying the targeting vector and/or other nucleic acids for an effective period of time. This may range from a less than one second pulse of electricity for electroporation to a 2-3 day co cultivation in the presence of plasmid bearing Agrobacterium cells. Buffers and media used will also vary with the plant tissue source and transformation protocol. Many transformation protocols employ a feeder layer of suspended culture cells (tobacco or Black Mexican Sweet corn, for example) on the surface of solid media plates, separated by a sterile filter paper disk from the plant cells or tissues being transformed.

Where one wishes to introduce DNA by means of electroporation, it is contemplated that the method of Krzyzek et al. (U.S. Pat. No. 5,384,253) may be advantageous. In this method, certain cell wall degrading enzymes, such as pectin degrading enzymes, are employed to render the target recipient cells more susceptible to transformation by electroporation than untreated cells. Alternatively, recipient cells can be made more susceptible to transformation, by mechanical wounding.

To effect transformation by electroporation, one may employ either friable tissues such as a suspension cell cultures, or embryogenic callus, or alternatively, one may transform immature embryos or other organized tissues directly. The cell walls of the preselected cells or organs can be partially degraded by exposing them to pectin degrading enzymes (pectinases or pectolyases) or mechanically wounding them in a controlled manner. Such cells would then be receptive to DNA uptake by electroporation, which may be carried out at this stage, and transformed cells then identified by a suitable selection or screening protocol dependent on the nature of the newly incorporated DNA.

A further advantageous method for delivering transforming DNA segments to plant cells is microprojectile bombardment. In this method, microparticles may be coated with DNA and delivered into cells by a propelling force. Exemplary particles include those comprised of tungsten, gold, platinum, and the like.

It is contemplated that in some instances DNA precipitation onto metal particles would not be necessary for DNA delivery to a recipient cell using microprojectile bombardment. In an illustrative embodiment, non-embryogenic cells were bombarded with intact cells of the bacteria E. coli or Agrobacterium tumefaciens containing plasmids with either the β-glucuronidase or bar gene engineered for expression in maize. Bacteria were inactivated by ethanol dehydration prior to bombardment. A low level of transient expression of the β-glucuronidase gene was observed 24-48 hours following DNA delivery. In addition, stable transformants containing the bar gene were recovered following bombardment with either E. coli or Agrobacterium tumefaciens cells. It is contemplated that particles may contain DNA rather than be coated with DNA. Hence it is proposed that particles may increase the level of DNA delivery but are not, in and of themselves, necessary to introduce DNA into plant cells.

An advantage of microprojectile bombardment, in addition to being an effective means of reproducibly stably transforming monocots, is that the isolation of protoplasts (Christou et al., PNAS. 84:3962 3966 (1987)), the formation of partially degraded cells, or the susceptibility to Agrobacterium infection is not required. An illustrative embodiment of a method for delivering DNA into maize cells by acceleration is a Biolistics Particle Delivery System, which can be used to propel particles coated with DNA or cells through a screen, such as a stainless steel or Nytex screen, onto a filter surface covered with plant cells cultured in suspension (Gordon Kamm et al., The Plant Cell. 2:603 618 (1990)). The screen disperses the particles so that they are not delivered to the recipient cells in large aggregates. It is believed that a screen intervening between the projectile apparatus and the cells to be bombarded reduces the size of projectile aggregate and may contribute to a higher frequency of transformation, by reducing damage inflicted on the recipient cells by an aggregated projectile.

For bombardment, cells in suspension are preferably concentrated on filters or solid culture medium. Alternatively, immature embryos or other target cells may be arranged on solid culture medium. The cells to be bombarded are positioned at an appropriate distance below the macroprojectile stopping plate. If desired, one or more screens are also positioned between the acceleration device and the cells to be bombarded. Through the use of techniques set forth here in one may obtain up to 1000 or more foci of cells transiently expressing a marker gene. The number of cells in a focus which express the exogenous gene product 48 hours post bombardment often range from about 1 to 10 and average about 1 to 3.

In bombardment transformation, one may optimize the prebombardment culturing conditions and the bombardment parameters to yield the maximum numbers of stable transformants. Both the physical and biological parameters for bombardment can influence transformation frequency. Physical factors are those that involve manipulating the DNA/microprojectile precipitate or those that affect the path and velocity of either the macroprojectiles or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmid DNA.

One may wish to adjust various bombardment parameters in small scale studies to fully optimize the conditions and/or to adjust physical parameters such as gap distance, flight distance, tissue distance, and helium pressure. One may also minimize the trauma reduction factors (TRFs) by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. Execution of such routine adjustments will be known to those of skill in the art.

Examples of plants, plant seeds, and/or plant cells that can have the expression systems described herein include wheat, rye, maize, millet, red wild einkorn, amaranth, bulgur, farro, maize, oats, rice, sorghum, spelt, barley, alfalfa (e.g., forage legume alfalfa), algae, apple, avocado, balsam, barley, broccoli. Brussels sprouts, cabbage, canola, cassava, cauliflower, cocoa, cole vegetables, collards, corn, cottonwood, crucifers, earthmoss, grain legumes, grasses (e.g., forage grasses), jatropa, kale, kohlrabi, maize, miscanthus, moss, mustards, nut, nut sedge, oats, oil firewood trees, oilseeds, peach, peanut, poplar, potato, radish, rape, rapeseed, rice, rutabaga, sorghum, soybean, sugar beets, sugarcane, sunflower, switchgrass, tobacco, tomato, turnips, and wheat. In some embodiments, the plant is a grain producing species. In some embodiments, the plant, plant seed, or plant cell can be a wheat plant, wheat seed, or wheat cell.

An exemplary embodiment of methods for identifying transformed cells involves exposing the bombarded cultures to a selective agent, such as an infectious agent (e.g., the causative agent of Fusarium Head Blight (FHB)), a metabolic inhibitor, an antibiotic, herbicide or the like. Cells which have been transformed and have stably integrated a marker gene conferring resistance to the selective agent used, will grow and divide in culture. Sensitive cells will not be amenable to further culturing.

To use the bar-bialaphos or the EPSPS-glyphosate selective system, bombarded tissue is cultured for about 0-28 days on nonselective medium and subsequently transferred to medium containing from about 1-3 mg/l bialaphos or about 1-3 mM glyphosate, as appropriate. While ranges of about 1-3 mg/l bialaphos or about 1-3 mM glyphosate can be employed, it is proposed that ranges of at least about 0.1-50 mg/l bialaphos or at least about 0.1-50 mM glyphosate will find utility in the practice of the invention. Tissue can be placed on any porous, inert, solid or semi-solid support for bombardment, including but not limited to filters and solid culture medium. Bialaphos and glyphosate are provided as examples of agents suitable for selection of transformants, but the technique of this invention is not limited to them.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. The R-locus is useful for selection of transformants from bombarded immature embryos. In a similar fashion, the introduction of the C1 and B genes will result in pigmented cells and/or tissues.

The enzyme luciferase is also useful as a screenable marker in the context of the present invention. In the presence of the substrate luciferin, cells expressing luciferase emit light which can be detected on photographic or X-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time.

It is further contemplated that combinations of screenable and selectable markers may be useful for identification of transformed cells. For example, selection with a growth inhibiting compound, such as bialaphos or glyphosate at concentrations below those that cause 100% inhibition followed by screening of growing tissue for expression of a screenable marker gene such as luciferase would allow one to recover transformants from cell or tissue types that are not amenable to selection alone. Slowly growing tissue was subsequently screened for expression of the luciferase gene and transformants can be identified.

### Regeneration and Seed Production

Cells that survive the exposure to the selective agent, or cells that have been scored positive in a screening assay, are cultured in media that supports regeneration of plants. One example of a growth regulator that can be used for such purposes is dicamba or 2,4-D. However, other growth regulators may be employed, including NAA, NAA+2,4-D or perhaps even picloram. Media improvement in these and like ways can facilitate the growth of cells at specific developmental stages. Tissue can be maintained on a basic media with growth regulators until sufficient tissue is available to begin plant regeneration efforts, or following repeated rounds of manual selection, until the morphology of the tissue is suitable for regeneration, at least two weeks, then transferred to media conducive to maturation of embryoids. Cultures are typically transferred every two weeks on this medium. Shoot development signals the time to transfer to medium lacking growth regulators.

The transformed cells, identified by selection or screening and cultured in an appropriate medium that supports regeneration, can then be allowed to mature into plants. Developing plantlets are transferred to soilless plant growth mix, and hardened, e.g., in an environmentally controlled chamber at about 85% relative humidity, about 600 ppm CO₂, and at about 25-250 microeinsteins/sec·m² of light. Plants can be matured either in a growth chamber or greenhouse. Plants are regenerated from about 6 weeks to 10 months after a transformant is identified, depending on the initial tissue. During regeneration, cells are grown on solid media in tissue culture vessels. Illustrative embodiments of such vessels are petri dishes and Plant Con™. Regenerating plants can be grown at about 19° C. to 28° C. After the regenerating plants have reached the stage of shoot and root development, they may be transferred to a greenhouse for further growth and testing.

Mature plants are then obtained from cell lines that have expression cassettes encoding COI1 and/or Tlp1 proteins. In some embodiments, the regenerated plants are self-pollinated. In addition, pollen obtained from the regenerated plants can be crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture can be useful if the traits are to be commercially useful.

Regenerated plants can be repeatedly crossed to inbred plants in order to introgress the mutations into the genome of the inbred plants. This process is referred to as backcross conversion. When a sufficient number of crosses to the recurrent inbred parent have been completed in order to produce a product of the backcross conversion process that is substantially isogenic with the recurrent inbred parent except for the presence of the introduced expression cassette encoding a COI1 or Tlp1 protein, the plant can be self-pollinated at least once in order to produce a homozygous backcross converted inbred containing the mutations. Progeny of these plants are in many cases true breeding.

Alternatively, seed from transformed mutant plant lines regenerated from transformed tissue cultures is grown in the field and self-pollinated to generate true breeding plants.

Seed from the fertile transgenic plants can then be evaluated for the presence of the desired COI1 and/or Tlp1 expression cassette(s), and/or the expression of the desired levels of COI1 and/or Tlp1 protein. Transgenic plant and/or seed tissue can be analyzed using standard methods such as SDS polyacrylamide gel electrophoresis, liquid chromatography (e.g., HPLC) or other means of detecting a mutation.

Once a transgenic plant with COI1 and/or Tlp1 expression cassette(s) and having pathogen resistance is identified, seeds from such plants can be used to develop true breeding plants. The true breeding plants are used to develop a line of plants with improved pathogen resistance relative to wild type, while still maintaining other desirable functional agronomic traits. Adding the mutation to other plants can be accomplished by back-crossing with this trait and with plants that do not exhibit this trait and studying the pattern of inheritance in segregating generations. Those plants expressing the target trait (e.g., pathogen resistance, good growth, good seed/kernel yield) in a dominant fashion are preferably selected. Back-crossing is carried out by crossing the original fertile transgenic plants with a plant from an inbred line exhibiting desirable functional agronomic characteristics while not necessarily expressing the trait of increased pathogen resistance and good plant growth. The resulting progeny are then crossed back to the parent that expresses the increased pathogen resistance and good plant growth. The progeny from this cross will also segregate so that some of the progeny carry the trait and some do not. This back-crossing is repeated until an inbred line with the desirable functional agronomic traits, and with expression of the trait involving an increase in pathogen resistance. Such pathogen resistance can be expressed in a dominant fashion.

The new transgenic plants can also be evaluated for a battery of functional agronomic characteristics such as growth, lodging, kernel hardness, yield, resistance to disease, resistance to insect pests, drought resistance, and/or herbicide resistance.

Plants that may be improved by these methods include but are not limited to agricultural plants of all types. Examples include grains (maize, wheat, barley, oats, rice, sorghum, amaranth, bulgur, red wild einkorn, farro, spelt, millet and rye), oil and/or starch plants (canola, potatoes, lupins, sunflower and cottonseed), forage plants (alfalfa, clover and fescue), grasses (switchgrass, prairie grass, wheat grass, sudangrass, sorghum, straw-producing plants), softwood, hardwood and other woody plants (e.g., those used for paper production such as poplar species, pine species, and eucalyptus). Plants useful for making biofuels and ethanol include corn, grasses (e.g., miscanthus, switchgrass, and the like), as well as trees such as poplar, aspen, willow, and the like. Plants useful for generating dairy forage include legumes such as alfalfa, as well as forage grasses such as bromegrass, and bluestem. In some embodiments the plant is a gymnosperm. Examples of plants useful for grain production, include wheat, rye, maize, millet, red wild einkorn, amaranth, bulgur, farro, maize, oats, rice, sorghum, spelt, and barley.

### Determination of Stably Transformed Plant Tissues

To confirm the presence of COI1, Tlp1 and/or expression cassettes encoding COI1 and/or Tlp1 proteins in the regenerating plants, or seeds or progeny derived from the regenerated plant, a variety of assays may be performed. Such assays include, for example, molecular biological assays available to those of skill in the art, such as Southern and Northern blotting and PCR; biochemical assays, such as detecting the presence of a protein product, e.g., by immunological means (ELISAs and Western blots) or by enzymatic function; plant part assays, such as leaf, seed or root assays; and also, by analyzing the phenotype of the whole regenerated plant.

Whereas DNA analysis techniques may be conducted using DNA isolated from any part of a plant, RNA may only be expressed in particular cells or tissue types and so RNA for analysis can be obtained from those tissues. PCR techniques may also be used for detection and quantification of RNA produced from introduced expression cassettes encoding COI1 and/or Tlp1 proteins. For example, PCR also be used to reverse transcribe RNA into DNA, using enzymes such as reverse transcriptase, and then this DNA can be amplified through the use of conventional PCR techniques.

For example, if no amplification of COI1 and/or Tlp1 mRNAs is observed, then an expression cassette encoding COI1 protein and/or Tlp1 protein may not have been successfully introduced. Information about introduced expression cassettes can also be obtained by primer extension or single nucleotide polymorphism (SNP) analysis.

Further information about the nature of the RNA product may be obtained by Northern blotting. This technique will demonstrate the presence of an RNA species and give information about the integrity of that RNA. The presence or absence of an RNA species (e.g., COI1 and/or Tlp1 RNA) can also be determined using dot or slot blot Northern hybridizations. These techniques are modifications of Northern blotting and also demonstrate the presence or absence of an RNA species.

While Southern blotting and PCR may be used to detect the expression cassettes encoding COI1 and/or Tlp1 proteins, they do not provide information as to whether the preselected DNA segment is being expressed. Expression may be evaluated by specifically identifying the protein products of the introduced COI1 and/or Tlp1 expression cassette, by detecting expression of the COI1 and/or Tlp1 proteins, or evaluating the phenotypic changes brought about by introduction of such proteins.

Assays for the production and identification of specific proteins may make use of physical-chemical structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange, liquid chromatography or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products, or the absence thereof, that have been separated by electrophoretic techniques. Additional techniques may be employed to absolutely confirm COI1 and/or Tlp1 mRNA or protein expression. Amino acid sequencing following purification can also be employed. The Examples of this application also provide assay procedures for detecting and quantifying infection and plant growth. Other procedures may be additionally used.

The expression of a gene product can also be determined by evaluating the phenotypic results of its expression. These assays also may take many forms including but not limited to analyzing changes in the resistance to infection, resistance to herbicides, growth characteristics, or other physiological properties of the plant. Expression of selected DNA segments encoding different amino acids or having different sequences and may be detected by amino acid analysis or sequencing.

### Definitions

The term “heterologous” when used in reference to a nucleic acid or protein refers to a nucleic acid or protein that has been manipulated in some way. For example, a heterologous nucleic acid includes a nucleic acid from one species introduced into another species. A heterologous nucleic acid also includes a nucleic acid that is native to an organism that has been altered in some way (e.g., mutated, added in multiple copies, present in a locus within the genome, expressed from an autonomously replicating vector, linked to a non-native promoter, linked to a mutated promoter, or linked to an enhancer sequence, etc.). Heterologous nucleic acids may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). In some cases, heterologous nucleic acids are distinguished from endogenous plant genes in that the heterologous nucleic acids can be joined to nucleotide sequences comprising regulatory elements such as promoters that are not found naturally associated with the nucleic acid. In another example, the heterologous nucleic acids are associated with portions of the chromosome not found in nature (e.g., genes expressed in loci where the gene is not normally expressed).

The term “nucleic acid,” “nucleic acid segment” or “nucleic acid of interest” refers to any RNA or DNA, where the manipulation of which may be deemed desirable for any reason (e.g., treat or reduce the incidence of disease, confer improved qualities, etc.), by one of ordinary skill in the art. Such nucleic acids include, but are not limited to, coding sequences of structural genes (e.g., disease resistance genes, reporter genes, selection marker genes, oncogenes, drug resistance genes, growth factors, etc.), and noncoding regulatory sequences which do not encode an mRNA or protein product (e.g., promoter sequence, polyadenylation sequence, termination sequence, enhancer sequence, etc.).

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

The term “Tₘ” refers to the “melting temperature” of a nucleic acid. The melting temperature is the temperature at which a population of double-stranded nucleic acid molecules becomes half dissociated into single strands. The equation for calculating the Tₘ of nucleic acids is available to those of skill in the art. As indicated by standard references, a simple estimate of the Tₘ value may be calculated by the equation: Tₘ=81.5+0.41 (% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

The term “stringency” refers to the conditions of temperature, ionic strength, and the presence of other compounds such as organic solvents, under which nucleic acid hybridizations are conducted. With “high stringency” conditions, nucleic acid base pairing will occur between nucleic acid fragments that have a high frequency of complementary base sequences. Thus, conditions of “low” stringency are often required with nucleic acids that are derived from organisms that are genetically diverse, as the frequency of complementary sequences is usually less. Stringency conditions are substantially determined by wash conditions (and not by hybridization conditions).

“Low stringency conditions” when used in reference to nucleic acid hybridization comprise conditions equivalent to binding or hybridization at 42° C. in a solution consisting of 5×SSPE (43.8 g/l NaCL 6.9 g/l NaH₂PO₄H₂O and 1.85 g/l EDTA. pH adjusted to 7.4 with NaOH), 0.1% SDS, 5×Denhardt's reagent (50×Denhardt's contains per 500 ml: 5 g Ficoll (Type 400, Pharmacia), 5 g BSA (Fraction V; Sigma)) and 100 μg/ml denatured salmon sperm DNA. Washing conditions that substantially determine whether “low stringency” hybridization occurs, include washing in a solution comprising 5×SSPE, 0.1% SDS at 42° C., for example, when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA. pH adjusted to 7.4 with NaOH), 0.5% SDS, 5×Denhardt's reagent and 100/μg/ml denatured salmon sperm DNA. Washing conditions that substantially determine whether “medium stringency” hybridization occurs, include washing in a solution comprising 1.0×SSPE, 1.0% SDS at 50° C. when a probe of about 500 nucleotides in length is employed.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA. Washing conditions that substantially determine whether “high stringency” hybridization occurs include washing in a solution comprising 0.1×SSPE, 1.0% SDS at 60° C. when a probe of about 500 nucleotides in length is employed.

The term “expression” or “express” when used in reference to a nucleic acid, refers to the process of converting genetic information encoded in a nucleic acid into RNA (e.g., mRNA, rRNA, tRNA, or snRNA) through “transcription” of the nucleic acid (i.e., via the enzymatic action of an RNA polymerase). In some cases, “expression” or “express” can include translation into protein (as when a gene encodes a protein), through “translation” of mRNA. Expression can be regulated at many stages in the process. “Up-regulation” or “activation” refers to regulation that increases the production of nucleic acid expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “operably linked” refers to the linkage of nucleic acid segments in such a manner that a regulatory nucleic acid segment capable of directing the transcription of a given nucleic acid segment (e.g., a coding region) and/or the synthesis of a desired protein molecule is produced. The term also refers to the linkage of amino acid sequences in such a manner so that a functional protein is produced.

The term “regulatory element” refers to a genetic element that controls some aspect of nucleic acid expression. For example, a promoter is a regulatory element that facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short nucleic acid segments that interact specifically with cellular proteins involved in transcription (Maniatis. et al., Science 236:1237 (1987), herein incorporated by reference). Promoter and enhancer elements have been isolated from a variety of prokaryotic (e.g., bacterial) and eukaryotic sources. Eukaryotic promoter and enhancer elements can be obtained, for example, from genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Maniatis, et al., supra (1987), herein incorporated by reference). The terms “promoter element.” “promoter,” or “promoter sequence” refer to a nucleic acid segments that are generally located at the 5′ end (i.e. precedes) of the coding region of nucleic acid. The location of most promoters known in nature precedes the transcribed region. The promoter functions as a switch, activating the expression of an encoded product (e.g., an RNA). If a gene or expression cassette is activated, it can be transcribed, or participate in transcription. Transcription involves the synthesis of RNA from the gene. The promoter, therefore, serves as a transcriptional regulatory element and also provides a site for initiation of transcription of the gene into mRNA.

Promoters may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue (e.g., leaves). Tissue specificity of a promoter may be evaluated by; for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected.

The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody that is specific for the polypeptide product encoded by the nucleotide sequence of interest whose expression is controlled by the promoter. A labeled (e.g., peroxidase conjugated) secondary antibody that is specific for the primary antibody is allowed to bind to the sectioned tissue and specific binding detected (e.g., with avidin/biotin) by microscopy.

Promoters may be “constitutive” or “inducible.” The term “constitutive” when made in reference to a promoter means that the promoter is capable of directing transcription of an operably linked nucleic acid in the absence of a stimulus (e.g., in the absence of heat shock, chemical inducers, light, etc.). Typically, constitutive promoters are capable of directing expression of a transgene in substantially any cell and any tissue. Exemplary constitutive plant promoters include, but are not limited to SD Cauliflower Mosaic Virus (CaMV SD; see e.g., U.S. Pat. No. 5,352,605, incorporated herein by reference), mannopine synthase, octopine synthase (ocs), superpromoter (see e.g., WO 95/14098, herein incorporated by reference), and ubi3 promoters (see e.g., Garbarino and Belknap, Plant Mol. Biol. 24:119-127 (1994), herein incorporated by reference). Such promoters have been used successfully to direct the expression of heterologous nucleic acid sequences in transformed plant tissue.

In contrast, an “inducible” promoter is one that is capable of directing a level of transcription of an operably linked nucleic acid sequence in the presence of a stimulus (e.g., heat shock, chemical inducers, light, etc.) that is different from the level of transcription of the operably linked nucleic acid sequence in the absence of the stimulus.

The following Examples describe some of the experiments performed in the development of the invention.

### Example 1: Materials and Methods

This Example describes some of the materials and methods employed in the development of the invention.

### Construct Assembly

Construct pjBarTlp and pjBarCoi: The wheat 674 bp tlp1 GenBank accession number X58394 and wheat 2167 bp Coi1 GenBank accession number HM447645 genes were synthesized. The cDNAs encoding these Tlp1 and Coi1 proteins were cloned into the Pjs101 plasmid (Nguyen et al. 2013). The Pjs101 contains two linked cassettes, one containing the bacterial mannitol-1-phosphate dehydrogenase (mtlD) gene regulated by the rice actin promoter (Act1) and the potato protease inhibitor II terminator; and the other cassette containing the bar herbicide resistance selectable marker gene regulated by the 35S promoter and Nos terminator. A new construct was developed by eliminating the mtlD gene using Xba1 and inserting the synthesized tlp1 or coi1 respectively.

### Plant Material and Genetic Transformation

Wheat cv. Bobwhite plants were grown to maturity from seeds in greenhouses. Immature embryos were isolated and cultured in-vitro following Zhang et al. (2000).

### Primary

transformants were transferred to the growth chamber and tested for herbicide resistance using leaf painting assay with a 0.1% aqueous Liberty™ solution containing 18.9% glufosinate ammonium.

### Polymerase Chain Reaction (PCR)

Genomic DNAs were extracted from herbicide resistant plantlets, as well as the wild type control plants using the CTAB method (Xin and Chin 2012). PCR was performed by the amplifying of a part of the rice actin promoter and a part of tlp1 gene, using forward rice Actin primer and reverse primer of either tlp1 for detecting the pjBarTlp construct integration; or coi1 to detect pjBarCoi construct with expected size of (˜400 and 696 bp for Tlp1 and Coi1 respectively) as well as for the bar gene (400 bp) using specific primers (Table 1).

TABLE 1
Primer Sequences

| | | Product |
|---|---|---|
| Primer name | Sequence | length |
| UBC Forward | 5′ CCGTTTGTAGAGCCATAATTGCA 3′ | 76 |
| | (SEQ ID NO: 46) | |
| UBC Reverse | 5' AGGTTGCCTGAGTCACAGTTAAGTG 3′ | |
| | (SEQ ID NO: 47) | |
| Tlp1 Forward | 5′ cgctgaccgagaacaccat 3′ (SEQ ID NO: 48) | 58 |
| Tlp1 Reverse | 5′ tcgatcaccgagatgtcgtaga 5′ (SEQ. ID NO: 49) | |
| Coi1 Forward | 5′ tggcgtactactcccatct 3′ (SEQ ID NO: 50) | 71 |
| Coi1 Reverse | 5′ gagacaccataccggcttaatg 3′ (SEQ ID NO: 51) | |

qPCR

Total RNA was extracted from transgenic as well as the control non-transgenic plants using total RNA isolation system (EZNA plant RNA Kit—Omega bio-tec, USA). Expression of the integrated transgene was tested on the RNA extracted from herbicide resistant T1 plants. First, the strand cDNA was synthesized using Goscript Reverse Transcriptase (Promega cat no. A5003)

SYBR Green Real-time RT-PCR was conducted on the resulting cDNA. The total reaction (20 μl) contained 10 ng cDNA, 0.3 μM of each primer, and 1× Fast SYBR green master Mix (Applied Biosystems, USA). Reactions were performed using ABI 7900 HT RT-PCR system (Applied Biosystems, USA) under the conditions of: 94° C. for 30 s, 40 cycles of 95° C. for 15 s, 60° C. for 60 s and 72° C. for 20 s to calculate cycle threshold (Ct) values. The ubiquitin-conjugating enzyme E2 gene was used as a housekeeping gene to standardize the gene expression. Relative Quantization of Gene Expression method ΔΔct was used to analyze the results using 2^{-ΔΔCT} formula. The primer sequences employed are listed in Table 1.

### Example 2: Expression of Tlp1 and Coi1 in Transgenic Plant Lines

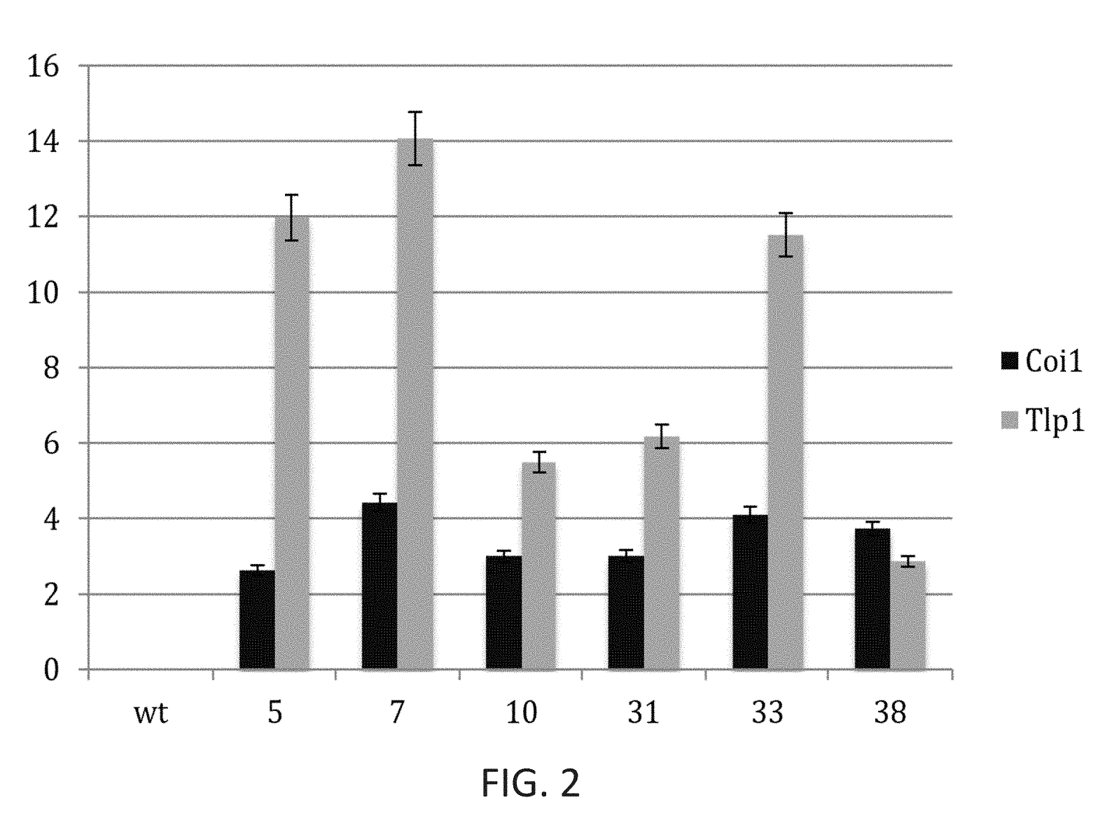

The qPCR analysis showed that among forty-four first generation (T0) independent transgenic lines with the selectable marker bar gene and at least one of the two constructs, only six lines exhibited expression of both tlp1 and Coi1 genes. The level of expression of these two genes in these six independently transformed lines is shown in FIG. 2.

### Example 2: FHB Symptoms Reduced in Plants Over-Expressing TLP1 and COI1

The Example illustrates that over-expression of TLP1 and COI1 reduces the symptoms of FHB in transgenic wheat plant lines that over-express TLP1 and COI1.

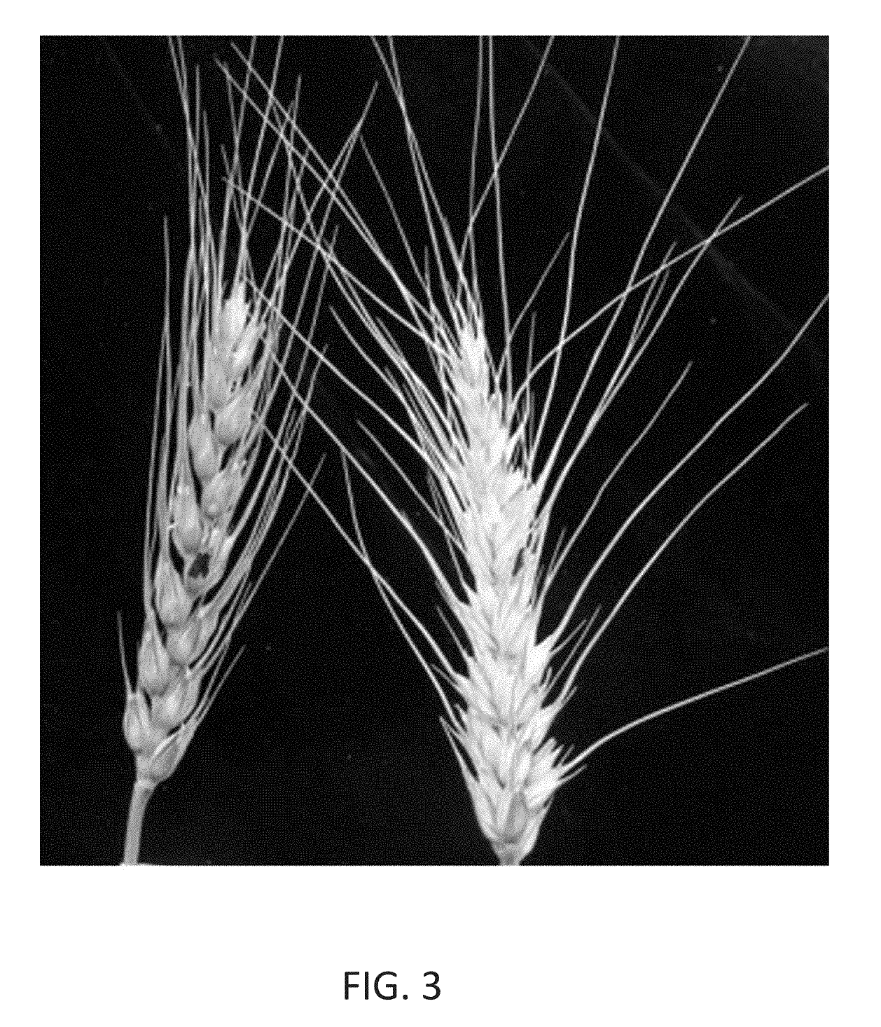

FIG. 3 illustrates the symptoms of the FHB pathogen (Fusarium graminearum) cell-free mycotoxin after single spot microinjection into wild type control spike (left) versus the first generation (T0) TLP and COI1 over-expressed spike (right) 21 days after inculcation. The site of injection is shown as a single black spot on each spike. Note that the T0 plants spikes are expected to be smaller than their control plant spikes, but T1 plants will have the normal size spikes.

Table 2 illustrates differences in the percentage of FHB infection after 21 days of point inoculation in non-transgenic Bobwhite wheat plants compared to six different independent transgenic plant lines that over-expressed of TLP1 and COI1.

TABLE 2
Percent Infection

| Plant line | % Infection |
|---|---|
| WT | 0.95 ± 0.040 |
| 5 | 0.05 ± 0.027 |
| 7 | 0.04 ± 0.020 |
| 10 | 0.01 ± 0.008 |
| 31 | 0.01 ± 0.007 |
| 33 | 0.02 ± 0.013 |
| 38 | 15 ± 0.008 |

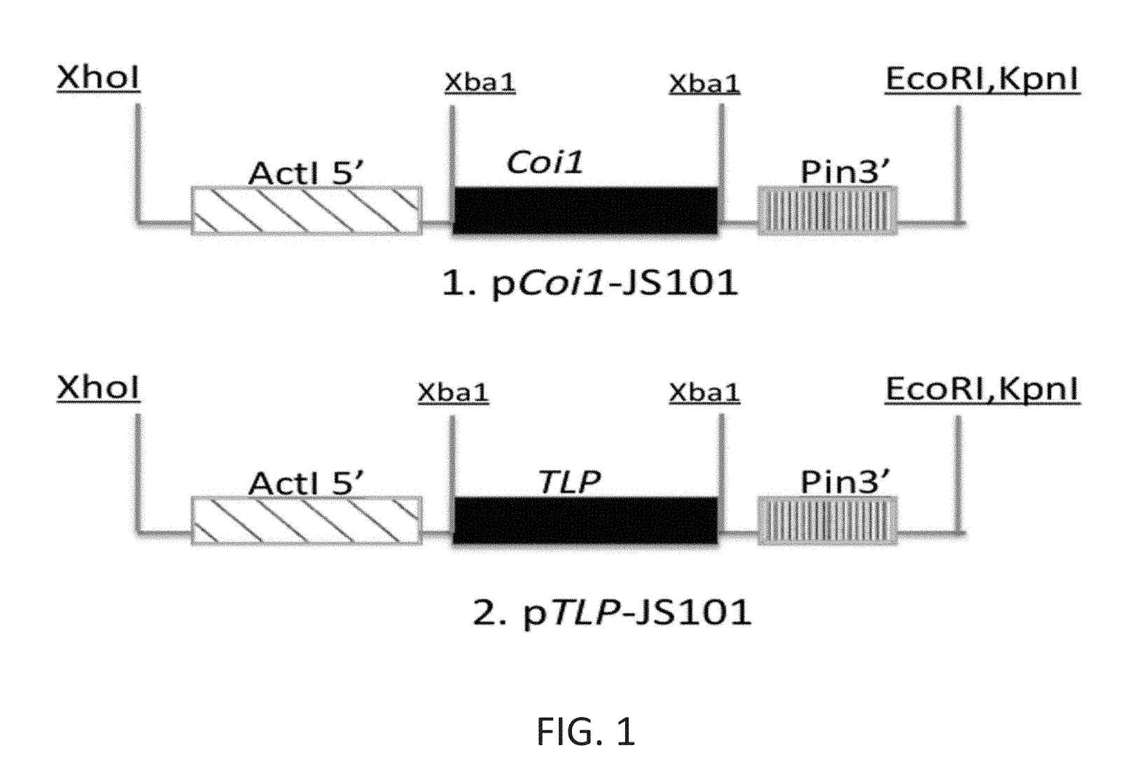

Disease assessment was conducted at three different time points, at one week, two weeks, and three weeks. Disease progression was recorded for each inoculated spike by counting the healthy seeds in both sides (up and down) from the point of inoculation (see e.g., FIG. 1). The area under the disease progress curve (AUDPC) was calculated by inserting the infection percentage into the Shaner and Finney equation (Shaner G. and Finney R. E., Weather and Epidemics of Septoria leaf blotch of wheat. Phytopathol. 66:781-785 (1976)) to describe the increase of wild type plant susceptibility.

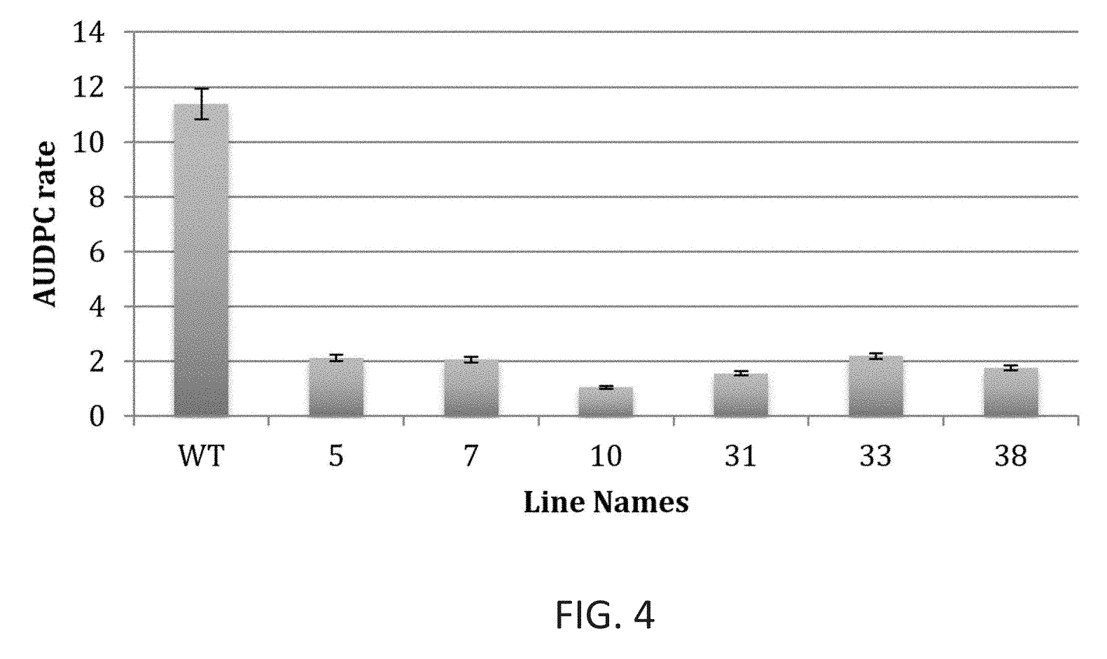

The AUDPC is graphically illustrated in FIG. 4. As illustrated, there was a significant difference between the AUDPC for the transgenic lines that over-express TLP1 and COI1 compared to Bobwhite wild type plants (ANOVA P<0.01). The responses of the individual transgenic lines were different. Transgenic plant line no. 1 exhibited the most resistance to FHB. Table 3 shows the Dependent Variable: measurements based on ANOVA, Least Significant Difference (LSD) between the non-transgenic Bobwhite wheat plants and six different independent transgenic lines showing a significant differences at the 0.05 level.

TABLE 3
Statistical Analysis of Wild Type vs. Transgenic Plant Lines

| | | | | | 95% | 95% |
|---|---|---|---|---|---|---|
| | | Mean | | | Confidence | Confidence |
| | | Difference | Std. | | Interval | Interval |
| | group | (I-J) | Error | Sig. | lower | higher |
| WT | 3 | −21.56333* | 8.06537 | .018 | −38.8618 | −4.2648 |
| | 9 | −32.40000* | 8.06537 | .001 | −49.6985 | −15.1015 |
| | 10 | −32.46333* | 8.06537 | .001 | −49.7618 | −15.1648 |
| | 31 | −39.38333* | 8.06537 | .000 | −56.6818 | −22.0848 |
| | 33 | −34.55333* | 8.06537 | .001 | −51.8518 | −17.2548 |
| | 38 | −40.15333* | 8.06537 | .000 | −57.4518 | −22.8548 |

### Example 3: Plants Over-Expressing TLP1 and COI1

The Example illustrates that over-expression of TLP1 and COI1 does not adversely affect plant growth and seed production.

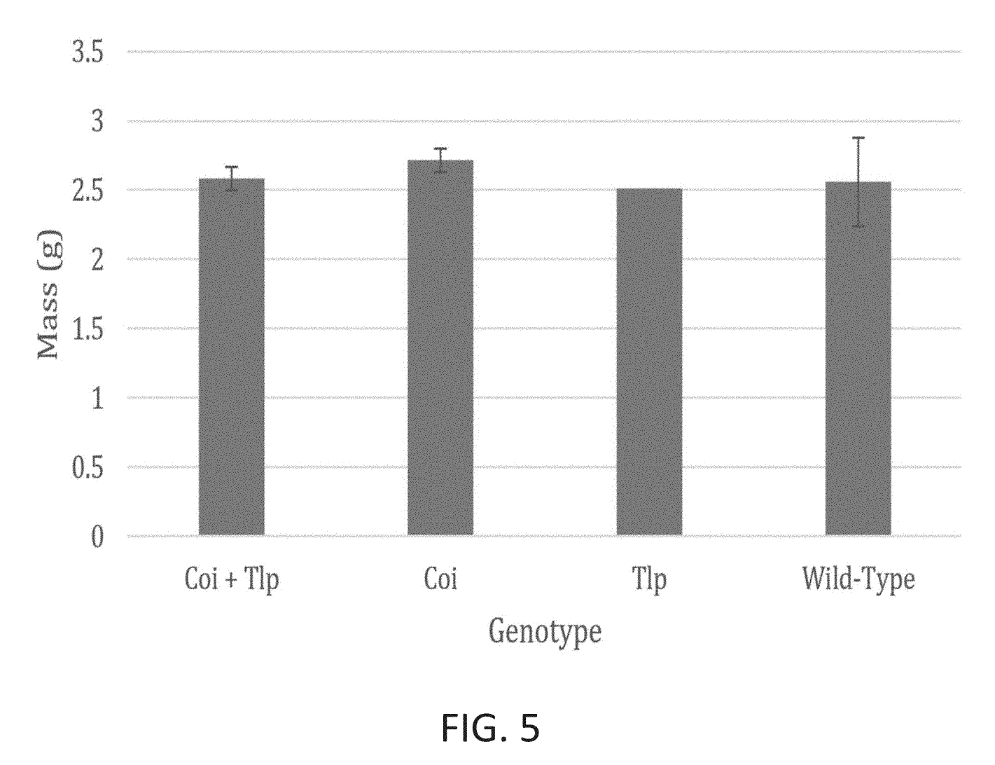

FIG. 5 graphically illustrates the mass of 100 seeds from wild type wheat plants (rightmost bar) compared to the mass of 100 seeds from transgenic plants that overexpress COI1 and TLP (leftmost bar), the mass of 100 seeds from transgenic plants that overexpress COI1 (second from the left bar), and the mass of 100 seeds from transgenic plants that overexpress TLP (third from the left bar). As shown the mass of seeds from transgenic plants that overexpress COI1 and TLP is about the same as observed for wild type.

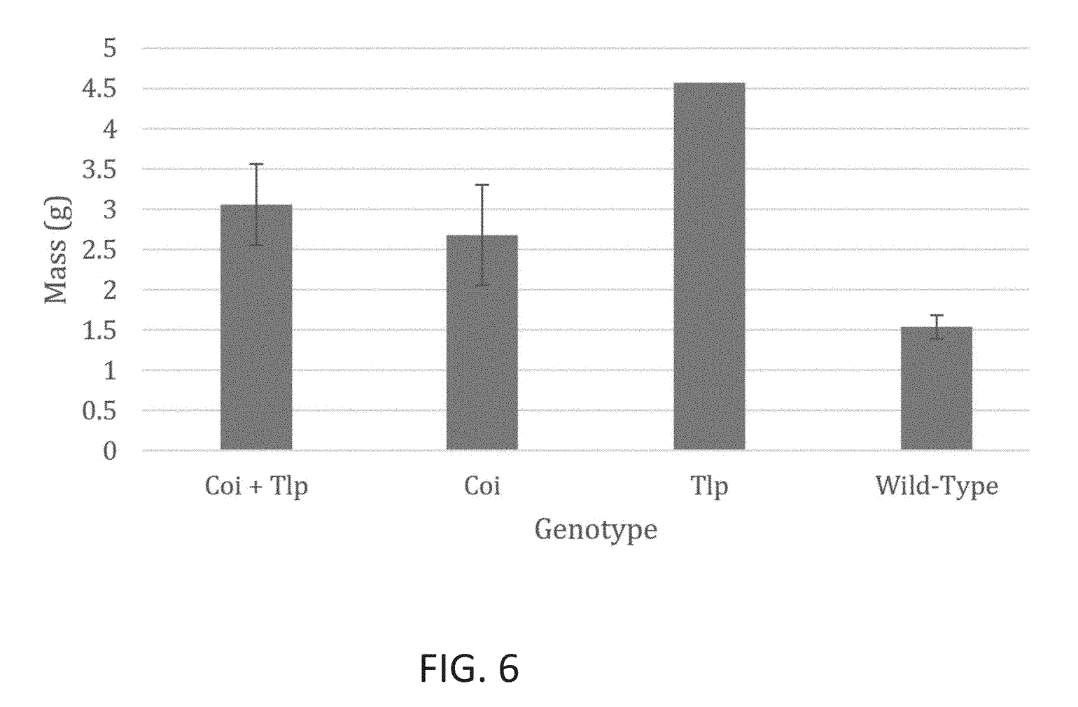

FIG. 6 illustrates the estimated mass per plant of transgenic plants that overexpress COI1 and TLP (leftmost bar), transgenic plants that overexpress COI11 (second from the left bar), and transgenic plants that overexpress TLP (third from the left bar), compared to the mass per plant of wild type plants. As illustrated, the estimated mass per plant of transgenic plants that overexpress COI1 and TLP is actually greater than the estimated mass per plant of wild type plants.

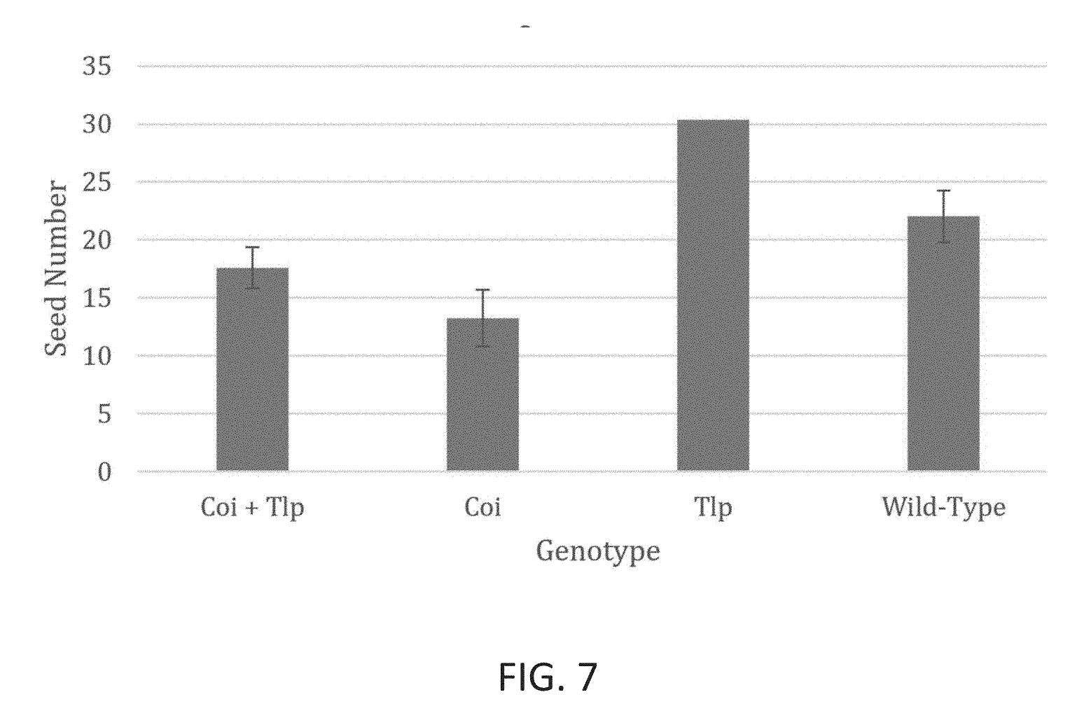

FIG. 7 graphically illustrates the seed numbers per head of transgenic plants that overexpress COI1 and TLP (leftmost bar), transgenic plants that overexpress COI1 (second from the left bar), and transgenic plants that overexpress TLP (third from the left bar), compared to the seed numbers per head per plant of wild type plants. As illustrated, the seed numbers per head of transgenic plants that overexpress COI1 and TLP is statistically equivalent to the seed numbers per head of wild type plants.

### Example 4: Plants Over-Expressing TLP1 and COI1

Transgenic wheat lines were generated containing the genes COI and TLP singly and in combination. Transgenic plants were generated from these plant lines. Transgenic plants were inoculated with Fusarium head blight (FHB) isolates, and the infected plants were maintained in greenhouse facilities for conducting disease assays and consultation.

Approximately 500 individual wheat heads and whole plants were evaluated, including thirty-one (31) lines with COI+TLP, seventeen (17) lines carrying COI, and one (1) line carrying TLP transgenes.

FHB resistance data was obtained for each line as illustrated in Table 4 below.

TABLE 4
Average Percent FHB Infection

| | Percent |
|---|---|
| Gene | Infection |
| COI + TLP | 0.181 |
| COI | 0.183 |
| TLP | 0.200 |

As illustrated, in this study overexpression of COI and COI+TLP provides greater resistance than overexpression of TLP alone.

### REFERENCES

Anand A., T. Zhou, H. N. Trick, B. S. Gill, W. W. Bockus, S. Muthukrishnan (2003). Greenhouse and field testing of transgenic wheat plants stably expressing genes for thaumatin-like protein, chitinase and glucanase against Fusarium gramninearum. J Exp Bot 2003, 54(384):1101-1111.
Becker, D., Brettschneider, R., & Lörz, H. (1994). Fertile transgenic wheat from microprojectile bombardment of scutellar tissue. The Plant Journal. 5(2), 299-307.
Buerstmayr, H., Ban. T., & Anderson, J. A. (2009). QTL mapping and marker-assisted selection for Fusarium head blight resistance in wheat: a review. Plant breeding, 128(1). 1-26.
Chen, W. P., Chen, P. D., Liu, D. J., Kynast, R., Friebe, B., Velazhahan, R., . . . & Gill, B. S. (1999). Development of wheat scab symptoms is delayed in transgenic wheat plants that constitutively express a rice thaumatin-like protein gene. Theoretical and Applied Genetics, 99(5), 755-760.
Chen, Y., Gao, Q., Huang, M., Liu, Y., Liu, Z., Liu, X., & Ma, Z. (2015). Characterization of RNA silencing components in the plant pathogenic fungus Fusarium graminearum. Scientific reports. 5.
Di, R., Blechl, A., Dill-Macky, R., Tortora, A., & Tumer, N. E. (2010). Expression of a truncated form of yeast ribosomal protein L3 in transgenic wheat improves resistance to Fusarium head blight. Plant science, 178(4), 374-380.
Ferrari, S., Sella, L., Janni, M., De Lorenzo, G., Favaron, F., & D'Ovidio, R. (2012). Transgenic expression of polygalacturonase-inhibiting proteins in Arabidopsis and wheat increases resistance to the flower pathogen Fusarium graminearum. Plant Biology, 14(s1), 31-38.
Han, J., Lakshman, D. K., Galvez, L. C., Mitra, S., Baenziger, P. S., & Mitra, A. (2012). Transgenic expression of lactoferrin imparts enhanced resistance to head blight of wheat caused by Fusarium graminearum. BMC plant biology, 12(1), 33.
Chen W. P., P. D. Chen, D. J. Liu, R. Kynast, B. Friebe, R. Velazhahan, S. Muthukrishnan, B. S. Gill (1999). Development of wheat scab symptoms is delayed in transgenic wheat plants that constitutively express a rice thaumatin-like protein gene. Theor Appl Genet. 99(5):755-760.
Jia G., P. Chen, G. Qin, G. Bai, X. Wang, S. Wang, B. Zhou, S. Zhang, D. Liu et al. QTLs for Fusarium head blight response in a wheat DH population of Wangshuibai/Alondra‘s’. Euphytica. 146(3): 183-191.
Li, X., Shin, S., Heinen, S., Dill-Macky, R., Berthiller, F., Nersesian, N., . . . & Muchlbauer, G. J. (2015). Transgenic wheat expressing a barley UDP-glucosyltransferase detoxifies deoxynivalenol and provides high levels of resistance to Fusarium graminearum. Molecular Plant-Microbe Interactions. MPMI-03.
Li, Z., Zhou, M., Zhang, Z., Ren, L., Du, L., Zhang, B., . . . & Xin, Z. (2011). Expression of a radish defensin in transgenic wheat confers increased resistance to Fusarium graminearum and Rhizoctonia cerealis. Functional & integrative genomics, 11(1), 63-70.
Li G L, Y. Yen Y (2008). Jasmonate and ethylene signaling pathway may mediate Fusarium head blight resistance in wheat. Crop Sci. 48(5): 1888-1896.
Liu S. X., M. O. Pumphry, B. S. Gill, H. N. Trick, J X Zhang, J. Dlezel, B. Chalhoub, J. A Anderson (2008). Toward positional cloning of fhb1, a major QTL for Fusarium head blight resistance in wheat. Cereal Research Commun. 36: 195-201.
Mackintosh C. A., J. Lewis, L. E. Radmer, S. Shin, S. J. Heinen, L. A. Smith, M. N. Wyckoff, C. K. Dill-Macky, C. K. Evans, S. Kravchenko (2007). Overexpression of defense response genes in transgenic wheat enhances resistance to Fusarium head blight. Plant Cell Rep 2007, 26(4):479-488.
Muthukrishnan, D. J. Liu, P. D. Chen, B. S. Gill (2001). Isolation and characterization of novel cDNA clones of acidic chitinases and β-1,3-glucanases from wheat spikes infected by Fusarium graminearum. Theor Appl Genet. 102(2-3):353-362.
Makandar, R., Essig, J. S., Schapaugh, M. A., Trick, H. N., & Shah, J. (2006). Genetically engineered resistance to Fusarium head blight in wheat by expression of Arabidopsis NPR1. Molecular Plant-Microbe Interactions, 19(2), 123-129.
Makandar, R., Nalam, V., Chaturvedi, R., Jeannotte, R., Sparks, A. A., & Shah, J. (2010). Involvement of salicylate and jasmonate signaling pathways in Arabidopsis interaction with Fusarium graminearum. Molecular plant-microbe interactions, 23(7), 861-870.
Nalam, V. J., Alam, S., Keereetaweep, J., Venables, B., Burdan, D., Lee, H., . . . & Shah, J. (2015). Facilitation of Fusarium graminearum Infection by 9-lipoxygenases in Arabidopsis and Wheat. Molecular Plant-Microbe Interactions, 28(10), 1142-1152.
Nehra, N. S., Chibbar, R. N., Leung, N., Caswell, K., Mallard, C., Steinhauer, L., . . . & Kartha, K. K. (1994). Self-fertile transgenic wheat plants regenerated from isolated scutellar tissues following microprojectile bombardment with two distinct gene constructs†. The Plant Journal. 5(2), 285-297.
Nguyen, T. X., Nguyen, T., Alameldin, H., Goheen, B., Loescher, W., & Sticklen, M. (2013). Transgene Pyramiding of the HVA1 and mtlD in T3 Maize (Zea mays L.) plants confers drought and salt tolerance, along with an increase in crop biomass. International Journal of Agronomy. Online: 10 pages. http://dx.doi.org/10.1155/2013/598163
Okubara, P., Blechl, A., McCormick, S., Alexander, N., Dill-Macky, R., & Hohn, T. (2002). Engineering deoxynivalenol metabolism in wheat through the expression of a fungal trichothecene acetyltransferase gene. Theoretical and Applied Genetics, 106(1), 74-83.
Pierson A, S. Mimoun, L. S. Murate, N. Loiseau, Y. Lippi, A. F. Bracarense, L. Liaubet, G. Schatzmayr, F. Berthiller, W. D. Moll and Oswald (2015). Intestinal toxicity of the masked mycotoxin deoxynivalenol-3-β-D-glucoside. Arch Toxicol. 9: 24: 1-10.
Pritsch C, C. P. Vance, W. R. Bushnell, D. A. Somers, T. M. Hohn, G. J. Muehlbauer (2001). Systemic expression of defense response genes in wheat spikes as a response to Fusarium graminearum infection. Physiol Mol Plant. 58(1): 1-12.
Thomma, B. P., Eggermont, K., Penninckx, I. A., Mauch-Mani, B., Vogelsang, R., Cammue, B. P., & Broekaert, W. F. (1998). Separate jasmonate-dependent and salicylate-dependent defense-response pathways in Arabidopsis are essential for resistance to distinct microbial pathogens. Proceedings of the National Academy of Sciences, 95(25), 15107-15111.
Vasil, V., Castillo, A. M., Fromm, M. E., & Vasil, I. K. (1992). Herbicide resistant fertile transgenic wheat plants obtained by microprojectile bombardment of regenerable embryogenic callus. Nature Biotechnology, 10(6), 667-674.
Volpi, C., Janni, M., Lionetti, V., Bellincampi, D., Favaron, F., & D'Ovidio, R. (2011). The ectopic expression of a pectin methyl esterase inhibitor increases pectin methyl esterification and limits fungal diseases in wheat. Molecular Plant-Microbe Interactions, 24(9), 1012-1019.
Weeks, J. T., Anderson, O. D., & Blechl, A. E. (1993). Rapid production of multiple independent lines of fertile transgenic wheat (Triticum aestivum). Plant Physiology, 102(4), 1077-1084.
Xiao J., X. Jin, X. Ja, H. Wang, A Cao, W. Zhao, H. Pei, Z. Xue, L. He, Q. Chen and X. Wang (2013). Transcriptome-based discovery of pathways and genes related to resistance against Fusarium head blight in wheat landrace and Wangshuibai. BMC Genomics, 14: 197-217.
Xin, Z., & Chen, J. (2012). A high throughput DNA extraction method with high yield and quality. Plant methods, 8(1), 1-7.
Xing, L., Wang, H., Jiang, Z., Ni, J., Cao, A., Yu, L., & Chen, P. (2008). Transformation of wheat thaumatin-like protein gene and diseases resistance analysis of the transgenic plants. Acta Agronomica Sinica, 34(3), 349.
Zhang, L., Rybczynski, J. J., Langenberg, W. G., Mitra, A., & French, R. (2000). An efficient wheat transformation procedure: transformed calli with long-term morphogenic potential for plant regeneration. Plant Cell Reports, 19(3), 241-250.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements

1. An expression system comprising an expression cassette comprising at least one promoter operably linked to a nucleic acid that encodes a COI1 protein, a Tlp1 protein, or a combination thereof.
2. The expression system of statement 1, comprising two expression cassettes, a first expression cassette comprising a first promoter operably linked to a nucleic acid that encodes a COI1 protein, and a second promoter operably linked to a nucleic acid that encodes a Tlp1 protein.
3. The expression system of statement 1 or 2, wherein the COI1 protein has a sequence with at least 95% sequence identity to SEQ ID NO:1, 3, 5, 6, 8, 10, 12, 14, 16, 17, 19, 21, 23, or 25.
4. The expression system of statement 1, 2, or 3, wherein the at least one promoter or the first promoter, is heterologous to the nucleic acid segment encoding the modified COI1 protein.
5. The expression system of statement 1, 2, 3, or 4, wherein the at least one promoter or the second promoter is heterologous to the nucleic acid segment encoding the modified Tlp1 protein.
6. The expression system of statement 1-4 or 5, wherein the at least one promoter, the first promoter, or the second promoter is a constitutive promoter.
7. The expression system of statement 1-5, or 6, wherein the at least one promoter, the first promoter, or the second promoter is an inducible promoter.
8. The expression system of statement 1-6, or 7, wherein the at least one promoter, the first promoter, or the second promoter is a CaMV 35S promoter, a CaMV 19S promoter, a nos promoter, an Adh1 promoter, a sucrose synthase promoter, an α-tubulin promoter, a ubiquitin promoter, an actin promoter, a cab promoter, a PEPCase promoter, an R gene complex promoter, a xylem-specific promoter, a cauliflower mosaic virus promoter, a Z10 promoter (e.g., from a 10 kD zein protein), a Z27 promoter (e.g., from a gene encoding a 27 kD zein protein, a light inducible promoter (e.g., derived from the pea rbcS gene), a seed specific promoter.
9. The expression system of statement 1-7, or 8, wherein the at least one promoter, the first promoter, or the second promoter is a rice actin1 (Act1) promoter, a CaMV 35S promoter, or a phaselin promoter.
10. A plant comprising an expression system comprising an expression cassette comprising at least one promoter operably linked to a nucleic acid that encodes a COI1 protein, a Tlp1 protein, or a combination thereof.
11. The plant of statement 10, comprising two expression cassettes, a first expression cassette comprising a first promoter operably linked to a nucleic acid that encodes a COI1 protein, and a second promoter operably linked to a nucleic acid that encodes a Tlp1 protein.
12. The plant of statement 10 or 11, wherein the COI1 protein has a sequence with at least 95% sequence identity to SEQ ID NO:1, 3, 5, 6, 8, 10, 12, 14, 16, 17, 19, 21, 23, or 25.
13. The plant of statement 10, 11, or 12, wherein the at least one promoter or the first promoter, is heterologous to the nucleic acid segment encoding the modified COI1 protein.
14. The plant of statement 10-12 or 13, wherein the at least one promoter or the second promoter is heterologous to the nucleic acid segment encoding the modified Tlp1 protein.
15. The plant of statement 10-13 or 14, wherein the at least one promoter, the first promoter, or the second promoter is a constitutive promoter.
16. The plant of statement 10-14 or 15, wherein the at least one promoter, the first promoter, or the second promoter is an inducible promoter.
17. The plant of statement 10,-15 or 16, wherein the at least one promoter, the first promoter, or the second promoter is a CaMV 35S promoter, a CaMV 19S promoter, a nos promoter, an Adh1 promoter, a sucrose synthase promoter, an α-tubulin promoter, a ubiquitin promoter, an actin promoter, a cab promoter, a PEPCase promoter, an R gene complex promoter, a xylem-specific promoter, a cauliflower mosaic virus promoter, a Z10 promoter (e.g., from a 10 kD zein protein), a Z27 promoter (e.g., from a gene encoding a 27 kD zein protein, a light inducible promoter (e.g., derived from the pea rbcS gene), a seed specific promoter.
18. The plant of statement 10-16 or 17, wherein the at least one promoter, the first promoter, or the second promoter is a rice actin1 (Act1) promoter, a CaMV 35S promoter, or a phaselin promoter.
19. The plant of statement 10-17 or 18, which is at least 2-fold, at-least 3-fold, at least 4-fold, at least 5-fold, at least 6-fold more resistant to Fusarium graminearum infection than a wild type or parental plant line that does not have the expression system.
20. The plant of statement 10-18 or 19, which is at least 2-fold, at-least 3-fold, at least 4-fold, at least 5-fold, at least 6-fold more resistant to Fusarium head blight (FHB) than a wild type or parental plant line that does not have the expression system.
21. The plant of statement 10-19 or 20, which has a mass greater than or the same as a wild type or parental plant line that does not have the expression system.
22. The plant of statement 10-20 or 21, which is a wheat, rye, maize, millet, red wild einkorn, amaranth, bulgur, farro, maize, oats, rice, sorghum, spelt, barley, alfalfa, barley, canola, cassava, cocoa, corn, grain, legume, grass, jatropa, maize, miscanthus, nut, nut sedge, oats, oilseeds, peanut, rapeseed, rice, sorghum, soybean, sugarcane, sunflower, or switchgrass plant.
23. The plant of statement 10-21 or 22, which is a grain producing species.
24. The plant of statement 10-22 or 23, which is a wheat plant.
25. A plant cell or plant seed comprising an expression system comprising an expression cassette comprising at least one promoter operably linked to a nucleic acid segment that encodes a COI1 protein, a Tlp1 protein, or a combination thereof.
26. The plant cell or plant seed of statement 25, comprising two expression cassettes, a first expression cassette comprising a first promoter operably linked to a nucleic acid that encodes a COI1 protein, and a second promoter operably linked to a nucleic acid that encodes a Tlp1 protein.
27. The plant cell or plant seed of statement 25 or 26, wherein the COI1 protein has a sequence with at least 95% sequence identity to SEQ ID NO:1, 3, 5, 6, 8, 10, 12, 14, 16, 17, 19, 21, 23, or 25.
28. The plant cell or plant seed of statement 25, 26, or 27, wherein the at least one promoter or the first promoter, is heterologous to the nucleic acid segment encoding the modified COI1 protein.
29. The plant cell or plant seed of statement 25-27 or 28, wherein the at least one promoter or the second promoter is heterologous to the nucleic acid segment encoding the modified Tlp1 protein.
30. The plant cell or plant seed of statement 25-28 or 29, wherein the at least one promoter, the first promoter, or the second promoter is a constitutive promoter.
31. The plant cell or plant seed of statement 25-29 or 30, wherein the at least one promoter, the first promoter, or the second promoter is an inducible promoter.
32. The plant cell or plant seed of statement 25-30 or 31, wherein the at least one promoter, the first promoter, or the second promoter is a CaMV 35S promoter, a CaMV 19S promoter, a nos promoter, an Adh1 promoter, a sucrose synthase promoter, an α-tubulin promoter, a ubiquitin promoter, an actin promoter, a cab promoter, a PEPCase promoter, an R gene complex promoter, a xylem-specific promoter, a cauliflower mosaic virus promoter, a Z10 promoter (e.g., from a 10 kD zein protein), a Z27 promoter (e.g., from a gene encoding a 27 kD zein protein, a light inducible promoter (e.g., derived from the pea rbcS gene), a seed specific promoter.
33. The plant cell or plant seed of statement 25-31 or 32, wherein the at least one promoter, the first promoter, or the second promoter is a rice actin1 (Act1) promoter, a CaMV 35S promoter, or a phaselin promoter.
34. The plant cell or plant seed of statement 25-32 or 33, which produces a plant that is at least 2-fold, at-least 3-fold, at least 4-fold, at least 5-fold, at least 6-fold more resistant to Fusarium graminearum infection than a wild type or parental plant line that does not have the expression system.
35. The plant cell or plant seed of statement 25-33 or 34, which produces a plant that is at least 2-fold, at-least 3-fold, at least 4-fold, at least 5-fold, at least 6-fold more resistant to Fusarium head blight (FHB) than a wild type or parental plant line that does not have the expression system.
36. The plant cell or plant seed of statement 25-34 or 35, which produces a plant that has a mass greater than or the same as a wild type or parental plant line that does not have the expression system.
37. The plant cell or plant seed of statement 25-35 or 36, which is a wheat, rye, maize, millet, red wild einkorn, amaranth, bulgur, farro, maize, oats, rice, sorghum, spelt, barley, alfalfa, barley, canola, cassava, cocoa, corn, grain, legume, grass, jatropa, maize, miscanthus, nut, nut sedge, oats, oilseeds, peanut, rapeseed, rice, sorghum, soybean, sugarcane, sunflower, or switchgrass plant cell or plant seed.
38. The plant cell or plant seed of statement 25-36 or 37, which is of a grain producing species.
39. The plant cell or plant seed of statement 25-37 or 38, which is a wheat plant cell or wheat plant seed.
40. A method comprising transforming a plant cell with the expression system of any of statements 1-9, and generating a plant therefrom.
41. A method comprising cultivating the plant of statement 1-23 or 24.
42. The method of statement 41, further comprising harvesting grain from the plant.
43. The method of statement 41 or 42 wherein the plant is part of a crop of such plants.

The specific products, compositions, and methods described herein are representative, exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

The invention illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a plant” or “a seed” or “a cell” includes a plurality of such plants, seeds or cells, and so forth. In this document, the term “or” is used to refer to a nonexclusive or, such that “A or B” includes “A but not B,” “B but not A.” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed:

1. A plant or plant seed comprising at least one heterologous expression system, each heterologous expression system comprising an expression cassette comprising at least one promoter operably linked to a nucleic acid that encodes a COI1 protein, a Tlp1 protein, or a combination thereof.

2. The plant or plant seed of claim 1, comprising two expression cassettes, a first expression cassette comprising a first promoter operably linked to a nucleic acid that encodes a COI1 protein, and a second promoter operably linked to a nucleic acid that encodes a Tlp1 protein.

3. The plant or plant seed of claim 1, wherein the COI1 protein has a sequence with at least 95% sequence identity to SEQ ID NO:1, 3, 5, 6, 8, 10, 12, 14, 16, 17, 19, 21, 23, or 25.

4. The plant or plant seed of claim 1, wherein the at least one promoter is heterologous to the nucleic acid segment encoding the COI1 protein or the Tlp1 protein.

5. The plant or plant seed of claim 1, wherein the at least one promoter is a constitutive promoter.

6. The plant or plant seed of claim 1, wherein the at least one promoter is an inducible promoter.

7. The plant or plant seed of claim 1, wherein the at least one promoter is a CaMV 35S promoter, a CaMV 19S promoter, a nos promoter, an Adh1 promoter, a sucrose synthase promoter, an α-tubulin promoter, a ubiquitin promoter, an actin promoter, a cab promoter, a PEPCase promoter, an R gene complex promoter, a xylem-specific promoter, a cauliflower mosaic virus promoter, a Z10 promoter (e.g., from a 10 kD zein protein), a Z27 promoter (e.g., from a gene encoding a 27 kD zein protein, a light inducible promoter (e.g., derived from the pea rbcS gene), a seed specific promoter.

8. The plant or plant seed of claim 1, wherein the at least one promoter is a rice actin1 (Act1) promoter, a CaMV 35S promoter, or a phaselin promoter.

9. The plant or plant seed of claim 1, comprising an expression system comprising two expression cassettes, a first expression cassette comprising a first rice actin1 (Act1) promoter operably linked to a nucleic acid that encodes a COI1 protein, and a second rice actin1 (Act1) promoter operably linked to a nucleic acid that encodes a Tlp1 protein.

10. The plant or plant seed of claim 1 that is at least 2-fold more resistant to Fusarium head blight (FHB) than a wild type or parental plant line that does not have the expression system.

11. The plant or plant seed of claim 1 that is a grain producing species.

12. The plant or plant seed of claim 1 that is a wheat plant or wheat seed.

13. A method comprising cultivating the plant or plant seed of claim 1 and harvesting grain from the plant or plant grown from the plant seed.


SEQUENCE LISTING (51 sequences)

SEQ ID NO: 1 - protein, 592 aa, Triticum aestivum
MGGEAPEPRRLSRALSLDGGGVPEEALHLVLGYVDDPRDREAASLACRRWHHIDALTRKHVTVPFCYAVSPARLLARFPRLESLGVKGKPRAAMYGLIPDDWGAYARPWVAELAAPLECLKALHLRRMVVTDDDLAALVRARGHMLQELKLDKCSGFSTDALRLVARSCRSLRTLFLEECTITDNGTEWLHDLAANNPVLVTLNFYLTYLRVEPADLELLAKNCKSLISLKISDCDLSDLIGFFQIATSLQEFAGAEISEQKYGNVKLPSKLCSFGLTFMGTNEMHIIFPFSAVLKKLDLQYSFLTTEDHCQLIAKCPNLLVLAVRNVIGDRGLGVVGDTCKKLQRLRVERGEDDPGMQEEEGGVSQVGLTAIAVGCRELENIAAYVSDITNGALESIGTFCKNLHDFRLVLLDKQETITDLPLDNGARALLRGCTKLRRFALYLRPGGLSDVGLGYIGQHSGTIQYMLLGNVGQTDGGLISFAAGCRNLRKLELRSCCFSERALALAIRQMPSLRYVWVQGYRASQTGRDLMLMARPFWNIEFTPPSTETAGRLMEDGEPCVDRQAQVLAYYSLSGKRSDYPQSVVPLYPA

SEQ ID NO: 2 - DNA, 2167 bp, Triticum aestivum
acgagcaccaccatcggagaagggccagcgggaaggggggaaatcaatccccatgcccccacccctcgccggaccagatccccggcgggccggcgcggagccttaggcggggatgggcggggaggccccggagccgcggcggctgagccgcgcgctcagcctggacggcggcggcgtcccggaggaggcgctgcacctggtgctcggctacgtggacgacccgcgcgaccgcgaggcggcctcgctggcgtgccgccgctggcaccacatcgacgcgctcacgcggaagcacgtcaccgtgcccttctgctacgccgtgtccccggcgcgcctgctcgcgcgcttcccgcgcctcgagtcgctcggggtcaagggcaagccccgcgccgccatgtacggcctcatccccgacgactggggcgcctacgcccggccctgggtcgccgagctcgccgccccgctcgagtgcctcaaggcgctccacctgcgccgcatggtcgtcaccgacgacgacctcgccgccctcgtccgcgcccgcggccacatgctgcaggagctcaagctcgacaagtgctccggcttctccaccgacgccctccgcctcgtcgcccgctcctgcagatcactgagaactttgtttctggaagaatgtacaattactgataatggcactgaatggctccatgaccttgctgccaacaatcctgttctggtgaccttgaacttctacttgacttacctcagagtggagccagctgacctcgagcttctcgccaagaattgcaagtcactaatttcgttgaagattagcgactgcgacctttcagatttgattggatttttccaaatagctacatctttgcaagaatttgctggagcggaaatcagtgagcaaaagtatggaaatgttaagcttccttcaaagctttgctccttcggacttaccttcatggggacaaatgagatgcacataatctttcctttttctgctgtactcaagaagctggatttgcagtacagttttctcaccactgaagatcattgccagctcattgcaaaatgtccaaacttactagtccttgcggtgaggaatgtgattggggatagaggactgggggttgtcggagacacatgcaagaagctacaaaggctcagagttgagcgaggggaagatgaccctggcatgcaagaagaggaaggcggagtttctcaagtaggcctaacagccatagccgtaggttgccgtgaactggaaaacatagctgcctatgtgtctgatatcacaaatggggccctggaatccatcggaacgttctgcaaaaatctccatgactttcgccttgtcctgcttgacaaacaagagacgataacagatttgccgctggacaacggtgcccgcgcgctgctcaggggctgcaccaagcttcggaggttcgctctatacctgagaccaggggggctttcagatgtaggcctcggctacatcgggcagcacagtggaaccatccagtacatgcttctgggtaacgtcgggcagacggatggtggattgatcagtttcgcagccgggtgccggaacctgcggaagcttgaactgaggagctgttgcttcagcgagcgggctctggccctcgccatacggcaaatgccttccctgaggtatgtgtgggtgcagggctacagggcctctcagaccggccgcgacctcatgctcatggcgcggcccttctggaacatcgagtttacgcctcccagcacggagaccgcgggccggctgatggaagatggggagccctgcgttgataggcaagctcaggtgctggcgtactactccctttctgggaagaggtccgactacccgcagtctgttgttcctctgtatcctgcgtgactgtaaatacattaagccggtatggtgtctctctgggacggcccctggctggccctctgcgcttctcgggcaataaggatgtttgtatgtgggtattgtatggatctggtagattttctagctgctgtgtactggaataagcgcattggtatttttgcctggtactcctatctaatcttaggaagatgtatactaaagtaacattgtgcgagtgaactgtgacactattgcgcttgcttcgcaggcataagcttgtctggtttccgcggcctgccc

SEQ ID NO: 3 - protein, 594 aa, Triticum aestivum
MGGEVPEPRRLSRALSFGVPDEALHLVMGYVDAPRDREAASLVCRRWHRIDALTRKHVTVAFCYAADPSRLLARFPRLESLALKGRPRAAMYGLISDDWGAYAAPWVARLAAPLECLKALHLRRMTVTDDDVATLIRSRGHMLQELKLDKCSGFSTDALRLVARSCRSLRTLFLEECVITDEGGEWLHELAVNNSVLVTLNFYMTELKVVPADLELLAKNCKSLLSLKISECDLSDLIGFFEAANALQDFAGGSFNEVGELTKYEKVKFPPRVCFLGLTFMGKNEMPVIFPFSASLKKLDLQYTFLTTEDHCQLISKCPNLFVLEVRNVIGDRGLEVVGDTCKKLRRLRIERGDDDPGLQEEQGGVSQLGLTAVAVGCRDLEYIAAYVSDITNGALESIGTFCKNLYDFRLVLLDRQKQVTDLPLDNGVRALLRSCTKLRRFALYLRPGGLSDIGLDYIGQYSGNIQYMLLGNVGESDHGLIRFAIGCTNLRKLELRSCCFSEQALSLAVLHMPSLRYIWVQGYKASPAGLELLLMARRFWNIEFTPPSPEGLFRMTLEGEPCVDKQAQVLAYYSLAGQRQDCPDWVTPLHPAA

SEQ ID NO: 4 - DNA, 2199 bp, Triticum aestivum
acgaggcccgcaaagcccacccccgtagcagaaagggagggagggaggaggaatctccgtctccacctccacctccatgcccccgccccccgccgggcccggcccagatctcccgcgcggcggccgctagccgatccgatccggcccgatgggcggggaggtgccggagccgcggcggctcagccgcgcgctcagcttcggcgtgcccgacgaggcgctgcacctcgtcatgggctacgtcgacgccccgcgcgaccgggaggccgcctcgctcgtctgccgccgctggcaccgcatcgacgcgctcacccgcaagcacgtcaccgtcgccttctgctacgccgccgacccctcgcgcctcctcgcccgcttcccgcgcctcgagtcgctggccctcaagggcaggccgcgcgccgccatgtacggcctcatctccgacgactggggcgcctacgccgcgccctgggtcgcacggctcgccgcgccgctcgagtgcctaaaggcgctccacctgcgacgcatgaccgtaaccgacgacgacgtcgccacgctcatccgctcccgcggccacatgctgcaggagctcaagctcgacaagtgctccggcttctccaccgacgcgctccgcctcgtcgcccgctcctgcagatccttaagaacattatttcttgaagaatgcgtgattactgacgaaggtggtgaatggcttcatgaacttgctgtcaacaattctgttcttgtgacactgaacttctacatgactgagctcaaagtggtgccggctgatctggagcttctagcaaagaactgcaaatcattactttctttaaagatcagtgagtgtgacctttcagacctgattggttttttcgaagcagccaatgcattgcaagattttgctggaggatcgttcaatgaggtaggagagctaacaaagtatgaaaaagtcaagtttccaccaagagtatgcttcttggggcttacgttcatggggaaaaatgagatgcctgttatcttccccttttctgcttcattaaagaagctggacttgcagtacactttcctcaccactgaggatcattgccagcttatctcaaaatgcccgaacctatttgttcttgaggtgaggaatgtgataggagacagagggctggaggttgtcggcgatacatgcaagaagctacgaagacttcgaattgagcgaggggatgatgatccaggtctacaagaagagcaaggaggagtttctcagttaggcctgacagcggtagctgttggttgccgagacctggagtacatagctgcctatgtatctgatatcaccaacggtgctctcgaatccatcgggaccttctgcaaaaatctctacgacttccggcttgtcctgctcgacagacaaaagcaggtaactgatctgccactcgacaacggtgttcgtgctctgttaaggagttgcaccaagctccggagatttgctctctacctgagacctggagggctctcagacataggcctcgactacatcgggcagtacagcggcaacattcagtacatgctgctgggcaacgtcggtgaatctgaccacgggttgatccgctttgcgataggatgcaccaacctgcggaagcttgagcttcggagctgctgcttcagcgagcaagccctgtccctcgcggtgctccacatgccctcgctcaggtacatatgggtgcaaggctacaaagcctctccagcaggcctcgagctcctgctcatggcgaggcgattctggaacatcgagttcacgccccccagccccgagggcttgttccgcatgacgcttgaaggagaaccctgcgtggataagcaggcccaggttcttgcctactactcccttgctgggcagaggcaggactgccctgactgggtgaccccgttgcatccagctgcatgattgattgtaaatacagtgtactacatcaagttgtgtgtacgtaggtactctaccttattgcccctcgtcccttgggcaacgatcgtgtccgaatatggtagtaatttgtatggatgtagatcattagctagctgctttggtgccctaataagctagtgctactgtagtgctgtagctgaggtgtagtgcaataagttgctgttgtcgcttgtactactatgtatgtaatcctgggaagttgtatgctaaagttgctccgtgctcgt

SEQ ID NO: 5 - protein, 583 aa, Oryza sativa
MSFGGAGSIPEEALHLVLGYVDDPRDREAVSLVCRRWHRIDALTRKHVTVPFCYAASPAHLLARFPRLESLAVKGKPRAAMYGLIPEDWGAYARPWVAELAAPLECLKALHLRRMVVTDDDLAALVRARGHMLQELKLDKCSGFSTDALRLVALSCRSLRTLFLEECSIADNGTEWLHDLAVNNPVLETLNFHMTELTVVPADLELLAKKCKSLISLKISDCDFSDLIGFFRMAASLQEFAGGAFIEQGELTKYGNVKFPSRLCSLGLTYMGTNEMPIIFPFSALLKKLDLQYTFLTTEDHCQLIAKCPNLLVLAVRNVIGDRGLGVVADTCKKLQRLRVERGDDDPGLQEEQGGVSQVGLTTVAVGCRELEYIAAYVSDITNGALESIGTFCKNLCDFRLVLLDREERITDLPLDNGVRALLRGCMKLRRFALYLRPGGLSDTGLGYIGQYSGIIQYMLLGNVGETDDGLIRFALGCENLRKLELRSCCFSEQALACAIRSMPSLRYVWVQGYKASKTGHDLMLMARPFWNIEFTPPSSENANRMREDGEPCVDSQAQILAYYSLAGKRSDCPRSVVPLYPA

SEQ ID NO: 6 - protein, 591 aa, Hordeum vulgare
MGGEAPEPRRLTRALSVDGSGVPEEALHLVFGYVDDPRDREAASLACRRWHHIDALTRKHVTVPFCYAVSPARLLARFPRLESLGVKGKPRAAMYGLISDDWGAYARPWIAELAAPLECLKALHLRRMVVTDDDLAALVLARGHMLQELKLDKCSGFSTDALRLVARSCRSLRTLFLEECTITDNGTEWLHDLAANNPVLVNLNFYLTYLRAVPADLELLARNCKSLISLKISDCDLSDLVGFFQIATSLQEFAGAEISEQMYGNVKFPSKICSFGLTFMGINEMHIIFPFSAVLKKLDLQYSFLTTEDHCQLIAKCPNLLVLAVRNVIGDRGLAVVGDTCKKLQRLRVERGEDDPGMQEEGGVSQVGLTAVAVGCRELEYIAAYVSDITNGALESIGTFCKKLYDFRLVLLDRQERITDLPLDNGARALLRGCTKLRRFALYLRPGGLSDVGLNYIGQHSGTIHYMLLGNVGQTDDGLISFAAGCRNLLKLELRSCCFSERALALAVLKMPSLRYVWVQGYRASQTGRDLMLMARPFWNIEFTPPGTESAGRLMEDGEPCVDRQAQVLAYYSLSGRRSDCPQSVVPLYPA

SEQ ID NO: 7 - DNA, 2174 bp, Hordeum vulgare
ggtggcaaaatccccatgcctcagcccctcgccggaccagatccccggcgagccagcgcgggggattaggcggggagaggcccgatcgatgggcggggaggccccggagccgcggcggctgacccgcgcgctcagcgtggacggcagcggtgtcccggaggaggcgctgcacctggtgttcgggtacgtcgacgacccgcgcgaccgggaggcggcgtcgctggcctgccgccggtggcaccacatcgacgcgctcacgcggaagcatgtcaccgtgcccttctgctacgcggtttccccggcacgcctgctcgcgcgcttcccgcgcctcgagtcgctcggggtcaagggcaagccccgcgccgccatgtacggcctcatctccgacgactggggcgcctacgctcgcccctggatagccgagctcgctgccccgctcgagtgcctcaaggcgctccacctgcgccgcatggtcgtcaccgacgacgacctcgccgcccttgtcctcgcccgcggccacatgctgcaggagctcaagctcgacaagtgctctggcttctccaccgacgccctccgcctcgtcgcccgatcctgcagatcactgagaactttgtttctggaagaatgcacaattactgataatggcactgaatggctccatgatcttgctgccaacaatcctgttctggtgaacttgaacttctacttgacttacctcagagcggtgccagctgacctcgagcttcttgccaggaattgcaagtcactaatttcattgaagatcagtgattgtgacctttcagatttagttggatttttccaaatagctacgtcattgcaagaatttgctggagcggaaattagtgagcaaatgtatggaaatgttaagtttccttcaaagatttgctcattcggacttaccttcatggggataaatgagatgcacataatctttcctttttccgctgtactcaagaagctggatttgcagtacagtttcctcaccactgaagatcattgccagctcattgcaaaatgtccaaacttactagttcttgcggtgaggaatgtgattggggatagaggattagcggttgtcggagacacatgcaagaagctacaaaggctcagagttgagagaggggaagatgatcctggtatgcaagaagaaggaggagtttctcaagtaggcctaacagccgtagccgtaggctgccgtgaactggaatacatagccgcctatgtgtctgatatcacgaacggggccctagaatctatcggaacattctgcaaaaagctttatgactttcgccttgtcctgcttgacagacaagagaggataacagatttgccactggacaatggtgcccgtgcgctgctgaggggctgcactaaacttcggaggttcgctctatacctgagaccagggggcctttcagatgtgggccttaactatattggacagcacagtggaactatccactacatgcttctgggtaacgttgggcaaacggatgacggattaatcagttttgcagctgggtgccggaacctgctgaagcttgaattaaggagctgctgcttcagcgagcgggctttggccctcgccgtactgaaaatgccttctctgaggtacgtatgggtgcagggctacagagcctctcaaactggccgcgacctcatgctcatggcaaggcccttctggaacattgagtttacgcctcccggcacggagagcgcgggtcggctgatggaagatggggagccctgtgttgataggcaagctcaggtacttgcatactactcccttagtgggaggaggtcggactgcccgcagtctgttgttcctctgtatcctgcgtgactgtacatacacaaagctggcgcatgtttgcgatggtgtagcccccgggcccttcttgggcaataaggatatgtttgtatgtgggtattgtatggatctagtagatgtctagctgctgtgtactggaataagcgcatgctatttttgcctggtactcctatctaatcctaggaagatgtatactaaagtaacaatgtgtgggtgcaactgtgacactattgtgcttgctcccaggtataagcatgcccggtttttgcaacatgttctctgttgtacataattgctctctgaaaaaaaaaatctcctggtg

SEQ ID NO: 8 - protein, 591 aa, Hordeum vulgare
MGGEAPEPRRLTRALSVDGSGVPEEALHLVFGYVDDPRDREAASLACRRWHHIDALTRKHVTVPFCYAVSPARLLARFPRLESLGVKGKPRAAMYGLISDDWGAYARPWIAELAAPLECLKALHLRRMVVTDDDLAALVLARGHMLQELKLDKCSGFSTDALRLVARSCRSLRTLFLEECTITDNGTEWLHDLAANNPVLVNLNFYLTYLRAVPADLELLARNCKSLISLKISDCDLSDLVGFFQIATSLQEFAGAEISEQMYGNVKFPSKICSFGLTFMGINEMHIIFPFSAVLKKLDLQYSFLTTEDHCQLIAKCPNLLVLAVRNVIGDRGLAVVGDTCKKLQRLRVERGEDDPGMQKEGGVSQVGLTAVAVGCRELEYIAAYVSDITNGALESIGTFCKKLYDFRLVLLDRQERITDLPLDNGARALLRGCTKLRRFALYLRPGGLSDVGLNYIGQHSGTIHYMLLGNVGQTDDGLISFAAGCRNLLKLELRSCCFSERALALAVLKMPSLRYVWVQGYRASQTGRDLMLMARPFWNIEFTPPGTESAGRLMEDGEPCVDRQAQVLAYYSLSGRRSDCPQSVVPLYPA

SEQ ID NO: 9 - DNA, 2017 bp, Hordeum vulgare
ggaggcaaaatccccatgcctcagcccctcgccggaccagatccccggcgagccagcgcgggggattaggcggggagaggcccgatcgatgggcggggaggccccggagccgcggcggctgacccgcgcgctcagcgtggacggcagcggcgtcccggaggaggcgctgcacctggtgttcgggtacgtcgacgacccgcgcgaccgggaggcggcgtcgctggcctgccgccggtggcaccacatcgacgcgctcacgcggaagcatgtcaccgtgcccttctgctacgcggtttccccggcacgcctgctcgcgcgcttcccgcgcctcgagtcgctcggggtcaagggcaagccccgcgccgccatgtacggcctcatctccgacgactggggcgcctacgctcgcccctggatagccgagctcgctgccccgctcgagtgcctcaaggcgctccacctgcgccgcatggtcgtcaccgacgacgacctcgccgcccttgtcctcgcccgcggccacatgctgcaggagctcaagctcgacaagtgctctggcttctccaccgacgccctccgcctcgtcgcccgatcctgcagatcactgagaactttgtttctggaagaatgcacaattactgataatggcactgaatggctccatgatcttgctgccaacaatcctgttctggtgaacttgaacttctacttgacttacctcagagcggtgccagctgacctcgagcttcttgccaggaattgcaagtcactaatttcattgaagatcagtgattgtgacctttcagatttagttggatttttccaaatagctacgtcattgcaagaatttgctggagcggaaattagtgagcaaatgtatggaaatgttaagtttccttcaaagatttgctcattcggacttaccttcatggggataaatgagatgcacataatctttcctttttccgctgtactcaagaagctggatttgcagtacagtttcctcaccactgaagatcattgccagctcattgcaaaatgtccaaacttactagttcttgcggtgaggaatgtgattggggatagaggattagcggttgtcggagacacatgcaagaagctacaaaggctcagagttgagagaggggaagatgatcctggtatgcaaaaagaaggaggagtttctcaagtaggcctaacagccgtagccgtaggctgccgtgaactggaatacatagccgcctatgtgtctgatatcacgaacggggccctagaatctatcggaacattctgcaaaaagctttatgactttcgccttgtcctgcttgacagacaagagaggataacagatttgccactggacaatggtgcccgtgcgctgctgaggggctgcactaaacttcggaggttcgctctatacctgagaccagggggcctttcagatgtgggccttaactatattggacagcacagtggaactatccactacatgcttctgggtaacgttgggcaaacggatgacggattaatcagttttgcagctgggtgccggaacctgctgaagcttgaattaaggagctgctgcttcagcgagcgggctttggccctcgccgtactgaaaatgccttctctgaggtacgtatgggtgcagggctacagagcctctcaaactggccgcgacctcatgctcatggcaaggcccttctggaacattgagtttacgcctcccggcacggagagcgcgggtcggctgatggaagatggggagccctgtgttgataggcaagctcaggtacttgcatactactcccttagtgggaggaggtcggactgcccgcagtctgttgttcctctgtatcctgcgtgactgtacatacacaaagctggcgcatgtttgcgatggtgtagcccccgggcccttcttgggcaataaggatatgtttgtatgtgggtattgtatggatctagtagatgtctagctgctgtgtactggaataagcgcatgctatttttgcctggt

SEQ ID NO: 10 - protein, 599 aa, Sorghum bicolor
MGGEAPEPRRLTRALSIGGGDGGWVPEEMLHLVMGFVEDPRDREAASLVCRRWHRVDALSRKHVTVPFCYAVSPARLLARFPRLESLAIKGKPRAAMYGLIPDDWGAYARPWVAELAAPLECLKALHLRRMVVTDDDLAELVRARGHMLQELKLDKCTGFSTDGLRLVARSCRSLRTLFLEECQINDKGSEWIHDLADGCPVLTTLNFHMTELQVMPADLEFLARSCKSLISLKISDCDVSDLIGFFQFATALEEFAGGTFNEQGELTMYGNVRFPSRLCSLGLTFMGTNEMPIIFPFSAILKKLDLQYTVLTTEDHCQLIAKCPNLLVLAVRNVIGDRGLGVVADTCKKLQRLRIERGDDEGGVQEEQGGVSQVGLTAIAVGCRELEYIAAYVSDITNGALESIGTFCKKLYDFRLVLLDREERITELPLDNGVRALLRGCTKLRRFALYLRPGGLSDAGLGYIGQCSGNIQYMLLGNVGETDDGLFSFALGCVNLRKLELRSCCFSERALALAILRMPSLRYVWVQGYKASQTGRDLMLMARPFWNIEFTPPSSENAGRLMEDGEPCVDSHAQILAYHSLAGKRLDCPQSVVPLYPA

SEQ ID NO: 11 - DNA, 2401 bp, Sorghum bicolor
ctcgtccgtcctcctctccactctctcttctccctccaataattctctcctctctctctgcactctgcttgctccacctccaagcaccaccgaatcagggccagtgggagcagcagcagcagcgagtgggagcagaggagggcagagaatcccatgtctccgcccctcgctagagcagatcctcggcgagccgggcgtggagctgcttcggtagaaaagcgagccaactgagcctgcgagcgcctgatccgcccgcggcccgatcgggatcgatgggcggtgaggcgccggagccccggcggctgacccgcgcgctgagcatcggcggcggcgacggcggctgggtccccgaggagatgctgcacctggtgatggggttcgtcgaggacccgcgcgaccgggaggccgcgtcgctggtgtgccgccggtggcaccgcgtcgacgcgctgtcgcggaagcacgtcacggtgcccttctgctacgccgtgtccccggcgcgcctgctcgcgcggttcccgcggctcgagtcgctggccatcaaggggaagccccgcgcggccatgtacggcctcataccggacgactggggcgcctacgcccgcccctgggtcgccgagctcgccgcgccgctcgagtgcctcaaggcgctccacctccgacgcatggtcgtcacggacgacgacctcgccgagctcgtccgtgccaggggacacatgctgcaggagctcaagctcgacaagtgcaccggcttctccacggatggactccgcctcgttgcgcgctcctgcagatcactgagaactttgtttctggaagaatgtcaaattaatgataaaggcagtgaatggatccacgatcttgcagacggttgtcctgttctgacaacattgaatttccacatgactgagcttcaagtgatgccagctgacctagagtttcttgcaaggagctgcaagtcactgatttccttgaagattagcgactgtgatgtttcagatttgatagggttcttccaatttgccacagcactggaagaatttgctggagggacattcaatgagcaaggggaactcaccatgtatgggaatgtcagatttccatcaagactatgctccttgggacttactttcatgggaacaaatgaaatgcctattatatttcctttttctgcaatactgaagaagctggatttgcagtacactgtcctcaccactgaagaccattgccagcttattgcaaaatgtccgaacttactagttctcgcggtgaggaatgtgattggagatagaggattaggagttgttgcagatacatgcaagaagctccaaaggctcagaattgagcgaggagacgatgaaggaggtgtgcaagaagagcagggaggggtctctcaagtgggcttgacggctatagccgtcggttgccgtgaactggaatacatagctgcctatgtgtctgatataaccaatggggccctggaatctatcgggacattctgcaaaaaactctatgacttccggcttgttctgcttgatagagaagagaggataacagaattgccactggacaatggtgtccgagctttgttgaggggctgcaccaaacttcggaggtttgctctgtacttgagaccaggagggctctcagatgcaggtctcggctacattggacagtgcagtggaaacatccaatacatgcttctcggtaatgttggggaaactgatgatggattgttcagtttcgcattgggatgcgtaaacctgcggaagcttgaactcaggagttgttgcttcagcgagcgagctctggccctcgccatactacgcatgccttccctgaggtacgtatgggttcagggctacaaagcgtctcaaaccggccgagacctcatgctcatggcgaggcccttctggaacatagagtttacacctcccagttccgagaacgcaggtcggttgatggaagatggggaaccttgtgtagatagtcatgctcagatactcgcataccactccctcgccggtaagaggttggactgcccacaatccgtggtccctttgtatcctgcctgagtgtaaatagactaagctggtgtttttctccctcatccctgcttccttagcctcctggtcaacaagaacgatgttgatgacttgatatgtggttattgtatggatctagatggctagctgctacgtactgtaataagctactagtagctgagatgtcctggaataagcccttgctattttcgcctgtactgctatctaatcctaggaagatgtatactactaagtaacggtggaagatgtgagtcttgcttgctcgccctgatttgtactattggaggtataagaatacctgggtttttgccgcctactttgagcattgagatgtgtct

SEQ ID NO: 12 - protein, 592 aa, Arabidopsis thaliana
MEDPDIKRCKLSCVATVDDVIEQVMTYITDPKDRDSASLVCRRWFKIDSETREHVTMALCYTATPDRLSRRFPNLRSLKLKGKPRAAMFNLIPENWGGYVTPWVTEISNNLRQLKSVHFRRMIVSDLDLDRLAKARADDLETLKLDKCSGFTTDGLLSIVTHCRKIKTLLMEESSFSEKDGKWLHELAQHNTSLEVLNFYMTEFAKISPKDLETIARNCRSLVSVKVGDFEILELVGFFKAAANLEEFCGGSLNEDIGMPEKYMNLVFPRKLCRLGLSYMGPNEMPILFPFAAQIRKLDLLYALLETEDHCTLIQKCPNLEVLETRNVIGDRGLEVLAQYCKQLKRLRIERGADEQGMEDEEGLVSQRGLIALAQGCQELEYMAVYVSDITNESLESIGTYLKNLCDFRLVLLDREERITDLPLDNGVRSLLIGCKKLRRFAFYLRQGGLTDLGLSYIGQYSPNVRWMLLGYVGESDEGLMEFSRGCPNLQKLEMRGCCFSERAIAAAVTKLPSLRYLWVQGYRASMTGQDLMQMARPYWNIELIPSRRVPEVNQQGEIREMEHPAHILAYYSLAGQRTDCPTTVRVLKEPI

SEQ ID NO: 13 - DNA, 2512 bp, Arabidopsis thaliana
gcaaaaatgaaaagaaaaacatagaagtagagagaagatcgcatctcgaccgtcaacttcagtgtatgaaataatgatcgtcccacttgatcctcaaaaatattattaaccaaacaaaatttgattccatcgtcccactttcttcttcttcctcccaatccgcctcttcttcctacgcgtgtcttcttctccctcactctctcaatctctagtcttctccgattcaccggatctttcctttcttacttctttcttctcactctggtggttatgtgtggatctgcgacctcgatttcaattcgaagtcgtcggtttcttctctaaatcgaatctttccaggattcgtttgtttttttcttttgtttttttttcgatccgatggaggatcctgatatcaagaggtgtaaattgagctgcgtcgcgacggttgatgatgtcatcgagcaagtcatgacctatataactgacccgaaagatcgcgattcggcttctttggtgtgtcggagatggttcaagattgattccgagacgagagagcatgtgactatggcgctttgctacactgcgacgcctgatcgtcttagccgtcgattcccgaacttgaggtcgctcaagcttaaaggcaagcctagagcagctatgtttaatctgatccctgagaactggggaggttatgttactccttgggttactgagatttctaacaaccttaggcagctcaaatcggtgcacttccgacggatgattgtcagtgacttagatctagatcgtttagctaaagctagagcagatgatcttgagactttgaagctagacaagtgttctggttttactactgatggacttttgagcatcgttacacactgcaggaaaataaaaactttgttaatggaagagagttcttttagtgaaaaggatggtaagtggcttcatgagcttgctcagcacaacacatctcttgaggttttaaacttctacatgacggagtttgccaaaatcagtcccaaagacttggaaaccatagctagaaattgccgctctctggtatctgtgaaggtcggtgactttgagattttggaactagttgggttctttaaggctgcagctaatcttgaagaattttgtggtggctccttgaatgaggatattggaatgcctgagaagtacatgaatctggtttttccccgaaaactatgtcggcttggtctctcttacatgggacctaatgaaatgccaatactatttccattcgcggcccaaatccgaaagctggatttgctttatgcattgctagaaactgaagaccattgtacgcttatccaaaagtgtcctaatttggaagttctcgagacaaggaatgtaatcggagataggggtctagaggtccttgcacagtactgtaagcagttgaagcggctgaggattgaacgcggtgcagatgaacaaggaatggaggacgaagaaggcttagtctcacaaagaggattaatcgctttggctcagggctgccaggagctagaatacatggcggtgtatgtctcagatataactaacgaatctcttgaaagcataggcacatatctgaaaaacctctgtgacttccgccttgtcttactcgaccgggaagaaaggattacagatctgccactggacaacggagtccgatctcttttgattggatgcaagaaactcagacgatttgcattctatctgagacaaggcggcttaaccgacttgggcttaagctacatcggacagtacagtccaaacgtgagatggatgctgctgggttacgtaggtgaatcagatgaaggtttaatggaattctcaagaggctgtccaaatctacagaagctagagatgagaggttgttgcttcagtgagcgagcaatcgctgcagcggttacaaaattgccttcactgagatacttgtgggtacaaggttacagagcatcgatgacgggacaagatctaatgcagatggctagaccgtactggaacatcgagctgattccatcaagaagagtcccggaagtgaatcaacaaggagagataagagagatggagcatccggctcatatattggcttactactctctggctggccagagaacagattgtccaacaactgttagagtcctgaaggagccaatatgatatgacccaaaaaacaggtttgtatataaagatttttagtctcgagttttggggtttccacaaactgtgtactatactactttggttctttttttgtttcatgttgtgtcgtcgatgtttttgggagattacatagagtcagtcttgtttgttgtatggtcattacttctttatttttcctcaggggtctgtttactttaatttctttaataaaaccccgaagattttgagagatttctttatcgtccatggtgttgacttctgagagctatatttgtttggattggcatctgaaactttatttgtggttgtgattgttttgataacattagtaaaaaggcaaataatagagtac

SEQ ID NO: 14 - protein, 590 aa, Brassica rapa
MEDPDIKKCRLSSVTVDDVIEQVMPYITDPKDRDSASLVCRRWFEIDSETREHVTMALCYTSTPDRLSRRFPNLRSIKLKGKPRAAMFNLIPENWGGFVTPWVNEIASSLRRLKSVHFRRMIVSDLDLDVLAKARLDELEALKLDKCSGFSTDGLFSIVKHCRKMKTLLMEESSFVEKDGNWLHELALHNTSLEVLNFYMTEFAKINAKDLESIARNCRSLVSVKIGDFEMLELVGFFKAATNLEEFCGGSLNEEIGRPEKYMNLTFPPKLCCLGLSYMGPNEMPILFPFAAQIRKLDLIYALLATEDHCTLIQKCPNLEVLETRNVIGDRGLEVLGQCCKKLKRLRIERGEDEQGMEDEEGLVSQRGLVALAQGCQELEYMAVYVSDITNESLESIGTYLKNLCDFRLVLLDQEERITDLPLDNGVRSLLIGCKKLRRFAFYLRQGGLTDVGLSYIGQYSPNVRWMLLGYVGESDEGLMEFSRGCPKLQKLEMRGCCFSERAIAAAVLKIPSLRYLWVQGYRASTTGQDLRLMSRPYWNIELIPARKVPEVNQLGEVREMEHPAHILAYYSLAGERTDCPPTVKVLREA

SEQ ID NO: 15 - DNA, 2157 bp, Brassica rapa
gccacttcttcctcctctcctcacgctccacgtcccctgctagcatccctcccgcttcctcctccgatctctgctcgtcttatcttcactctctactgtattactttggatctgcgagagattcgtgtaattgaaatcgatctcgtccctcagctggtattcgaatttgttgattgttttggtttgttttagattcgatttcgatttgttacatggaggatccggatatcaagaagtgcagattgagctccgtgacggtcgatgacgtcatcgagcaggtcatgccttacataaccgatccgaaagatcgagactccgcttccctcgtgtgccggaggtggttcgagatcgactccgagacgagggagcacgtgaccatggccttgtgctacacctcgacgcccgatcgtctcagccgtaggtttcccaatctgaggtcgatcaagctcaaagggaagccgagagcagctatgttcaatctcatccccgagaactggggagggtttgttaccccttgggtcaacgagatagcttcgtcgctgcgaaggctcaagtctgtgcattttaggcggatgattgtgagcgatttggatctggatgttttggctaaggcgaggttggatgagctcgaggcgttgaagcttgataagtgctcgggtttctctacggatggacttttcagcatcgttaagcactgcaggaaaatgaaaacattgttaatggaagagagttcttttgttgaaaaggatggtaactggcttcatgaacttgctctgcacaacacttctctcgaggttctaaatttctacatgactgagtttgcaaaaatcaatgccaaagacttggaaagcatagctagaaattgccgctctctggtttctgtgaagatcggtgactttgagatgttggaactagtcgggttctttaaagctgcaactaatcttgaagaattttgtggtggctccttaaatgaagaaattggaagaccggagaagtatatgaatctgactttccctccaaaactatgttgtctgggcctttcttacatgggacctaatgaaatgccaatactgtttccattcgctgcccaaatccggaagctggatctgatctatgcattgctcgcaactgaggatcattgtacacttattcaaaagtgtcctaatttggaagttctcgagacaaggaatgtaattggagataggggtctagaggttcttggacagtgctgtaagaagttgaagcggctgaggattgaacggggtgaagatgaacaaggaatggaggatgaagaaggcttagtctcacaaagaggattagtcgctttggctcagggctgccaggagctagaatacatggcggtgtatgtctcagatataaccaacgagtctctcgaaagcataggcacatatctgaaaaacctctgtgacttccgcctcgtcttactcgaccaagaagagagaataacagatctgccactggacaacggagtcagatccctcttgatcggatgcaaaaaactcagacggtttgcattctatctcagacaaggcggcttaacagacgtggggttaagctacatcggacagtacagtccaaacgtgaggtggatgcttctcggttacgttggtgaatcagacgaaggcctaatggaattctcaagaggatgtccgaaactacagaagctggagatgagaggttgttgcttcagcgagcgagcaatagctgcagcggtactgaaaatcccttcgctgagatacctgtgggtacaaggctatagagcatcgacgacgggacaagacctgaggctaatgtctagaccgtactggaacatcgagctgattccggcaagaaaagtcccggaagtgaatcagcttggagaggtgagagagatggagcatcctgctcatatactggcttactactctctggctggtgagagaacagattgtccaccaacggttaaagtcctgagggaggcatgatgatgatgatgaaaagcaggtttgtacataaagatttggttttgaggtttccacgaactgtcgaatggattctattttttctttattggtgtattgtctgtagttttgagagattccataaagacttttgagagattgaaataagaagagagaaaactagtctttcagaaga

SEQ ID NO: 16 - protein, 703 aa, Brassica napus
MLQRIFWMFFFSFNMLTRYFIKTPPGYFCRLARCAAYATRLTKQTDSIASSPPSIYIKNNNYPLCPLDPKLLLLLSTLLIPSFTHTYATSSSSPHAPQIRVIEIDLIRFRFVTMEDPDIKKCRLSSVTVDDVIEQVMPYITDPKDRDSASLVCRRWFEIDSETREHVTMALCYTSTPDRLSRRFPNLRSIKLKGKPRAAMFNLIPENWGGFVTPWVNEIASSLRRLKSVHFRRMIVSDLDLDVLAKARLDELEALKLDKCSGFSTDGLFSIVKHCRKMKTLLMEESSFVEKDGNWLHELALHNTSLEVLNFYMTEFAKINAKDLESIARNCRSLVSVKIGDFEMLELVGFFKAATNLEEFCGGSLNEEIGRPEKYMNLTFPPKLCCLGLSYMGPNEMPILFPFAAQIRKLDLIYALLATEDHCTLIQKCPNLEVLETRNVIGDRGLEVLGQCCKKLKRLRIERGEDEQGMEDEEGLVSQRGLVALAQGCQELEYMAVYVSDITNESLESIGTYLKNLCDFRLVLLDQEERITDLPLDNGVRSLLIGCKKLRRFAFYLRQSGLTDVGLSYIGQYSPNVRWMLLGYVGESDEGLMEFSRGCPKLQKLEMRGCCFSERAIAAAVLKIPSLRYLWVQGYRASTTGQDLRLMSRPYWNIELIPARKVPEVNQLGEVREMEHPAHILAYYSLAGERTDCPPTVKVLREA

SEQ ID NO: 17 - protein, 592 aa, Brassica oleracea
MTMEDPDIKKCRLSSVTVDDVIEQVMPYITDPKDRDSASLVCRRWFEIDSETREHVTMALCYTSTPDRLSRRFPNLRSIKLKGKPRAAMFNLIPENWGGFVTPWVNEVASSLPRLKSVHFRRMIVSDLDLDVLAKARLDELEALKLDKCSGFSTDGLFSIVKHCRKMKTLLMEESSFVEKDGNWLHELALHNTSLEVLNFYMTEFAKINAKDLESIARNCRSLVSVKIGDFEMLELVGFFKAATNLEEFCGGSFNEEIGRPEKYMNLTFPPKLCCLGLSYMGPNEMPILFPFAAQIRKLDLIYALLATEDHCTLIQKCPNLEVLETRNVIGDRGLEVLGQCCKKLKRLRIERGEDEQGMEDEEGLVSQRGLVALAQGCQELEYMAVYVSDITNESLESIGTYLKNLCDFRLVLLDQEERITDLPLDNGVRSLLIGCKKLRRFAFYLRQGGLTDVGLSYIGQYSPNVRWMLLGYVGESDEGLMEFSRGCPKLQKLEMRGCCFSERAIAAAVLKIPSLRYLWVQGYRASTTGQDLRLMSRPYWNIELIPARKVPEVNQLGEVREMEHPAHILAYYSLAGERTDCPPTVKVLREA

SEQ ID NO: 18 - DNA, 2084 bp, Brassica oleracea
attattattatcaacacttttgattccttcctccacacacactcacgccacttcttcctcctctcctcacgctccacctatcgtgattcctatactcgatttcgatttgttatccgtttgtttgatgacgatggaggatccggatatcaagaagtgcagattgagctccgtgacggtcgatgacgtcatcgagcaggtcatgccttacataaccgatccgaaagatcgagactccgcttccctcgtgtgccggaggtggttcgagatcgactccgagacgagggagcacgtgaccatggcactatgctacacctcgactcctgaccgtctcagccgtaggtttccgaatctgaggtcgattaagctcaaagggaagccgagagcagctatgttcaatctcatccccgagaactggggagggtttgttaccccttgggtcaacgaggtagcttcatctctgccaaggctcaagtctgtgcattttaggcggatgattgtcagcgatttggatcttgatgttttggctaaggcgaggttggatgagctcgaggcgttgaagctcgataagtgctcaggtttctctacggatggacttttcagcatcgttaagcactgcaggaaaatgaaaacattgttaatggaagagagttcttttgttgaaaaggatggtaactggctgcatgaacttgctctgcacaacacttctcttgaggttctaaatttctacatgactgagtttgcaaaaatcaatgccaaagacttggaaagcatagctagaaattgccgctctctggtttctgtgaagatcggtgactttgagatgttggaactagtcgggttctttaaagctgcaactaatcttgaagaattttgtggcggctccttcaatgaagaaattggaagaccggagaagtatatgaatctgactttccctccaaaactatgttgtcttggcctttcttacatgggacctaatgaaatgccaatactgtttccattcgctgcccaaatccggaagctggatctgatctatgcattgctcgcaactgaggatcattgtacacttattcaaaagtgtcctaatttggaagttctcgagacaaggaatgtaattggagataggggtctagaggttcttggacagtgctgtaagaagttgaagcggctgaggattgaacggggtgaagatgaacaaggaatggaggatgaagaaggcctagtatcacaaagaggattagtcgctttggctcagggctgccaggagctagaatacatggcggtgtatgtctcagatataaccaacgagtctctcgaaagcataggcacatatctgaaaaacctctgtgacttccgcctcgtcttactcgaccaagaagagagaataacagatctgccactagacaacggagtccgatccctcttgatcggatgcaagaaactcagacggtttgcattctatctcagacaaggcggcttaacagacgtggggttaagctacatcggacagtacagtccaaacgtgaggtggatgcttctcggttacgttggtgaatcagacgaaggcctaatggagttctcaagaggatgtccgaaactacagaagctggagatgagaggttgttgcttcagcgagcgagcaatagctgcagcggtactgaaaatcccttcgctgagatacctgtgggtacaaggctacagagcatcaacgacgggacaagacctgaggctaatgtctagaccgtactggaacatcgagctgattccggcaagaaaagtcccagaagtgaatcagcttggagaggtgagagagatggagcatcctgctcatatactggcttactactctctggctggtgagagaacagattgtccaccaactgttaaagtcctgagggaggcatgatgatgatgatgatgatgatgaaaagcaggtttgtacataaagatttggttttgaggtttccacgaactgtcgaatggattctatttttctttattggtgtattgtctgtagttttgagagattccataaagacttttgagagattgaaataagaagagagaaaactagtctattcagaaga

SEQ ID NO: 19 - protein, 593 aa, Theobroma cacao
MEENDNKMNKTMTSPVGMSDVVLGCVMPYIHDPKDRDAVSLVCRRWYELDALTRKHITIALCYTTSPDRLRRRFQHLESLKLKGKPRAAMFNLIPEDWGGYVTPWVNEIAENFNCLKSLHFRRMIVKDSDLEVLARSRGKVLQVLKLDKCSGFSTDGLLHVGRSCRQLKTLFLEESLIVEKDGQWLHELAVNNSVMETLNFYMTDLVKVSFEDLELIARNCRNLASVKISDCEILDLVGFFPAAAVLEEFCGGSFNEQPDRYHAVSFPPKLCRLGLTYMGKNEMPIVFPFASLLKKLDLLYALLDTEDHCLLIQRCPNLEVLETRNVIGDRGLEVLARSCKRLKRLRIERGADEQGMEDEEGVVSQRGLMALAQGCLELEYLAVYVSDITNASLEYIGTYSKNLSDFRLVLLDREERITDLPLDNGVRALLRGCEKLRRFALYLRPGGLTDVGLSYIGQYSPNVRWMLLGYVGESDAGLLEFSKGCPSLQKLEMRGCCFSEHALAVTVMQLTSLRYLWVQGYRASQSGRDLLAMARPFWNIELIPARRVVMNDQVGEAVVVEHPAHILAYYSLAGPRTDFPETVIPLDPLVAA

SEQ ID NO: 20 - DNA, 2333 bp, Theobroma cacao
aagtttcagctctccttctctgtttcacgtttctgtgggcgctctctactctgccatgccttctctacacgacccatttttgacccgattcgtttagccccgggggaaatttgcttcgtttcagatcctaccgccgtttcgtttcttccacttccgtaaaagagaagagttccacgcccgtttcttcttcttcttcttcttcagatcagtctttttttttttttgccgtttcgcgtttctggtttatttgggctgaaaagatccgattcgattgtattgaatggaggaaaatgataacaagatgaacaaaacgatgacgtcaccagtcggtatgtcggacgtcgttttaggctgcgtgatgccgtacatccacgacccgaaagaccgggacgcagtttcgctcgtgtgccgacgttggtacgagctcgacgcgttgacgaggaagcacataacgatcgcgctttgctacacgacgagtcccgatcggttgcgacgtcgtttccagcacttggaatctttgaagttgaaaggcaagcctcgggcggcgatgttcaatttgatacctgaggattggggagggtacgtgacgccgtgggtgaatgagatagctgagaattttaattgcttgaaatctttgcattttagaaggatgattgttaaagattcggatctggaagttttggctcggtctagagggaaggttttgcaggttttgaagcttgataaatgctctggtttctctactgatggtctcttgcacgttggacgctcctgccggcaattaaaaaccttgttcctggaagagagcttaattgttgagaaagatggtcaatggcttcatgagcttgcagtaaataactcagttatggagactttgaacttttatatgacagatcttgtcaaagtgagttttgaagaccttgaacttattgctagaaattgtcgcaacttggcctctgtgaaaattagcgattgtgaaattttggatcttgttggtttctttcctgctgctgctgttttagaagaattttgtggtggttctttcaatgagcaaccggataggtaccatgctgtatcattccccccaaagttatgccgtttgggtttaacatacatggggaagaatgaaatgccaattgtgttcccttttgcatccttgcttaaaaagttggatctcctctatgcattacttgacacagaagaccactgcttgttaattcagagatgccccaacttagaagttcttgagacaaggaatgttattggagatagaggattagaagttcttgctcgaagttgtaagagactaaagaggcttagaattgaaaggggtgctgatgagcagggaatggaggatgaagaaggtgtggtttcacaaagaggattaatggctttagctcagggatgccttgaattggaatacttggctgtttatgtatctgacatcaccaatgcatcattggaatacattgggacttactcaaaaaatctctctgattttcgcctagtcttgcttgaccgagaagaaaggataacagatttgcctcttgataatggagtccgggctctattgaggggctgtgaaaagcttagaagatttgctctgtacctccgacctggtggtttgactgatgtaggcctcagttatattgggcaatacagtccgaatgtaagatggatgcttctaggttatgttggggagtcggatgccgggcttttggagttctctaagggatgcccaagcctgcagaaactagaaatgaggggttgttgcttcagtgagcatgcacttgcagttactgtgatgcaattaacttccttgaggtatttgtgggtgcaaggatatagagcgtcacaatcaggtcgtgatcttttagcaatggctcgtccattttggaatatcgagctaattcctgcaagacgagtagttatgaatgatcaggttggagaggctgttgtggttgagcatccggctcatatactcgcgtattactccctagctggaccaagaacagattttccagaaactgttattcctttggatccattagttgctgcgtagagctgtaaatatgacctatttttcgaagtgtccatttttcccatccacgttctgtctataaagtttctgcacctttctcttttctcttttcctttcctttttgtttagagggtttccaatttgatatttcattttcgattttatttctagactttgtcctgtaataagattgtgttttcttctgtaattttgaaagcacttgcactcttggtgggctactgtttttgtcccttgtccctggaaaaagtagtgaatgactcttaacggaata

SEQ ID NO: 21 - protein, 590 aa, Glycine max
MTEDRNVRKTRVVDLVLDCVIPYIDDPKDRDAVSQVCRRWYELDSLTRKHVTIALCYTTTPARLRRRFPHLESLKLKGKPRAAMFNLIPEDWGGHVTPWVKEISQYFDCLKSLHFRRMIVKDSDLRNLARDRGHVLHSLKLDKCSGFTTDGLFHIGRFCKSLRVLFLEESSIVEKDGEWLHELALNNTVLETLNFYLTDIAVVKIQDLELLAKNCPNLVSVKLTDSEILDLVNFFKHASALEEFCGGTYNEEPEKYSAISLPAKLCRLGLTYIGKNELPIVFMFAAVLKKLDLLYAMLDTEDHCMLIQKCPNLEVLETRNVIGDRGLEVLGRCCKRLKRLRIERGDDDQGMEDEEGTVSHRGLIALSQGCSELEYMAVYVSDITNASLEHIGTHLKNLCDFRLVLLDHEEKITDLPLDNGVRALLRGCNKLRRFALYLRRGGLTDVGLGYIGQYSPNVRWMLLGYVGESDAGLLEFSKGCPSLQKLEMRGCSFFSERALAVAATQLTSLRYLWVQGYGVSPSGRDLLAMARPFWNIELIPSRKVAMNTNSDETVVVEHPAHILAYYSLAGQRSDFPDTVVPLDTATCVDT

SEQ ID NO: 22 - DNA, 1773 bp, Glycine max
atgacggaggatcggaacgtgcggaagacacgtgtggtcgacctggtcctcgactgtgtcatcccttacatcgacgaccccaaggaccgcgacgccgtctcacaggtctgccgacgctggtacgaactcgactccctcactcggaagcacgtcaccatcgcgctctgctacaccaccacgccggcgcgcctccgccgccgcttcccgcaccttgagtcgctcaagctcaagggcaagccccgagcagcaatgttcaacttgatacccgaggattggggaggccacgtcaccccatgggtcaaggagatttctcagtacttcgattgcctcaagagtctccacttccgccgtatgattgtcaaagattccgatcttcggaatctcgctcgtgaccgcggccacgtgcttcactctctcaagcttgacaagtgctccggtttcaccaccgatggtcttttccatatcggtcgcttttgcaagagtttaagagtcttgtttttggaggaaagctcaattgttgagaaggacggagaatggttacacgagcttgctttgaataatacagttcttgagactctcaatttttacttgacagatattgctgttgtgaagattcaggaccttgaacttttagctaaaaattgccccaacttagtgtctgtgaaacttactgacagtgaaatactggatcttgtgaacttctttaagcatgcctctgcactggaagagttttgtggaggcacctacaatgaagaaccagaaaaatactctgctatatcattaccagcaaagttatgtcgattgggtttaacatatattggaaagaatgagttgcccatagtgttcatgtttgcagccgtactaaaaaaattggatctcctctatgcaatgctagacacggaggatcattgcatgttaatccaaaagtgtccaaatctggaagtccttgagacaaggaatgtaattggagacagagggttagaggttcttggtcgttgttgtaagaggctaaaaaggcttaggattgaaaggggtgatgatgatcaaggaatggaggatgaagaaggtactgtgtcccatagagggctaatagccttgtcacagggctgttcagagcttgaatacatggctgtttatgtgtctgatattacaaatgcatctctggaacatatcggaactcacttgaagaacctctgcgattttcgccttgtgttgcttgaccacgaagagaaaataactgatttgccacttgacaatggggtgagggctctactgaggggctgtaacaagctgaggagatttgctctatatctcaggcgtggcgggttgaccgatgtaggtcttggttacattggacagtacagtccaaatgtgagatggatgctgcttggttatgtgggggagtctgatgcagggcttttggaattctctaaagggtgtcctagtcttcagaaactagaaatgagagggtgttcatttttcagtgaacgtgcacttgctgtggctgcaacacaattgacttctcttaggtacttgtgggtgcaagggtatggtgtatctccatctggacgtgatcttttggcaatggctcgccccttttggaacattgagttaattccttctagaaaggtggctatgaataccaattcagatgagacggtagttgttgagcatcctgctcatattcttgcatattattctcttgcagggcagagatcagattttccagatactgttgtgcctttggacactgccacatgcgttgacacctag

SEQ ID NO: 23 - protein, 599 aa, Zea mays
MGGEAPEPRRLTRALSIGGGDGGWVPEEMLQLVMGFVEDPRDREAASLVCHRWHRVDALSRKHVTVPFCYAVSPARLLARFPRLESLAVKGKPRAAMYGLIPDDWGAYARPWITELAAPLECLKALHLRRMVVTDDDLAELVRARGHMLQELKLDKCTGFSTHGLRLVARSCRSLRTLFLEECQIDDKGSEWIHDLAVCCPVLTTLNFHMTELEVMPADLKLLAKSCKSLISLKISDCDLSDLIEFFQFATALEEFAGGTFNEQGELSKYVNVKFPSRLCSLGLTYMGTNEMPIMFPFSAILKKLDLQYTFLTTEDHCQLIAKCPNLLVLAVRNVIGDRGLGVVADTCKKLQRLRIERGDDEGGVQEEQGGVSQVGLTAIAVGCRELEYIAAYVSDITNGALESIGTFCKKLYDFRLVLLDREERITDLPLDNGVRALLRGCTKLRRFALYLRPGGLSDAGLGYIGQCSGNIQYMLLGNVGETDDGLISFALGCVNLRKLELRSCCFSERALALAILHMPSLRYVWVQGYKASQTGRDLMLMARPFWNIEFTPPNPKNGGWLMEDGEPCVDSHAQILAYHSLAGKRLDCPQSVVPLYPA

SEQ ID NO: 24 - DNA, 2169 bp, Zea mays
acccctgcttgctgcagcttcaagcactaccgaatcagggcgagtgggagcagaggagggaatcccatgtctccgcccctcgctggagcagatcgttgtcgagccgacgtggagctgctgcggtagaaagctagcggagcctgcgagctagcctgatccgtccgcagtccgatcgggatcgatgggtggggaggcgccggagccgcggcggctgacccgggcgctgagcatcggcggcggtgacggcggctgggttcccgaggagatgctgcaactcgtgatggggttcgtcgaggacccgcgcgaccgggaggccgcgtcgctggtgtgtcaccggtggcaccgcgtcgacgcgctctcgcggaagcacgtgacggtgcccttctgctacgccgtttccccggcacgcctgctcgcgcggttcccgcggctcgagtcgctcgcggtgaaggggaagccccgcgcggccatgtacgggctcatacccgacgactggggcgcctacgcccgcccgtggatcaccgagctcgccgcgccgctcgagtgcctcaaggcgctccacctccgacgcatggtcgtcacagacgacgacctcgccgagctcgtccgtgccagggggcacatgctgcaggagctgaagctcgataagtgcaccggcttctccactcatggactccgcctcgttgcccgctcctgcagatcactgaggactttatttttggaagaatgtcaaattgatgataagggcagtgaatggatccacgatctcgcagtctgctgtcctgttctgacaacattgaatttccacatgactgagcttgaagtgatgccagctgacctaaagcttcttgcaaagagctgcaagtcactgatttcattgaagattagtgactgcgatctttcagatttgatagagttcttccaatttgccacagcactggaagaatttgctggagggacattcaatgagcaaggggaactcagcaagtatgtgaatgttaaatttccatcaagactatgctccttgggacttacttacatgggaacaaatgaaatgcccattatgttccctttttctgcaatactaaagaagctggatttgcaatacactttcctcaccactgaggaccattgccagctcattgcaaaatgcccgaacttactagttctcgcggtgaggaatgtgattggagatagaggattaggagttgttgcggatacgtgcaagaagctccaaaggctcagaatagagcgaggagatgatgaaggaggtgtgcaagaagagcagggaggggtctctcaagtgggcttgacggctatagccgtaggttgccgtgagctggaatatatagctgcctatgtgtctgatataaccaatggggccttggaatctatcgggacattctgcaaaaaactatacgacttccggcttgttctacttgatagagaagagaggataacagacttgccactggacaatggtgtccgagctttgttgaggggctgcaccaagcttcggaggtttgctctgtacttgagaccaggagggctctcagatgcaggtctcggctacattggacagtgcagcggaaacatccagtacatgcttctcggtaatgttggggaaactgatgatggattgatcagcttcgcattgggttgcgtaaacctgcgaaagcttgaactcaggagttgctgcttcagcgagcgagcactggcccttgcaatactacatatgccttccctgaggtacgtatgggttcagggctacaaagcgtctcaaaccggccgagacctcatgctcatggcaaggcccttctggaacatagagtttacacctcccaatcctaagaacggaggttggctgatggaagatggggagccttgtgtagatagtcacgctcagatacttgcataccactccctcgccggtaagaggctggactgcccacaatccgtggttcctttgtatcctgcgtgagtgtaaatagactaagctggtgtctttccttagcctcctggtcaacaagaatggtgttgataactcgatatatgcggttattgtatggatctagatggctagctgctacgtactgtaataagctactagtagctgagagatgtcctggaataagcccttgctatttttgcctaaaaaaaaaaaaaaaa

SEQ ID NO: 25 - protein, 265 aa, Arachis hypogaeal
RHCKKLQRLWIMDSIGDKGLGVVANTCKELQELRVFPSDNIGQHAAVTEKGLVAISMGCPKLHSLLYFCHQMTNAALITVAKNCPNFIRFRLAILDATKPDPDTNQPLDEGFGAIVQSCRRLRRLSLSGQLTDKVFLYIGMYAEQLEMLSIAFAGESDKGMLYVLNGCKKLRKLEIRDCPFGNTALLTDVGKYETMRSLWMSSCEVTVGACKVLAMKMPRLNVEIFNENEPADCEPDDVQKVEKMYLYRTLAGKRKDAPEYVWTL

SEQ ID NO: 26 - DNA, 900 bp, Arachis hypogaeal
cgtcactgcaagaaacttcagcgcttatggataatggattccattggagataaagggctaggtgttgtagctaacacatgtaaggaattgcaagaattgagggtttttccttccgacaacattggtcagcatgcggctgtcacagagaagggattggttgcgatatctatgggctgcccgaaacttcactcattgctctacttctgccaccagatgacaaatgctgccctaataactgtggccaagaactgcccgaattttatccgctttaggttggccatccttgacgcaacaaaacccgaccccgacacaaatcagccactggatgaaggttttggggcgattgtgcaatcttgcaggcgtcttaggcggctttccctctctggccagctgactgataaggtattcctctacatcggaatgtatgctgagcagcttgagatgttgtccattgcctttgccggggagagcgacaaggggatgctctatgttctgaacggatgcaagaagctccgcaagcttgagatcagggactgccctttcggcaacacggcacttctgacagacgtagggaagtatgaaacaatgcgatccctttggatgtcgtcgtgcgaagtaaccgtcggagcatgcaaggtgctagcaatgaagatgccgaggctaaatgttgagatcttcaacgagaatgagccagccgactgcgagccggatgatgtgcagaaggtggagaagatgtacttgtaccggacattggctgggaagaggaaagatgcaccggaatatgtatggaccctttaggtgcatttttaggtcaattttaattttattgttattattgagcagtttgtacgttaggctgacttattaatgcaattttagccttgtgtagtggttggtttg

SEQ ID NO: 27 - protein, 173 aa, Triticum aestivum
MATSPVLFLLLAVFAAGASAATFNIKNNCGFTIWPAGIPVGGGFALGSGQTSSINVPAGTQAGRIWARTGCSFNGGSGSCQTGDCGGQLSCSLSGRPPATLAEYTIGGGSTQDFYDISVIDGFNLAMDFSCSTGDALQCRDPSCPPPQAYQHPNDVATHACSGNNNYQITFCP

SEQ ID NO: 28 - DNA, 674 bp, Triticum aestivum
cctacagcaaagctcgagcatagcaacagcactaaagctaactagagcttccagcaatggcgacctccccggtgctcttcctcctcctcgctgttttcgccgccggtgccagcgcggccaccttcaacatcaagaacaactgtggcttcacaatttggccggcgggcatcccggtgggtgggggcttcgcgctgggctcagggcagacgtccagcatcaacgtgcccgcgggcacccaagccgggaggatatgggcccgcaccgggtgctccttcaatggcggtagcgggagctgccagaccggcgactgcggcggccagctatcctgctccctctccgggcggccaccagcaacgctggccgagtacaccatcggcggcggcagcacccaggacttctacgacatctcggtgatcgacggcttcaaccttgccatggacttctcgtgcagcaccggcgacgcgctccagtgcagggaccccagctgcccgccgccgcaagcctaccaacaccccaacgacgtcgccacacacgcctgcagtggcaataataactaccagatcaccttctgtccatgaagcctctatacgtcgcaccgcgaatcaataaaaggcgtacgtagatatacggccatataaataaaaggtgtactgcttaataaaaaaaaaaaaaaa

SEQ ID NO: 29 - protein, 173 aa, Triticum aestivum
MATSAVLFLLLAVFAAGASAATFNIKNNCGSTIWPAGIPVGGGFELGAGQTSSINVPAGTKAGRIWARTGCSFNGGSGSCRTGDCGGQLSCSLSGRPPATLAEYTIGGGGTQDFYDISVIDGFNLAMDFSCSTGDALQCRDPSCPPPQAYQHPNDQATHACSGNNNYQITFCP

SEQ ID NO: 30 - DNA, 663 bp, Triticum aestivum
ccacgcgtccgatggcgacctccgcggtgctcttcctcctcctcgctgtttttgccgccggtgccagcgcggccaccttcaacatcaagaacaactgcggctccacaatttggccggcgggcatcccggtgggtgggggcttcgagctgggcgcaggccagacgtccagcatcaacgtgcccgcgggcaccaaagccgggaggatatgggctcgcaccgggtgctccttcaatggcggcagcgggagctgccggaccggcgactgcggcggccagctgtcctgctccctctccgggcggccaccagcaacgctggccgagtacaccatcggcggcggcggcacccaggacttctacgacatctcggtgatcgacggcttcaaccttgccatggacttctcgtgcagcaccggcgacgcgctccagtgcagggatcccagctgcccgccgccgcaagcctaccaacaccccaacgaccaagccacacacgcctgcagtggcaataataactaccagatcaccttctgcccatgaaggctgttcacgtcgcaccacgaatcaataaaaggcgtacgtagatatacggccctataaataaaaggcgtgctgcttaaaaaaaaaaaaaaaaaaaaaaaaaaaaaaaaaaaaaaaaaaaaaaaaaaaa

SEQ ID NO: 31 - protein, 173 aa, Hordeum vulgare
MSTSAVLFLLLAVFAAGASAATFNIKNNCGSTIWPAGIPVGGGFELGSGQTSSINVPAGTQAGRIWARTGCSFNGGSGSCQTGDCGGQLSCSLSGRPPATLAEFTIGGGSTQDFYDISVIDGFNLAMDFSCSTGDALQCRDPSCPPPQAYQHPNDVATHACSGNNNYQITFCP

SEQ ID NO: 32 - protein, 173 aa, Triticum urartu
MATSAVLFLLLAVFAAGASAATFNIKNNCGSTIWPAGIPVGGGFALGAGQTSSINVPAGTKAGRIWARTGCSFNGGSGSCRTGDCGGQLSCSLSGRPPATLAEYTIGGGSTQDFYDISVIDGFNLAMDFSCSTGDALQCRDPSCPPPQAYQHPNDQATHACSGNNNYQITFCP

SEQ ID NO: 33 - protein, 173 aa, Aegilops tauschii
MATSPVLFLLLAVFAAGASAATFNIKNNCGSTIWPAGIPVGGGFALGAGQTSSINVPAGTKAGRIWARTGCSFNGGSGSCQTGDCGGQLSCSLSGRPPATLAEYTIGGGSTQDFYDISVIDGFNLAMDFSCSTGDALQCRDPSCPPPQAYQHPDDRATHACNGNSNYQITFCP

SEQ ID NO: 34 - protein, 173 aa, Secale cereale
MATSAVLFLLFAVFAAGASAATFNIKNNCGSTIWPAGIPVGGAFALGSGQTSSINVPAGTQAGRIWARTGCSFNGGTGSCQTGDCGGQLSCSLSGRPPATLAEFTIGGGSTQDFYDISVIDGFNLAMDFSCSTGDALQCRDPSCPPPQAYQHPNDMATHACRGNSNYQITFCP

SEQ ID NO: 35 - DNA, 1151 bp, Secale cereale
ggcacgaggctaactagagcttgcagcaatggcgacctctgcggtgctcttcctcctcttcgctgtttttgccgccggtgccagcgcggccaccttcaacatcaagaacaactgcggctccacaatttggccggcgggcatcccggtgggtggggcgttcgcgctgggctcaggccagacgtccagcatcaacgtacccgcaggaacccaagccgggaggatatgggcccgcaccgggtgctccttcaatggcggtacggggagctgccagaccggcgactgcggtggccagctgtcctgctccctctccgggcggccaccagcaacgcttgccgagttcaccatcggcggcggcagcacccaggacttctacgacatctcggtgatcgacggcttcaaccttgccatggacttctcatgcagcaccggcgacgcgctacagtgcagggatcccagctgcccaccgccgcaagcctaccaacaccccaacgacatggccacacacgcctgcagaggcaatagtaactaccagatcaccttctgcccatgaagcatgtttacgtcgcacctcccatctataaaggcgtacgtagatatatggccgtataaattaaaggtgtgctgcttaatactccctctgtaaactaatataaaagcatttagatcactaaagtagtgttctaaacactcttatattagtttacggagggagtacatcacagatcacactgtcatattacagccactgtactactatattgaatggcagaggagccgattccgggcagagaagccgaagcaggggagccagagagagagagagagttgaaggaagagaggatatattttcgctcactctactcactactgtgagggttttattgatactagtaagcttgtacgtgcaaatgcacgtctcgactaatattacaaacgaaggtctacgcgtcggccggcggatctttttaaagatccgccgcctcacgagccgtccgatataagttgcttaacatgctatcccacttcgcaacaatagtgttgtttcagaaacaatggtgactattccaacaaagagctttatttcagaaaaaaaaaaaaaaaaaaaaaaaaaaaaaaaaaaaaaaaaaaaaaaaaaaaaaaaaaaaaaaaaa

SEQ ID NO: 36 - protein, 169 aa, Avena sativa
MATSSAVLFLLLAVFAAGASAATFRITNNCGFTVWPAGIPVGGGFQLNSKQSSNINVPAGTSAGRIWGRTGCSFNNGRGSCATGDCAGALSCTLSGQPATLAEYTIGGSQDFYDISVIDGFNLAMDFSCSTGVALKCRDANCPDAYHHPNDVATHACNGNSNYQITFCP

SEQ ID NO: 37 - protein, 171 aa, Triticum aestivum
MATSAVLFLLLAVFAAGASAATFYIKNNCGSTIWPAGIPVGGGFALGSGQTASINVPAGTKAGRIWARTGCSFNGGSGSCQTGDCGGQLSCSLSGRPPATLTENTIGGAQDFYDISVIDGFNLAMDFSCGTGDALQCRDPSCPPPQAYQHPNDVATHACNGNSNYQITFCP

SEQ ID NO: 38 - DNA, 516 bp, Triticum aestivum
atggcgacctccgcggtgctcttcctcctcctggctgttttcgccgccggtgccagcgcggccaccttctacatcaagaacaactgcggctccacaatttggccggcgggcatcccggtgggtgggggcttcgcgctgggctcaggccagacggccagcatcaacgtgcccgcgggcaccaaagccgggaggatatgggcccgcaccgggtgctccttcaatggcggtagcgggagctgccagaccggcgactgcggaggccagctgtcctgctccctctccgggcggccaccggcaacgctgaccgagaacaccatcggcggcgcccaagacttctacgacatctcggtgatcgacggcttcaaccttgccatggacttctcgtgcggcaccggcgacgcgctccagtgcagggaccccagctgcccgccgccgcaagcctaccaacaccccaacgacgtcgccacacacgcctgcaatggcaacagtaactaccagatcaccttctgtccatga

SEQ ID NO: 39 - protein, 171 aa, Zea mays
MTSSSVFFLLLACFATCAGAATFTVRNNCGFTVWPAGIPVGGGTQLNPGSTWTVNVQAGTSGGRIWGRTGCSFSGGRGRCATGDCGGAYSCSLSGQPPATLAEFTIGGGSNHDYYDISVIDGYNLPMDFSCSTGAALRCRDSGCPDAYHQPNDPKTRSCNGNSNYQVVFCP

SEQ ID NO: 40 - DNA, 756 bp, Zea mays
actatatacgtatatagacgtggtcaggatgacgtcgtcctccgtcttcttcctcctgctcgcttgcttcgccacatgcgccggcgccgccacgttcacggtacgaaacaactgcgggttcacggtgtggccagcaggcatcccggtcggcggcggcacgcagctgaacccgggctcgacgtggactgtcaacgtgcaggccgggaccagcggaggcaggatctggggccgcaccggctgctccttcagcggcggccggggccgctgcgcgacgggcgactgcggaggcgcctactcctgcagcctgtccgggcagcccccggcgacgctggccgagttcaccatcggcggcggcagcaaccacgactactacgacatctcggtgatcgacgggtacaacctgcccatggacttctcgtgcagcaccggcgccgccctccggtgcagggactccggctgccccgacgcgtaccaccagcccaacgaccctaagacccgttcgtgcaacggcaatagcaactaccaggtcgtcttctgcccttgatgtacatgcatgcagcttgccatgagcccatgcatgcatgagagcgtagcggcatatatacactaattgtccgtcaactcacatgcacgtacctactagtgaagttgataaataagacgatcgatatctgactcgtccaatgcagcatgtgtgtacggtgtgatccttatactttcctacatatatggaactttttaaagtgagtaaagtgc

SEQ ID NO: 41 - protein, 173 aa, Sorghum bicolor
MAASSSSILLLFLAATLMASGTHAATFTIKNNCGFTVWPAATPVGGGTQLNSGGTWTINVPAGTSSGRVWGRTGCSFNGNSGSCQTGDCGGALACTLSGKPPLTLAEFTIGGSQDFYDISVIDGFNIGMAFSCSTGVGLVCRDSSCPDAYHNPPDRKTHACGGNSNYQVTFCP

SEQ ID NO: 42 - DNA, 1234 bp, Sorghum bicolor
aagcagctcgcctatttccacaagaaagaagctcgcatagtattagcatcaatatatggccgcctcatcgtcctcgatcctcctgcttttcctcgccgccaccttgatggccagcggcactcacgcggcgaccttcaccatcaagaacaactgcgggttcacggtgtggccggcggcgaccccagtcggcgggggcacgcagctgaactcaggcgggacgtggacgatcaacgtgccggccggcaccagctccggccgcgtctggggccgcacgggctgctccttcaacggcaacagcgggagctgccagacgggcgactgcggcggcgcgctcgcctgcaccctctccggcaagcctccgctgacgctggccgagttcacgatcggcggcagccaggacttctacgacatctcggtcatcgacggcttcaacatcggcatggccttctcctgcagcaccggcgtcgggctggtgtgcagggactcgagctgccctgacgcataccacaatcctcccgacaggaagacccatgcctgcggcggcaacagcaactaccaggtcaccttctgcccgtgatgatgaggcagcaagtatatatatgcatggcttctgtatcgcatgcatgtatttacgtatacggcacctagcaggggatgtcggataggagagtgaataagacgtgtcgtggtagcgtacatgtccaagcgtgcatgcatgcatgtgcacgcgggcatatgtacctggaactgtgtgtacttacagtatactccagcagtataataatatgataaaatataataatagtaagacactgtcatgtcatgctagaaagcagggattaaaaaaagtgaaggtatattatacggctacactagaaagcgaccttttgggtcgatggtgacagattcagccactagacacacgtgtgtttgatcgagtcaaacaaaagttatattaggcaggacgaattaaaaaaatatttagagaagattctactactttactactttaagataattttaaccccttcttaagtggagcgccaaccttaaagagaaagatcagaagtagatcatcaagatgaaggggctgccgtcacaagttggctcaaagacttcaagactagctcggttaatccttctaatgtagtggaaatctagtcgtgtagttgtatttggttttcgctgtactgctatgttttgaactggtaaccccagtcgtgatgctctaatg

SEQ ID NO: 43 - protein, 176 aa, Oryza sativa
MASPATSSAILVVVLVATLAAGGANAATFTITNRCSFTVWPAATPVGGGRQLSPGDTWTINVPAGTSSGRVWGRTGCSFDGSGRGSCSTGDCGGALSCTLSGQPPLTLAEFTIGGSQDFYDLSVIDGFNVGMSFSCSSGVTLTCRDSSCPDAYHSPNDRKTHACGGNSNYQVVFCP

SEQ ID NO: 44 - protein, 173 aa, Setaria italica
MAASSVVVFLLLAAFVAGASAATFTIKNNCPYTVWPAATPVGGGRQLNSGQTWTLDVPAGTSSGRIWGRTGCSFSNGRGRCASGDCGGALSCTLSGQPPLTLAEFTIGSGDKQDFYDISVIDGYNLPMDFSCSNGRNLQCRAPRCPDAYLFPSDNSKNHPCRGNSNYRVTFCP

SEQ ID NO: 45 - DNA, 731 bp, Setaria italica
tcacttgtagcagaccaaccagtagttgtccagtaccaatggcggcctcctctgtcgtcgtcttcctcctcctcgcggccttcgtcgccggcgccagcgcggccaccttcaccatcaagaacaactgcccctacacggtgtggccggcggcgacccccgtcggcggcggcaggcagctcaactcaggccagacgtggaccctcgacgtgcccgctggcaccagttccggcaggatctggggccgcaccggctgctccttcagcaacggccgcggccggtgcgcttcgggcgactgcggcggcgcgctctcctgcacgctctccgggcagccgccgctgactctggccgagttcaccatcgggagcggcgacaagcaggacttctacgacatctcggtgatcgacgggtacaacctgcccatggacttctcctgcagcaatggcaggaacctgcagtgccgtgcccctcgctgccccgacgcgtacctgttccccagcgacaattccaagaaccacccgtgccgtggcaacagcaactacagggtcaccttctgcccatgaacggtggtcgaacgatggtgcagtaatgcatcatcatggccacgtacgggaaagaaataataagttaatgaatgaataagacgacctttgggtcgtgcatgcgtgcatgcatctgatctatactatgtactttcatggaacttcagttaataatttgcacgtcttctgcta

SEQ ID NO: 46 - DNA, 23 bp, Artificial Sequence
  A synthetic primer sequence
ccgtttgtagagccataattgca

SEQ ID NO: 47 - DNA, 25 bp, Artificial Sequence
  A synthetic primer sequence
aggttgcctgagtcacagttaagtg

SEQ ID NO: 48 - DNA, 19 bp, Artificial Sequence
  A synthetic primer sequence
cgctgaccgagaacaccat

SEQ ID NO: 49 - DNA, 22 bp, Artificial Sequence
  A synthetic primer sequence
tcgatcaccgagatgtcgtaga

SEQ ID NO: 50 - DNA, 20 bp, Artificial Sequence
  A synthetic primer sequence
tggcgtactactccctttct

SEQ ID NO: 51 - DNA, 22 bp, Artificial Sequence
  A synthetic primer sequence
gagacaccataccggcttaatg